(19) United States
(12) Patent Application Publication
(10) Pub. No.: US 2023/0212690 A1
(43) Pub. Date: Jul. 6, 2023

(54) MULTIMODAL ANALYSIS OF CIRCULATING TUMOR NUCLEIC ACID MOLECULES

(71) Applicant: UNIVERSITY HEALTH NETWORK, Toronto (CA)

(72) Inventors: Scott Victor BRATMAN, Toronto (CA); Justin Matthew BURGENER, Toronto (CA); Daniel DINIZ DE CARVALHO, Toronto (CA)

(21) Appl. No.: 18/067,661

(22) Filed: Dec. 16, 2022

Related U.S. Application Data
(63) Continuation of application No. PCT/CA2021/050842, filed on Jun. 18, 2021.
(60) Provisional application No. 63/041,151, filed on Jun. 19, 2020.

(51) Int. Cl. C12Q 1/6886; G16B 30/00

(52) U.S. Cl. CPC C12Q 1/6886; G16B 30/00; C12Q 2600/154; C12Q 2600/156

(57) ABSTRACT

In an aspect, there is provided a method of detecting the presence of ctDNA from cancer cells in a subject comprising: (a) providing a sample of cell-free DNA from a subject; (b) subjecting the sample to library preparation to permit subsequent sequencing of the cell-free methylated DNA; (c) optionally adding a first amount of filler DNA to the sample, wherein at least a portion of the filler DNA is methylated, then further optionally denaturing the sample; (d) capturing cell-free methylated DNA using a binder selective for methylated polynucleotides; (e) sequencing the captured cell-free methylated DNA; (f) comparing the sequences of the captured cell-free methylated DNA to control cell-free methylated DNAs sequences from healthy and cancerous individuals; (g) identifying the presence of DNA from cancer cells if there is a statistically significant similarity between one or more sequences of the captured cell-free methylated DNA and cell-free methylated DNAs sequences from cancerous individuals; wherein in at least one of the capturing step, the comparing step or the identifying step, the subject cell-free methylated DNA is limited to a sub-population according to a fragment length metric.

### CROSS REFERENCES

This application is a continuation application of International Application No. PCT/CA2021/050842, filed Jun. 8, 2021, which claims the benefit of U.S. provisional patent application No. 63/041,151, filed Jun. 19, 2020, which are each entirely incorporated herein by reference.

### BACKGROUND

Circulating tumor DNA (ctDNA) has increasingly demonstrated potential as a non-invasive, tumor-specific biomarker for routine clinical use. ctDNA is derived from tumor cells predominately undergoing cell-death and released into circulation of various bodily fluids including blood. In most cancer patients, the majority of blood-derived cell-free DNA originates from peripheral blood leukocytes (PBLs); therefore, identification of tumor-derived genetic and epigenetic alterations are required for ctDNA detection and quantification. In addition, the fraction of ctDNA observed may range from <0.1% to 90% of total cell-free DNA at diagnosis depending on several factors including primary site of the tumor and disease burden. ctDNAs has been providing non-invasive access to the tumor's molecular landscape and disease burden. Methods for detecting ctDNA with increased sensitivity especially in subjects with lower abundance of ctDNA are needed.

### INCORPORATION BY REFERENCE

All publications, patents, and patent applications mentioned in this specification are herein incorporated by reference to the same extent as if each individual publica-tion, patent, or patent application was specifically and indi-vidually indicated to be incorporated by reference.

### SUMMARY

In an aspect, there is provided a method of detecting the presence of ctDNA from cancer cells in a subject comprising:
(a) providing a sample of cell-free DNA from a subject;
(b) subjecting the sample to library preparation to permit subsequent sequencing of the cell-free methylated DNA;
(c) optionally adding a first amount of filler DNA to the sample, wherein at least a portion of the filler DNA is methylated, then further optionally denaturing the sample;
(d) capturing cell-free methylated DNA using a binder selective for methylated polynucleotides;
(e) sequencing the captured cell-free methylated DNA;
(f) comparing the sequences of the captured cell-free methylated DNA to control cell-free methylated DNAs sequences from healthy and cancerous individuals;
(g) identifying the presence of DNA from cancer cells if there is a statistically significant similarity between one or more sequences of the captured cell-free methylated DNA and cell-free methylated DNAs sequences from cancerous individuals;
wherein in at least one of the capturing step, the comparing step or the identifying step, the subject cell-free methylated DNA is limited to a sub-population according to a fragment length metric.

In as aspect, the present disclosure provides methods for determining whether a subject has or is at risk of having a disease. The methods comprise: subjecting a plurality of nucleic acid molecules derived from a cell-free nucleic acid sample obtained from said subject to sequencing to generate at least one profile selected from the group consisting of (i) a methylation profile, (ii) a mutation profile, and (iii) a fragment length profile; and processing said at least one profile to determine whether said subject has or is at risk of said disease at a sensitivity of at least 80% or at a specificity of at least about 90%, wherein said cell-free nucleic acid sample comprises less than 30 nanograms (ng)/milliliter (ml) of said plurality of nucleic acid molecules.

In some embodiments, the cell-free nucleic acid sample comprises less than 10 ng/ml of said plurality of nucleic acid molecules. In some embodiments, the cell-free nucleic acid sample comprises less than 5 ng/ml of said plurality of nucleic acid molecules. In some embodiments, the cell-free nucleic acid sample comprises less than 1 ng/ml of said plurality of nucleic acid molecules. In some embodiments, the subjecting of (a) generates at least two profiles selected from the group consisting of (i), (ii) and (iii). In some embodiments, the at least two profiles comprise said methylation profile and said fragment length profile.

In some embodiments, the at least two profiles comprise said mutation profile and said fragment length profile. In some embodiments, the at least two profiles comprise said methylation profile and said mutation profile. In some embodiments, the subjecting of (a) generates said methylation profile, said mutation profile, and said fragment length profile.

In another aspect, the present disclosure provides methods for processing a cell-free nucleic acid sample of a subject to determine whether said subject has or is at risk of having a disease. The methods comprise providing said cell-free nucleic acid sample comprising a plurality of nucleic acid molecules; subjecting said plurality of nucleic acid molecules or derivatives thereof to sequencing to generate a plurality of sequencing reads; computer processing said plurality of sequencing reads to identify, for said plurality of nucleic acid molecules, (i) a methylation profile, (ii) a mutation profile, and (iii) a fragment length profile; and using at least said methylation profile, said mutation profile and said fragment length profile to determine whether said subject has or is at risk of having said disease.

In some embodiments, the disease comprises a cancer. In some embodiments, the cancer is selected from the group consisting of the cancer is selected from the group consisting of adrenal cancer, anal cancer, bile duct cancer, bladder cancer, bone cancer, brain/cns tumors, breast cancer, castleman disease, cervical cancer, colon/rectum cancer, endometrial cancer, esophagus cancer, ewing family of tumors, eye cancer, gallbladder cancer, gastrointestinal carcinoid tumors, gastrointestinal stromal tumor (gist), gestational trophoblastic disease, hodgkin disease, kaposi sarcoma, kidney cancer, laryngeal and hypopharyngeal cancer, leukemia (acute lymphocytic, acute myeloid, chronic lymphocytic, chronic myeloid, chronic myelomonocytic), liver cancer, lung cancer (non-small cell, small cell, lung carcinoid tumor), lymphoma, lymphoma of the skin, malignant mesothelioma, multiple myeloma, myelodysplastic syndrome, nasal cavity and paranasal sinus cancer, nasopharyngeal cancer, neuroblastoma, non-hodgkin lymphoma, oral cavity and oropharyngeal cancer, osteosarcoma, ovarian cancer, penile cancer, pituitary tumors, prostate cancer, retinoblastoma, rhabdomyosarcoma, salivary gland cancer, sarcoma—adult soft tissue cancer, skin cancer (basal and squamous cell, melanoma, merkel cell), small intestine cancer, stomach cancer, testicular cancer, thymus cancer, thyroid cancer, uterine sarcoma, vaginal cancer, vulvar cancer, waldenstrom macroglobulinemia, wilms tumor, squamous cell carcinoma, and head and neck squamous cell carcinoma. In some embodiments, the cancer is squamous cell carcinoma. In some embodiments, the cancer is head and neck squamous cell carcinoma.

In some embodiments, the plurality of cell-free nucleic acid molecules comprises circulating tumor nucleic acid molecules. In some embodiments, the circulating tumor nucleic acid comprises circulating tumor DNA. In some embodiments, the circulating tumor nucleic acid comprises circulating tumor RNA. In some embodiments, the methylation profile comprises a plurality of Differentially Methylated Regions (DMRs). In some embodiments, the plurality of DMRs is ctDNA derived. In some embodiments, a plurality of DMRs derived from peripheral blood leukocytes is removed from said methylation profile. In some embodiments, the plurality of DMRs comprises at least about 56 genomic regions with hypo-methylation levels compared to corresponding genomic regions from a normal healthy subject. In some embodiments, the plurality of DMRs comprises at least about 941 genomic regions with hyper-methylation levels compared to corresponding genomic regions from a normal healthy subject. In some embodiments, a DMR comprises a size of at least about 300 bp. In some embodiments, a DMR comprises a size of at least about 100 bp to at least about 200 bp. In some embodiments, a DMR comprises a size of at least about 100 bp to at least about 150 bp. In some embodiments, a DMR comprises at least 8 CpG genomic islands. In some embodiments, the normal healthy subject comprises a same set of risk factors as said subject.

In some embodiments, the mutation profile comprises a missense variant, a nonsense variant, a deletion variant, an insertion variant, a duplication variant, an inversion variant, a frameshift variant, or a repeat expansion variant. In some embodiments, any variant that is present in a genomic DNA sample obtained from a plurality of peripheral blood leukocytes, wherein said plurality of peripheral blood leukocytes is obtained from said subject, is removed from the mutation profile. In some embodiments, any variant that is derived from clonal hematopoiesis is removed from said mutation profile. In some embodiments, the mutation profile does not comprise a variant of gene DNMT3A, TET2, or ASXL1. In some embodiments, the mutation profile does not comprise a canonical cancer driver gene. In some embodiments, the mutation profile comprises non-canonical cancer driver gene, where said non-canonical gene is GRIN3A or MYC.

In some embodiments, the fragment length profile comprises selecting cell free nucleic acid molecules based on a range of fragment length of about at least 80 bp to 170 bp. In some embodiments, the fragment length profile comprises selecting cell free nucleic acid molecules based on a range of fragment length of about at least 100 bp to 150 bp. In some embodiments, the circulating tumor nucleic acid molecules are enriched.

In some embodiments, the methods further comprise mixing said cell free nucleic acid sample with a filler DNA molecules to yield a DNA mixture. In some embodiments, the filler DNA molecules comprise a length of about 50 bp to 800 bp. In some embodiments, the filler DNA molecules comprise a length of about 100 bp to 600 bp. In some embodiments, the filler DNA molecules comprises at least about 5% methylated filler DNA molecules. In some embodiments, the filler DNA molecules comprises at least about 20% methylated filler DNA. In some embodiments, the filler DNA molecules comprises at least about 30% methylated filler DNA. In some embodiments, the filler DNA molecules comprises at least about 50% methylated filler DNA.

In some embodiments, the methods further comprise incubating said DNA mixture with a binder that is configured to bind methylated nucleotides to generate an enriched sample. In some embodiments, the binder comprises a protein comprising a methyl-CpG-binding domain. In some embodiments, the protein is a MBD2 protein. In some embodiments, the binder comprises an antibody. In some embodiments, the antibody is a 5-MeC antibody. In some embodiments, the antibody is a 5-hydroxymethyl cytosine antibody. In some embodiments, the sequencing does not comprise bisulfite sequencing. In some embodiments, the cell-free nucleic acid sample comprises a blood sample. In some embodiments, the blood sample comprises a plasma sample. In some embodiments, the methods further comprise detecting an origin of cancer tissue.

In some embodiments, the methods further comprise generating a report comprising a prognosis of said subject's survival rate. In some embodiments, the methods further comprise providing a treatment to said subject. In some embodiments, subsequent to treatment of said disease, the methods further comprise providing a second report indicating whether said treatment is effective.

In another aspect, the present disclosure provides methods for determining whether a subject has or is at risk of having a condition, comprising: assaying a cell-free nucleic acid molecule from at least a portion of a sample from said subject; detecting a methylation level of at least a portion of said cell-free nucleic acid molecule comprised in a differentially methylated region (DMR) listed in Table 5; and comparing, using at least one computer processor, said methylation level detected in (b) to a methylation level of corresponding portion(s) of said cell-free nucleic acid molecules comprised in said DMR listed in Table 5.

In some embodiments, the cell-free nucleic acid molecule comprises ctDNA. In some embodiments, the methods comprise performing the sequence analysis, and wherein said sequencing analysis comprises a cell-free methylated DNA immunoprecipitation (cfMeDIP) sequencing. In some embodiments, the detecting comprises measuring a methylation level of at least a portion of said nucleic acid molecule comprised in: six or more, ten or more, fifteen or more, twenty or more, thirty or more, forty or more, fifty or more, sixty or more, seventy or more, eighty or more, ninety or more, or one hundred or more DMRs listed in Table 5.

In another aspect, the present disclosure provides methods method for determining whether a subject has a higher survival rate after receiving a treatment for a disease, comprising: assaying a cell-free nucleic acid molecule from at least a portion of a sample from said subject; detecting a methylation level of at least a portion of said cell-free nucleic acid molecule comprised in a differentially methylated region (DMR) listed in Table 6; and processing, using at least one computer processor, said methylation level detected in (b) to a methylation level of corresponding portion(s) of said cell-free nucleic acid molecules comprised in said DMR listed in Table 6.

In some embodiments, the cell-free nucleic acid molecule comprises ctDNA. In some embodiments, the detecting comprises providing a composite methylation score (CMS). In some embodiments, the CMS comprises a sum of beta-values of DMRs listed in Table 6. In some embodiments, a higher CMS indicates an inferior survival for said subject. In some embodiments, the CMS is not dependent on an abundance of ctDNA. In some embodiments, the disease is squamous cell carcinoma. In some embodiments, the cancer is head and neck squamous cell carcinoma.

In another aspect, the present disclosure provides systems for determining whether a subject has or is at risk of having a disease, comprising one or more computer processors that are individually or collectively programmed to implement a process comprising: subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and processing said at least one profile to determine whether said subject has or is at risk of said disease at a sensitivity of at least 80% or at a specificity of at least about 90%, wherein said cell-free nucleic acid sample comprises less than 30 ng/ml of said plurality of nucleic acid molecules.

In another aspect, the present disclosure provides systems for processing a cell-free nucleic acid sample of a subject to determine whether said subject has or is at risk of having a disease, comprising one or more computer processors that are individually or collectively programmed to implement a process comprising: providing said cell-free nucleic acid sample comprising a plurality of nucleic acid molecules; subjecting said plurality of nucleic acid molecules or derivatives thereof to sequencing to generate a plurality of sequencing reads; computer processing said plurality of sequencing reads to identify, for said plurality of nucleic acid molecules, (i) a methylation profile, (ii) a mutation profile, and (iii) a fragment length profile; and using at least said methylation profile, said mutation profile and said fragment length profile to determine whether said subject has or is at risk of having said disease.

### BRIEF DESCRIPTION OF FIGURES

The patent or application file contains at least one drawing executed in color. Copies of this patent or patent application publication with color drawing(s) will be provided by the Office upon request and payment of the necessary fee. These and other features of the preferred embodiments of the invention will become more apparent in the following detailed description in which reference is made to the appended drawings wherein:

FIG. 1. Utilization of PBL-filtering for detection of ctDNA by CAPP-Seq. A) Mutant allele fraction of candidate SNVs identified in matched patient plasma and/or PBLs. Pearson's correlation was performed on SNVs strictly found in both matched patient plasma and PBLs. Candidate SNVs found only in patient plasma are denoted within the dashed red box. B) Oncoprint of candidate SNVs identified in both matched patient plasma and PBLs. The top histogram denotes the number of SNVs per patient whereas the right histogram denotes the number of patients with a specified gene mutated. C) Mean MAF of candidate SNVs across HNSCC patient cfDNA (red circle) and PBL (blue circle) before and after removal of PBL-associated SNVs. Patients with SNVs absent after PBL filtering are indictive of false positive detection of ctDNA. E) Oncoprint of selected PBL-filtered SNVs identified in 20/32 HNSCC patients. The top and right histograms denote that as previously described in (B). F) Mean mutant allele percentage of PBL-filtered SNVs across all HNSCC patients. For each SNV per patient, the mutant allele percentage was calculated by the fraction of reads containing the SNV of interest, compared to reads that contained the native sequence overlapping the SNV base-pair position

FIG. 2. Utilization of PBL-filtering for detection of ctDNA by CAPP-Seq. B) Mutant allele fraction of candidate SNVs identified in matched patient plasma and/or PBLs. Pearson's correlation was performed on SNVs strictly found in both matched patient plasma and PBLs. Candidate SNVs found only in patient plasma are denoted within the dashed red box. C) Oncoprint of candidate SNVs identified in both matched patient plasma and PBLs. The top histogram denotes the number of SNVs per patient whereas the right histogram denotes the number of patients with a specified gene mutated. D) Mean MAF of candidate SNVs across HNSCC patient cfDNA (red circle) and PBL (blue circle) before and after removal of PBL-associated SNVs. Patients with SNVs absent after PBL filtering are indictive of false positive detection of ctDNA. E) Oncoprint of selected PBL-filtered SNVs identified in 20/32 HNSCC patients. The top and right histograms denote that as previously described in (B). F) Mean mutant allele percentage of PBL-filtered SNVs across all HNSCC patients. For each SNV per patient, the mutant allele percentage was calculated by the fraction of reads containing the SNV of interest, compared to reads that contained the native sequence overlapping the SNV base-pair position.

FIG. 3. Identification of informative regions for detection of ctDNA by cfMeDIP-seq. B) Pearson's correlation of 300-bp non-overlapping windows with >=8 CpGs from patient and healthy donor cfDNA cfMeDIP-seq profiles (n=52) against FaDu genomic DNA (gDNA) [1×1×52 comparisons], unmatched PBL gDNA [1×51×52 comparisons], and matched PBL gDNA [1×1×52 comparisons] MeDIP-seq profiles. C) Performance of in-silico PBL-depletion in healthy donor (right) and HNSCC (left) PBL MeDIP-seq profiles. Absolute methylation scores were calculated from MeDIP-seq counts via MeDEStrand (Methods). 300-bp non-overlapping windows before PBL-depletion (blue) correspond with all windows from chromosome 1-22 with >=8 CpGs (n=702,488). 300-bp non-overlapping windows after PBL-depletion (red) include an additional filter where the median absolute methylation across healthy donor PBLs is <0.1 (n=99,997). D) Workflow of ctDNA detection by differential methylation analysis of HNSCC and healthy donor cfMeDIP-seq profiles. cfMeDIP-seq profiles from HNSCC patients with detectable SNVs by CAPP-Seq (i.e. CAPP-Seq positive, n=20) were compared to healthy donors (n=20) within PBL-depleted windows to identify HNSCC-associated cfDNA methylation. Hyper- and hypo-methylated regions are denoted as regions with higher or lower methylation in the HNSCC cohort compared to healthy donors at an FDR<10%. E) Permutation analysis of hyper-methylated regions annotated by CpG site (n=10,000 total permutations). Significant enrichment/depletion is denoted as observed z-scores with a p-value less than 0.05. F, Permutation analysis of hyper-methylated regions within tumor-specific methylated cytosines from TCGA (n=1000 permutations total). Significant enrichment/depletion is denoted as observed z-scores with a p-value less than 0.05.

FIG. 4. Concordance of ctDNA detection and abundance between CAPP-Seq and cfMeDIP-seq profiles. A) Median fragment length of detected SNVs across HNSCC patients by CAPP-seq. For each patient, the median fragment length of each SNV and matched reference allele was measured. The distribution of median fragment length for each mutation or matched reference allele is shown per patient. Extremes of boxes and centerlines define upper and lower quartiles and medians, respectively. In cases with a single SNV, the coloured line denotes the median length of fragments containing the SNV or matched reference allele, respectively. B) Fragment length distributions within HNSCC hyper-methylated regions by cfMeDIP-seq. Fragment lengths from healthy donors were pooled prior to analysis, where each subsequent box denotes an individual HNSCC cfMeDIP-seq profile. Extremes of boxes and centerlines define upper and lower quartiles and medians, respectively. Individual HNSCC samples are ordered based on increasing mean methylation (RPKM) within the hyper-methylated regions. Dashed blue line defines the median fragment length across all healthy donors. C) Ratio of enrichment for hyper-DMR regions by fragments between 100-150 bp compared to enrichment for hyper-DMR regions by fragments between 100-220 bp. Ratios were converted to percent increase/decrease for ease of interpretation. D) Ratio of enrichment for hyper-DMR regions by fragments between 100-150 bp compared to enrichment for hyper-DMR regions by fragments between 100-220 bp.+ symbols denote HNSCC patients with detectable ctDNA by CAPP-Seq (CAPP-Seq positive). E) Supervised hierarchal classification of cfMeDIP-seq profiles limited to 100-150 bp, by log-transformed RPKM values across HNSCC hyper-methylated regions. RPKM values for each cfMeDIP-seq profile was log 2-transformed prior to Euclidean transformation and clustered using Ward's method. Methylation clusters were defined at a threshold of k=4. F), Relationship of mean mutant allele frequency and mean RPKM from identified SNVs and hyper-methylated regions by CAPP-seq and cfMeDIP-seq (limited to 100-150 bp), respectively. Points denote individual samples from HNSCC or healthy donor plasma. Solid red line and shaded grey area denotes the fitted linear regression model and associated 95% confidence interval, respectively. G) AUROC analysis based on methylation values (limited to 100-150 bp) within HNSCC hyper-methylated regions, comparing HNSCC to healthy donor cfMeDIP-seq profiles. Detection of ctDNA was defined as instances where mean methylation was above the max value across healthy donors. H) Kaplan-Meier curve analysis for overall survival of patients within methylation cluster 1+2+3, compared to methylation cluster 4. I+J) Comparison of median fragment lengths from CAPP-Seq and cfMeDIP-seq profiles (I) and median fragment length from CAPP-Seq and 100-150:151-220 bp ratio from cfMeDIP-seq profiles (J). Points defined individual HNSCC samples within methylation cluster 1 and 2. Solid red line and shaded grey area denotes the fitted linear regression model and 95% confidence interval, respectively.

FIG. 5. Prognostic utility of specific methylated regions within ctDNA detected by cfMeDIP-seq. A) Relationship of mean mutant allele fraction and mean RPKM from identified mutations and hyper-methylated regions by CAPP-seq and cfMeDIP-seq (limited to 100-150 bp), respectively. Points denote individual samples from HNSCC or healthy control plasma. Solid red line: fitted linear regression model. Grey boundaries: 95% confidence interval. B) Kaplan-Meier analysis depicting overall survival of patients with detectable ctDNA both by CAPP-Seq and cfMeDIP-seq (mean methylation above healthy controls within hyper-DMRs) C) Identification of prognostic regions based on disease-specific survival by multivariate Cox Proportional Hazard regression analysis across HNSCC primary tumors provided by the TCGA (n=520). Regions were defined as 300-bp windows as previously described. HumanMethylation450K data was obtained from the TCGA and beta-values from probe IDs overlapping with each region were averaged. Candidate regions for prognostic analysis was selected based on elevated methylation across primary tumors (n=520) compared to solid adjacent normal tissue (n=50) (Wilcoxon's test, adjusted p value <0.05, log 2FC>1). G-H) Spearman's correlation from methylation of a particular 300-bp region (boxes) to the RNA expression of a particular transcript. Regions with an absolute R value >=0.3 (denoted by dashed grey lines) were labeled as significant associations. Methylated regions which were prognostic for disease-specific survival of HNSCC patients provided by the TCGA (n=520) are denoted with a red outline. Prognostic regions which were further associated with RNA expression are denoted as solid red. Example prognostic methylated regions associated with RNA expression; (G) OSR1, (H) LINC01391 are provided. E) Kaplan-Meier curve of overall survival for HNSCC-TCGA patients based on total methylation across five regions affecting expression of ZNF323/ZSCAN1, LINC01391, GATA-AS1, OSR1, and STK3/MST2 respectively. Patients were stratified based on either being below (Blw med. blue) or above (Abv med. red) the median total methylation of the five regions previously identified in (D) across all primary tumors. F) Kaplan-Meier curve of overall survival as described in (E) for HNSCC plasma cohort with detectable ctDNA by CAPP-Seq. To calculate total methylation across the five genes with prognostic association, RPKM values were scaled accordingly across all hyper-DMR regions previously identified prior to survival analysis.

FIG. 6. Clinical utility of ctDNA detection by cfMeDIP-seq for longitudinal monitoring. A) ctDNA kinetics typically observed across patients throughout treatment. Complete clearance was defined as a change from detected ctDNA at diagnosis to a decrease in ctDNA abundance below the threshold of detection (i.e. 0.2%) at first available mid-/post-treatment timepoint. Partial clearance was defined as a change from detected ctDNA at diagnosis to a decrease (>=90%) in ctDNA abundance above the threshold of detection at first available mid-/post-treatment timepoint. No clearance was defined as an increase in ctDNA abundance in mid-/post-treatment samples compared to at diagnosis. lastFU=sample collection at last follow-up, RT=radiotherapy. B) Changes in ctDNA abundance at diagnosis to first available mid-/post-treatment timepoint across HNSCC patients (n=30). Red lines denote patients that demonstrated kinetics of no-clearance, whereas grey lines denote patients with kinetics of clearance/partial-clearance. C, Kaplan-Meier curve of recurrence-free survival. Patients were stratified based on kinetics of clearance (i.e. no clearance vs. clearance/partial clearance).

FIG. 7. Comparison of cfMeDIP-seq analysis performed on all or ctDNA-enriched fragments. ctDNA-enriched fragments are defined as fragments ranging from 100-150 bp in length. A) Mutant allele frequency of mutations identified by CAPP-Seq vs. mean RPKM values of previously identified HNSCC hyperDMRs in cfMeDIP-seq profiles containing all fragments (left) or ctDNA-enriched fragments (right). B) Area under the curve analysis (AUROC) for ctDNA detection in HNSCC cfMeDIP-seq profiles (CAPP-Seq positive only: red, CAPP-Seq positive and negative: blue) versus healthy donors. Results of cross-validation analysis using CAPP-Seq positive patients is also shown (replicates=50). Analysis is shown for cfMeDIP-seq profiles with all fragments (left) or ctDNA-enriched fragments (right). C) Kaplan-Meier analysis for recurrence-free survival based on longitudinal cfMeDIP-seq profiling with all fragments (left) or ctDNA-enriched fragments. Patients were classified as being positive for post-treatment ctDNA if they demonstrated methylation abundance within the previously identified hyperDMRs greater than 0.2% ctDNA.

FIG. 8. shows a computer system that is programmed or otherwise configured to implement methods provided herein FIG. 9. Sample characteristics of isolated cell-free DNA from HNSCC and healthy donors. A) Schematic defining timepoints of blood isolation. B) cfDNA yields (normalized to per mL of plasma) across timepoints for HNSCC patients as well as healthy donors (i.e. “Normal”).

FIG. 10. Analysis of the number of SNVs per HNSCC patient covered by the CAPP-Seq selector assessed either among all 364 patients in the HNSC TCGA cohort (blue diamonds) or using leave-one-out cross-validation (LOOCV; red squares).

FIG. 11. Oncoprint of all PBL-filtered SNVs identified in 20/32 HNSCC patients (Related to FIG. 2E).

FIG. 12. Related figures for identification of informative regions (related to FIGS. 3B and C). A) Median RPKM values of genome-wide (chromosomes 1-22) 300-bp non-overlapping bins based on >=n CpGs. B) Differential methylation analysis between HNSCC and healthy donor PBLs within PBL-depleted windows as described in FIG. 2B and Methods. Hypomethylated regions (i.e. regions with elevated methylation in healthy donor PBLs) are denoted in blue.

FIG. 13. Related figures to results of differential methylation analysis between HNSCC and healthy donor cfDNA samples within PBL-depleted windows (FIG. 2D). A) DMRs were defined based on the original 300-bp non-overlapping windows used for the initial analysis. DMRs immediately adjacent to each other were binned into their respective widths (i.e. two 300-bp windows are each independently defined as having a length of 600-bp). B) Permutation analysis of CpG features as defined in FIG. 2E, based on hypo-methylated regions.

FIG. 14. Supervised hierarchical clustering of TCGA primary tumors based on identified of cancer-specific differentially methylated cytosines. Cancer_type (column) refers to the classification of each primary tumor or PBL sample, whereas cancer_DMCs (row) refers to cancer-specific differentially methylated cytosines identified for each cancer type (PBLs excluded).

FIG. 15. Related figures to FIG. 4. A) Median fragment length of identified SNVS by CAPP-Seq per patient compared to mean mutant allele fraction. B) Median fragment length within hyper-DMRS by cfMeDIP-seq per patient compared to mean RPKM of hyper-DMRs.

FIG. 16. Related figures to CAPP-Seq and cfMeDIP-seq concordance analysis (FIG. 4E). A) Area under the curve values obtained from cross-validation analysis (n=50) of differentially methylated region calling between CAPP-Seq positive HNSCC cfDNA samples and healthy donors. B) Kaplan-Meir analysis for overall survival of HNSCC patients based on the detection of ctDNA by CAPP-Seq. C) and D) mean RPKM and mean mutant allele fraction of HNSCC patient samples stratified based on methylation cluster (FIG. 4D).

FIG. 17. Identification of regions of potential clinical utility (related to FIG. 6). A) Genome-track of genes currently used in commercially available liquid biopsy tests with overlap to HNSCC primary tumors within the TCGA as well as plasma-derived hyper-DMRs from our HNSCC cohort. Bottom dark blue bar with arrows denotes the direction of transcription for the specified gene. Red bars indicate location of 300-bp windows overlapping with hyper-DMRs from plasma of our HNSCC cohort as well as primary tumors from the TCGA. B-D) Spearman's correlation from methylation of a particular 300-bp region (boxes) to the RNA expression of a particular transcript. Regions with an absolute R value >=0.3 (denoted by dashed grey lines) were labeled as significant associations. Methylated regions which were prognostic for disease-specific survival of HNSCC patients provided by the TCGA (n=520) are denoted with a red outline. Prognostic regions which were further associated with RNA expression are denoted as solid red. Figures were generated for all five genes contained prognostic methylated regions associated with RNA expression; (B) GATA2-AS1, (C) ZNF323, (D), STK3.

FIG. 18. Extension of FIG. 6A, displaying changes in ctDNA abundance by cfMeDIP-seq throughout treatment for all HNSCC patients (n=32)

### DETAILED DESCRIPTION

In the following description, numerous specific details are set forth to provide a thorough understanding of the invention. However, it is understood that the invention may be practiced without these specific details.

The present disclosure provides methods, systems, and kits for multimodal analysis of ctDNA in determining a likelihood of a subject having cancer with high sensitivity and/or high specificity. Further, the present disclosure provides methods, systems, and kits for detecting minimal residual disease (MRD) after a cancer treatment, and for evaluating whether such cancer treatment is therapeutically effective.

Identification of specific molecular features from ctDNA prior to treatment may inform prognosis and/or be predictive response to therapy, whereas detection of ctDNA after treatment may aid in identification of MRD and aid in identifying patients at high risk of recurrence and/or death. To achieve robust sensitivity, most clinical studies utilize ctDNA detection methods interrogating few regions, matched tumor profiling, and/or cases of high ctDNA abundance. However, for cancers that harbor low levels of ctDNA or lack common/known aberrations across patients, additional strategies may be utilized to achieve similar degrees of sensitivity. Genome-wide profiling techniques may help improve sensitivity by covering considerably more regions; however, the amount of cell-free DNA and sequencing depth required to achieve detection below a fraction of 1% has been cost-prohibitive.

Two tailored genome-wide profiling techniques capable of highly sensitive ctDNA detection have been described. The first, CAncer Personalized Profiling by deep Sequencing (CAPP-Seq), utilizes a broad panel of hybrid-capture probes targeting over 100 genes to identify low allele frequency mutations. The second, cell-free Methylated DNA ImmunoPrecipitation sequencing (cfMeDIP-seq), enriches for methylated cfDNA fragments through use of an anti-5-methylcytosine (anti-5mC) antibody. The identification of mutations or hypermethylation events by these respective methods have their respective advantages. Mutations may distinguish ctDNA from healthy sources of cell-free DNA due to their irreversible disposition, provided that appropriate error suppression tools are employed and any contribution of mutations from clonal hematopoiesis is taken into account. DNA hypermethylation events potentially affect a larger number of recurrent genomic regions in cancer, contributing to their ability to inform the tumor-of-origin through cell-free DNA analysis. Moreover, hypermethylation events in the vicinity of cancer driver genes may influence their expression, thereby potentially reflecting cancer behavior and providing prognostic value. To date no study has utilized the combination of both mutation- and methylation-based methods for improved tumor-naïve detection and characterization of ctDNA in localized cancers.

Utilization of fluid-based biomarkers for prognostication, risk stratification, and disease surveillance may improve patient outcomes by guiding treatment decisions without the need for invasive tumor sampling. Although circulating tumor (ct)DNA in particular has shown promise as a liquid biopsy tool, in patients with low disease burden such as those with localized non-metastatic cancer, paired tumor profiling is often required. We hypothesized that multimodal analysis of genetic and epigenetic features from plasma cell-free DNA may enable broad applications of tumor-naïve ctDNA profiling. Mutation- and methylation-based profiling identified ctDNA in 65% of localized head and neck cancer patients. Results from both approaches were quantitative and strongly correlated, and their combined analysis revealed common features of tumor-derived DNA fragments. Moreover, ctDNA methylomes revealed tumor histology, putative prognostic biomarkers, and dynamic patterns of treatment response. These findings will aid future non-invasive biomarker discovery efforts and will inform clinical implementation of ctDNA for localized cancers.

Certain methods of capturing cell-free methylated DNA are described in Applicant's WO 2017/190215 and WO 2019/010564, both of which are incorporated by reference.

Specifically, we utilize both CAPP-Seq and cfMeDIP-seq to perform tumor-naïve ctDNA detection within a cohort of localized head and neck squamous cell carcinoma (HNSCC) patients. HNSCC is a clinically heterogenous disease that frequently recurs after definitive treatment and may benefit greatly from ctDNA detection to better inform treatment decisions and disease management³³. We demonstrate that utilization of both methods in parallel, as well as matched PBL-profiling, may achieve high-confidence tumor-naïve ctDNA detection. Furthermore, we show that the combined analysis reveals common molecular features of tumor-derived DNA fragments. Finally, we show that ctDNA methylomes revealed tumor histology, putative prognostic biomarkers, and dynamic patterns of treatment response, providing a blueprint for future biomarker studies in other disease settings

In an aspect, there is provided a method of detecting the presence of ctDNA from cancer cells in a subject comprising:
(a) providing a sample of cell-free DNA from a subject;
(b) subjecting the sample to library preparation to permit subsequent sequencing of the cell-free methylated DNA;
(c) optionally adding a first amount of filler DNA to the sample, wherein at least a portion of the filler DNA is methylated, then further optionally denaturing the sample;
(d) capturing cell-free methylated DNA using a binder selective for methylated polynucleotides;
(e) sequencing the captured cell-free methylated DNA;
(f) comparing the sequences of the captured cell-free methylated DNA to control cell-free methylated DNAs sequences from healthy and cancerous individuals;
(g) identifying the presence of DNA from cancer cells if there is a statistically significant similarity between one or more sequences of the captured cell-free methylated DNA and cell-free methylated DNAs sequences from cancerous individuals;
wherein in at least one of the capturing step, the comparing step or the identifying step, the subject cell-free methylated DNA is limited to a sub-population according to a fragment length metric.

Various sequencing techniques are known to the person skilled in the art, such as polymerase chain reaction (PCR) followed by Sanger sequencing. Also available are next-generation sequencing (NGS) techniques, also known as high-throughput sequencing, which includes various sequencing technologies including: Illumina (Solexa) sequencing, Roche 454 sequencing, Ion torrent: Proton/PGM sequencing, SOLiD sequencing, long reads sequencing (Oxford Nanopore and Pactbio). NGS allow for the sequencing of DNA and RNA much more quickly and cheaply than the previously used Sanger sequencing. In some embodiments, said sequencing is optimized for short read sequencing.

The term “subject” as used herein refers to any member of the animal kingdom. Thus, the methods and described herein are applicable to both human and veterinary disease and animal models. Preferred subjects are “patients,” i.e., living humans that are being investigated to determine whether treatment or medical care is needed for a disease or condition; or that are receiving medical care for a disease or condition (e.g., cancer).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The term “nucleic acid” used herein refers to a polynucleotide comprising two or more nucleotides, i.e., a polymeric form of nucleotides of any length, either deoxyribonucleotides (dNTPs) or ribonucleotides (rNTPs), or analogs thereof. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent. A “variant” nucleic acid is a polynucleotide having a nucleotide sequence identical to that of its original nucleic acid except having at least one nucleotide modified, for example, deleted, inserted, or replaced, respectively. The variant may have a nucleotide sequence at least about 80%, 90%, 95%, or 99%, identity to the nucleotide sequence of the original nucleic acid.

Cell-free methylated DNA is DNA that is circulating freely in the blood stream, and are methylated at various regions of the DNA. Samples, for example, plasma samples may be taken to analyze cell-free methylated DNA. Studies reveal that much of the circulating nucleic acids in blood arise from necrotic or apoptotic cells and greatly elevated levels of nucleic acids from apoptosis is observed in diseases such as cancer. Particularly for cancer, where the circulating DNA bears hallmark signs of the disease including mutations in oncogenes, microsatellite alterations, and, for certain cancers, viral genomic sequences, DNA or RNA in plasma has become increasingly studied as a potential biomarker for disease. For example, a quantitative assay for low levels of circulating tumor DNA in total circulating DNA may serve as a better marker for detecting the relapse of colorectal cancer compared with carcinoembryonic antigen, the standard biomarker used clinically. The circulating cfDNA may comprise circulating tumor DNA (ctDNA).

As used herein, “library preparation” includes list end-repair, A-tailing, adapter ligation, or any other preparation performed on the cell free DNA to permit subsequent sequencing of DNA.

As used herein, “filler DNA” may be noncoding DNA or it may consist of amplicons.

In some embodiments, the fragment length metric is fragment length. In some preferable embodiments, the subject cell-free methylated DNA is limited to fragments having a length of <170 bp, <165 bp, <160 bp, <155 bp, <150 bp, <145 bp, <140 bp, <135 bp, <130 bp, <125 bp, <120 bp, <115 bp, <110 bp, <105 bp, or <100 bp. In other preferable embodiments, the subject cell-free methylated DNA is limited to fragments having a length of between about 100-about 150 bp, 110-140 bp, or 120-130 bp.

In some embodiments, the fragment length metric is the fragment length distribution of the subject cell-free methylated DNA. In some preferable embodiments, the subject cell-free methylated DNA is limited to fragments within the bottom 50^{th}, 45^{th}, 40^{th}, 35^{th}, 30^{th}, 25^{th}, 20^{th}, 15^{th} or 10^{th} percentile based on length.

In some embodiments, the subject cell-free methylated DNA is further limited to fragments within Differentially Methylated Regions (DMRs).

In some embodiments, the limiting of the subject cell-free methylated DNA is during the capturing step.

In some embodiments, the limiting of the subject cell-free methylated DNA is during the comparing step.

In some embodiments, the limiting of the subject cell-free methylated DNA is during the identifying step.

In some embodiments, the comparison step is based on fit using a statistical classifier. Statistical classifiers using DNA methylation data may be used for assigning a sample to a particular disease state, such as cancer type or subtype. For the purpose of cancer type or subtype classification, a classifier would consist of one or more DNA methylation variables (i.e., features) within a statistical model, and the output of the statistical model would have one or more threshold values to distinguish between distinct disease states. The particular feature(s) and threshold value(s) that are used in the statistical classifier may be derived from prior knowledge of the cancer types or subtypes, from prior knowledge of the features that are likely to be most informative, from machine learning, or from a combination of two or more of these approaches.

In some embodiments, the classifier is machine learning-derived. Preferably, the classifier is an elastic net classifier, lasso, support vector machine, random forest, or neural network.

The genomic space that is analyzed may be genome-wide, or preferably restricted to regulatory regions (i.e., FANTOM5 enhancers, CpG Islands, CpG shores and CpG Shelves).

Preferably, the percentage of spike-in methylated DNA recovered is included as a covariate to control for pulldown efficiency variation.

For a classifier capable of distinguishing multiple cancer types (or subtypes) from one another, the classifier would preferably consist of differentially methylated regions from pairwise comparisons of each type (or subtype) of interest.

In some embodiments, the control cell-free methylated DNAs sequences from healthy and cancerous individuals are comprised in a database of Differentially Methylated Regions (DMRs) between healthy and cancerous individuals.

In some embodiments, the control cell-free methylated DNA sequences from healthy and cancerous individuals are limited to those control cell-free methylated DNA sequences which are differentially methylated as between healthy and cancerous individuals in DNA derived from cell-free DNA from bodily fluids, such as from blood serum, cerebral spinal fluid, urine stool, sputum, pleural fluid, ascites, tears, sweat, pap smear fluid, endoscopy brushings fluid, . . . etc., preferably from blood plasma.

Samples

A sample can be any biological sample isolated from a subject. For example, a sample may comprise, without limitation, bodily fluid, whole blood, platelets, serum, plasma, stool, red blood cells, white blood cells or leukocytes, endothelial cells, tissue biopsies, synovial fluid, lymphatic fluid, ascites fluid, interstitial or extracellular fluid, the fluid in spaces between cells, including gingival crevicular fluid, bone marrow, cerebrospinal fluid, saliva, mucous, sputum, semen, sweat, urine, fluid from nasal brushings, fluid from a pap smear, or any other bodily fluids. A bodily fluid may include saliva, blood, or serum. A sample may also be a tumor sample, which may be obtained from a subject by various approaches, including, but not limited to, venipuncture, excretion, ejaculation, massage, biopsy, needle aspirate, lavage, scraping, surgical incision, or intervention or other approaches. A sample may be a cell-free sample (e.g., substantially free of cells). DNA samples may be denatured, for example, using sufficient heat.

In some embodiments, the present disclosure provides a system, method, or kit that includes or uses one or more biological samples. The one or more samples used herein may comprise any substance containing or presumed to contain nucleic acids. A sample may include a biological sample obtained from a subject. In some embodiments, a biological sample is a liquid sample.

In some embodiments, the sample comprises less than about 100 ng, 90 ng, 80 ng, 75 ng, 70 ng, 60 ng, 50 ng, 40 ng, 30 ng, 20 ng, 10 ng, 5 ng, 1 ng or any amount in between the numbers of cell-free nucleic acid molecules. Further, in some embodiments, the sample comprises less than about 1 pg, less than about 5 pg, less than about 10 pg, less than about 20 pg, less than about 30 pg, less than about 40 pg, less than about 50 pg, less than about 100 pg, less than about 200 pg, less than about 500 pg, less than about 1 ng, less than about 5 ng, less than about 10 ng, less than about 20 ng, less than about 30 ng, less than about 40 ng, less than about 50 ng, less than about 100 ng, less than about 200 ng, less than about 500 ng, less than about 1000 ng, or any amount in between the numbers of cell-free nucleic acid molecules.

In some embodiments, the present disclosure comprises methods and systems for filling in the sample with a amount of filler DNA to generate a mixture sample, wherein the mixture sample comprises at least about 50 ng, 55 ng, 60 ng, 65 ng, 70 ng, 75 ng, 80 ng, 85 ng, 90 ng, 95 ng, 100 ng, 120 ng, 140 ng, 160 ng, 180 ng, 200 ng, or any amount in between the numbers of the total amount of the nucleic acid mixture. In some embodiments, the filler DNA comprises at least about 5%, 10%, 15%, 20%, 30%, 40%, 50%, 60%, 70%, 80%, 90%, or 100% methylated filler DNA with remainder being unmethylated filler DNA, and preferably between 5% and 50%, between 10%-40%, or between 15%-30% methylated filler DNA. In some embodiments, the mixture sample comprise an amount of filler DNA from 20 ng to 100 ng, preferably 30 ng to 100 ng, more preferably 50 ng to 100 ng. In some embodiments, the cell-free DNA from the sample and the first amount of filler DNA together comprises at least 50 ng of total DNA, preferably at least 100 ng of total DNA.

In some embodiments, the filler DNA is 50 bp to 800 bp long, preferably 100 bp to 600 bp long, and more preferably 200 bp to 600 bp long. In some embodiments, the filler DNA is double stranded. The filler DNA is double stranded. For example, the filler DNA can be junk DNA. The filler DNA may also be endogenous or exogenous DNA. For example, the filler DNA is non-human DNA, and in preferred embodiments, λ DNA. As used herein, “λ DNA” refers to Enterobacteria phage λ DNA. In some embodiments, the filler DNA has no alignment to human DNA.

In some embodiments, the sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample may be taken at a first time point and sequenced, and then another sample may be taken at a subsequent time point and sequenced. Such methods may be used, for example, for longitudinal monitoring purposes to track the development or progression of a disease. In some embodiments, the progression of a disease may be tracked before treatment, after treatment, or during the course of treatment, to determine the treatment's effectiveness. For example, a method as described herein may be performed on a subject prior to, and after, a medical treatment to measure the disease's progression or regression in response to the medical treatment.

After obtaining a sample from the subject, the sample may be processed to generate datasets indicative of a disease or disorder of the subject. For example, a presence, absence, or quantitative assessment of cell-free nucleic acid molecules (e.g., ctDNA molecules) of the sample at a panel of cancer-associated genomic loci or microbiome-associated loci may be indicative of a cancer of the subject. Processing the sample obtained from the subject may comprise (i) subjecting the sample to conditions that are sufficient to isolate, enrich, or extract a plurality of cell-free nucleic acid molecules, and (ii) assaying the plurality of cell-free nucleic acid molecules to generate the dataset (e.g., nucleic acid sequences). In some embodiments, a plurality of cell-free nucleic acid molecules is extracted from the sample and subjected to sequencing to generate a plurality of sequencing reads.

In some embodiments, the cell-free nucleic acid molecules may comprise cell-free ribonucleic acid (cfRNA) or cell-free deoxyribonucleic acid (cfDNA). The cell-free nucleic acid molecules (e.g., cfRNA or cfDNA) may be extracted from the sample by a variety of methods. The cell-free nucleic acid molecule may be enriched by a plurality of probes configured to enrich nucleic acid (e.g., RNA or DNA) molecules corresponding to a panel of cancer-associated genomic loci. The probes may have sequence complementarity with nucleic acid sequences from one or more of the panel of cancer-associated genomic loci. The panel of cancer-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct cancer-associated genomic loci. The probes may be nucleic acid molecules (e.g., RNA or DNA) having sequence complementarity with nucleic acid sequences (e.g., RNA or DNA) of the one or more genomic loci (e.g., cancer-associated genomic loci). These nucleic acid molecules may be primers or enrichment sequences. The assaying of the sample using probes that are selective for the one or more genomic loci (e.g., cancer-associated genomic loci or microbiome-associated loci) may comprise use of array hybridization, polymerase chain reaction (PCR), or nucleic acid sequencing (e.g., RNA sequencing or DNA sequencing).

Nucleic Acid Molecules Sequencing

The present disclosure provides methods and technologies for determining the sequence of nucleotide bases in one or more polynucleotides. The polynucleotides may be, for example, nucleic acid molecules such as deoxyribonucleic acid (DNA) or ribonucleic acid (RNA), including variants or derivatives thereof (e.g., single stranded DNA). Sequencing may be performed by various systems currently available, such as, without limitation, a sequencing system by Illumina®, Pacific Biosciences (PacBio®), Oxford Nanopore®, or Life Technologies (Ion Torrent®). Further, any sequencing methods that provides fragment length such as pair-end sequencing may be utilized. Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

In some embodiments, the sequencing reads are obtained via a next-generation sequencing method or a next-next-generation sequencing method. In some embodiments, the sequencing methods comprises CAncer Personalized Profiling by deep Sequencing (CAPP-Seq), which is a next-generation sequencing based method used to quantify circulating DNA in cancer (ctDNA). This method may be generalized for any cancer type that is known to have recurrent mutations and may detect one molecule of mutant DNA in 10,000 molecules of healthy DNA. In some embodiments, the sequencing methods comprise cfMeDIP sequencing as described by Shen et al., sensitive tumor detection and classification using plasma cell-free DNA methylomes, (2018) Nature, which is incorporated herein in its entirety. In some embodiments, the sequencing comprises bisulfite sequencing.

In some embodiments, sequencing comprises modification of a nucleic acid molecule or fragment thereof, for example, by ligating a barcode, a unique molecular identifier (UMI), or anothertag to the nucleic acid molecule or fragment thereof. Ligating a barcode, UMI, or tag to one end of a nucleic acid molecule or fragment thereof may facilitate analysis of the nucleic acid molecule or fragment thereof following sequencing. In some embodiments, a barcode is a unique barcode (e.g., a UMI). In some embodiments, a barcode is non-unique, and barcode sequences may be used in connection with endogenous sequence information such as the start and stop sequences of a target nucleic acid (e.g., the target nucleic acid is flanked by the barcode and the barcode sequences, in connection with the sequences at the beginning and end of the target nucleic acid, creates a uniquely tagged molecule). A barcode, UMI, or tag may be a known sequence used to associate a polynucleotide or fragment thereof with an input or target nucleic acid molecule or fragment thereof. A barcode, UMI, or tag may comprise natural nucleotides or non-natural (e.g., modified) nucleotides (e.g., as described herein). A barcode sequence may be contained within an adapter sequence such that the barcode sequence may be contained within a sequencing read. A barcode sequence may comprise at least 4, 5, 6, 7, 8, 9, 10, 11, 12, 13, 14, 15, 16, or more nucleotides in length. In some cases, a barcode sequence may be of sufficient length and may be sufficiently different from another barcode sequence to allow the identification of a sample based on a barcode sequence with which it is associated. A barcode sequence, or a combination of barcode sequences, may be used to tag and subsequently identify an “original” nucleic acid molecule or fragment thereof (e.g., a nucleic acid molecule or fragment thereof present in a sample from a subject). In some cases, a barcode sequence, or a combination of barcode sequences, is used in conjunction with endogenous sequence information to identify an original nucleic acid molecule or fragment thereof. For example, a barcode sequence, or a combination of barcode sequences, may be used with endogenous sequences adjacent to a barcode, UMI, or tag (e.g., the beginning and end of the endogenous sequences).

Processing a nucleic acid molecule or fragment thereof may comprise performing nucleic acid amplification. For example, any type of nucleic acid amplification reaction may be used to amplify a target nucleic acid molecule or fragment thereof and generate an amplified product. Non-limiting examples of nucleic acid amplification methods include reverse transcription, primer extension, polymerase chain reaction (PCR), ligase chain reaction, asymmetric amplification, rolling circle amplification, and multiple displacement amplification (MDA). Examples of PCR include, but are not limited to, quantitative PCR, real-time PCR, digital PCR, emulsion PCR, hot start PCR, multiplex PCR, asymmetric PCR, nested PCR, and assembly PCR. Nucleic acid amplification may involve one or more reagents such as one or more primers, probes, polymerases, buffers, enzymes, and deoxyribonucleotides. Nucleic acid amplification may be isothermal or may comprise thermal cycling. and/or with the length of the endogenous sequence.

Methylation Profile

The present disclosure provides methods, systems, and kits for producing a methylation profile of a subject that has a disease/condition or is suspected of having such disease/condition, wherein the methylation profile may be used to determine whether the subject has the disease/condition or is at risk of having the disease/condition. Before using cfMeDIP-seq, the samples disclosed herein are subjected to library preparation. In short, after end-repair and A-tailing, the samples are ligated to nucleic acid adapters and digested using enzymes. As described above under the sample section, the prepared libraries may be combined with filler nucleic acids (e.g., filler λ DNAs) to minimize the effect of low abundance ctDNA in the prepared libraries and generate mixed samples. In some embodiments, when the disease/condition is a locoregionally (non-metastatic) cancer, the amount of ctDNA is low and may not be easily and accurately measured and quantified. The mixed samples are brought to at least about 50 ng, 80 ng, 100 ng, 120 ng, 150 ng, or 200 ng and are subjected to further enrichment.

The methods, system, and kits described herein are applicable to a wide variety of cancers, including but not limited to adrenal cancer, anal cancer, bile duct cancer, bladder cancer, bone cancer, brain/cns tumors, breast cancer, castleman disease, cervical cancer, colon/rectum cancer, endometrial cancer, esophagus cancer, ewing family of tumors, eye cancer, gallbladder cancer, gastrointestinal carcinoid tumors, gastrointestinal stromal tumor (gist), gestational trophoblastic disease, hodgkin disease, kaposi sarcoma, kidney cancer, laryngeal and hypopharyngeal cancer, leukemia (acute lymphocytic, acute myeloid, chronic lymphocytic, chronic myeloid, chronic myelomonocytic), liver cancer, lung cancer (non-small cell, small cell, lung carcinoid tumor), lymphoma, lymphoma of the skin, malignant mesothelioma, multiple myeloma, myelodysplastic syndrome, nasal cavity and paranasal sinus cancer, nasopharyngeal cancer, neuroblastoma, non-hodgkin lymphoma, oral cavity and oropharyngeal cancer, osteosarcoma, ovarian cancer, penile cancer, pituitary tumors, prostate cancer, retinoblastoma, rhabdomyosarcoma, salivary gland cancer, sarcoma—adult soft tissue cancer, skin cancer (basal and squamous cell, melanoma, merkel cell), small intestine cancer, stomach cancer, testicular cancer, thymus cancer, thyroid cancer, uterine sarcoma, vaginal cancer, vulvar cancer, waldenstrom macroglobulinemia, wilms tumor. In an embodiment, the cancer is head and neck squamous cell carcinoma.

A binder may be used to enrich the mixed samples. In some embodiments, the binder is a protein comprising a Methyl-CpG-binding domain. One such exemplary protein is MBD2 protein. As used herein, “Methyl-CpG-binding domain (MBD)” refers to certain domains of proteins and enzymes that is approximately 70 residues long and binds to DNA that contains one or more symmetrically methylated CpGs. The MBD of MeCP2, MBD1, MBD2, MBD4 and BAZ2 mediates binding to DNA, and in cases of MeCP2, MBD1 and MBD2, preferentially to methylated CpG. Human proteins MECP2, MBD1, MBD2, MBD3, and MBD4 comprise a family of nuclear proteins related by the presence in each of a methyl-CpG-binding domain (MBD). Each of these proteins, with the exception of MBD3, is capable of binding specifically to methylated DNA.

In other embodiments, the binder is an antibody and capturing cell-free methylated DNA comprises immunoprecipitating the cell-free methylated DNA using the antibody. As used herein, “immunoprecipitation” refers a technique of precipitating an antigen (such as polypeptides and nucleotides) out of solution using an antibody that specifically binds to that particular antigen. This process may be used to isolate and concentrate a particular protein or DNA from a sample and requires that the antibody be coupled to a solid substrate at some point in the procedure. The solid substrate includes for examples beads, such as magnetic beads. Other types of beads and solid substrates may be used.

One exemplary antibody is 5-MeC antibody. For the immunoprecipitation procedure, in some embodiments at least 0.05 μg of the antibody is added to the sample; while in more preferred embodiments at least 0.16 μg of the antibody is added to the sample. To confirm the immunoprecipitation reaction, in some embodiments the method described herein further comprises the step of adding a second amount of control DNA to the sample.

The enriched samples are further amplified, purified, and sequenced to generate a plurality of sequence reads. The plurality of sequence reads is analyzed to identify a plurality of Differentially Methylated Regions (DMRs). In some embodiments, the plurality of DMRs comprises DMRs derived from cell free nucleic acid molecules that are derived from peripheral blood leukocytes (PBLs). In some embodiments, the plurality of DMRs comprises at least about 750,000 non-overlapping about 300-bp nucleic acid fragment window. These fragments comprise greater than or equal to 8 CpG islands. In some embodiments, DMRs are identified from comparing sequence reads generated from samples obtained from patients with the disease/condition to sequence reads generated from samples obtained from healthy controls. In some embodiments, the healthy controls comprise a same set of risk factors for developing the disease/condition. In some embodiments, the plurality of DMRs comprises at least about 997 DMRs: about 941 hypermethylated in HNSCC and 56 hypomethylated in HNSCC (Table 5). Using the same disclosed approach here, hypermethylated DMRs may be detected for a different cancer (e.g., lung cancer, pancreatic cancer, colorectal cancer) and hypomethylated DMRs may be detected for the different cancer.

TABLE 5
A list of ctDNA derived DMRs

| | | DMR |
|---|---|---|
| windowPos (Genomic | ensemblId Gene ID (a | methylation |
| position of each DMR) | DMR related to a gene) | level |
| chr1.50881501.50881800 | ENSG00000142700 | hyper |
| chr1.50881801.50882100 | ENSG00000142700 | hyper |
| chr1.63786301.63786600 | ENSG00000230798 | hyper |
| chr1.119527501.119527800 | ENSG00000092607 | hyper |
| chr1.119550601.119550900 | ENSG00000239216 | hyper |
| chr1.148603801.148604100 | ENSG00000207205 | hyper |
| chr1.149155501.149155800 | ENSG00000202167 | hyper |
| chr1.149223301.149223600 | ENSG00000206737 | hyper |
| chr1.149223601.149223900 | ENSG00000206737 | hyper |
| chr1.17216101.17216400 | ENSG00000058453 | hyper |
| chr1.91182301.91182600 | ENSG00000143032 | hyper |
| chr1.98511601.98511900 | ENSG00000225206 | hyper |
| chr1.99470101.99470400 | ENSG00000117598 | hyper |
| chr1.145944901.145945200 | ENSG00000201105 | hyper |
| chr1.147486601.147486900 | ENSG00000206791 | hyper |
| chr1.148598101.148598400 | ENSG00000237253 | hyper |
| chr1.148760401.148760700 | ENSG00000237343 | hyper |
| chr1.149223901.149224200 | ENSG00000206737 | hyper |
| chr1.149224201.149224500 | ENSG00000206737 | hyper |
| chr1.17215801.17216100 | ENSG00000058453 | hyper |
| chr1.20810101.20810400 | ENSG00000162545 | hyper |
| chr1.26551801.26552100 | ENSG00000236155 | hyper |
| chr1.50893501.50893800 | ENSG00000142700 | hyper |
| chr1.57888301.57888600 | ENSG00000173406 | hyper |
| chr1.63785401.63785700 | ENSG00000230798 | hyper |
| chr1.63786001.63786300 | ENSG00000230798 | hyper |
| chr1.66258301.66258600 | ENSG00000184588 | hyper |
| chr1.75595801.75596100 | ENSG00000224127 | hyper |
| chr1.77334601.77334900 | ENSG00000117069 | hyper |
| chr1.91182601.91182900 | ENSG00000143032 | hyper |
| chr1.91183801.91184100 | ENSG00000143032 | hyper |
| chr1.92948101.92948400 | ENSG00000162676 | hyper |
| chr1.98511301.98511600 | ENSG00000225206 | hyper |
| chr1.99469801.99470100 | ENSG00000117598 | hyper |
| chr1.110612401.110612700 | ENSG00000143093 | hyper |
| chr1.111216901.111217200 | ENSG00000177272 | hyper |
| chr1.111506101.111506400 | ENSG00000121931 | hyper |
| chr1.119526601.119526900 | ENSG00000092607 | hyper |
| chr1.119526901.119527200 | ENSG00000092607 | hyper |
| chr1.119527201.119527500 | ENSG00000092607 | hyper |
| chr1.119532601.119532900 | ENSG00000092607 | hyper |
| chr1.119536201.119536500 | ENSG00000092607 | hyper |
| chr1.119543101.119543400 | ENSG00000226172 | hyper |
| chr1.119550901.119551200 | ENSG00000239216 | hyper |
| chr1.119551201.119551500 | ENSG00000239216 | hyper |
| chr1.145944601.145944900 | ENSG00000201105 | hyper |
| chr1.145963501.145963800 | ENSG00000207418 | hyper |
| chr1.145979401.145979700 | ENSG00000207418 | hyper |
| chr1.145990801.145991100 | ENSG00000229828 | hyper |
| chr1.147486301.147486600 | ENSG00000206791 | hyper |
| chr1.147505201.147505500 | ENSG00000206585 | hyper |
| chr1.147521101.147521400 | ENSG00000206585 | hyper |
| chr1.147752701.147753000 | ENSG00000234283 | hyper |
| chr1.147753001.147753300 | ENSG00000234283 | hyper |
| chr1.147775201.147775500 | ENSG00000238107 | hyper |
| chr1.147790501.147790800 | ENSG00000235988 | hyper |
| chr1.149156101.149156400 | ENSG00000202167 | hyper |
| chr1.149156401.149156700 | ENSG00000202167 | hyper |
| chr1.149224501.149224800 | ENSG00000206737 | hyper |
| chr1.149400001.149400300 | ENSG00000273213 | hyper |
| chr1.149719501.149719800 | ENSG00000234232 | hyper |
| chr1.242687101.242687400 | ENSG00000180287 | hyper |
| chr1.165323701.165324000 | ENSG00000162761 | hyper |
| chr1.177140401.177140700 | ENSG00000198797 | hyper |
| chr1.207999301.207999600 | ENSG00000203709 | hyper |
| chr1.217311301.217311600 | ENSG00000196482 | hyper |
| chr1.234041101.234041400 | ENSG00000183780 | hyper |
| chr1.237204901.237205200 | ENSG00000198626 | hyper |
| chr1.240255001.240255300 | ENSG00000155816 | hyper |
| chr2.19558501.19558800 | ENSG00000143867 | hyper |
| chr1.161039401.161039700 | ENSG00000186517 | hyper |
| chr1.165321601.165321900 | ENSG00000162761 | hyper |
| chr1.165323401.165323700 | ENSG00000162761 | hyper |
| chr1.165324301.165324600 | ENSG00000162761 | hyper |
| chr1.165324601.165324900 | ENSG00000162761 | hyper |
| chr1.167090701.167091000 | ENSG00000198842 | hyper |
| chr1.167682601.167682900 | ENSG00000198771 | hyper |
| chr1.169396501.169396800 | ENSG00000117477 | hyper |
| chr1.169396801.169397100 | ENSG00000117477 | hyper |
| chr1.170630101.170630400 | ENSG00000116132 | hyper |
| chr1.173638801.173639100 | ENSG00000183831 | hyper |
| chr1.180203701.180204000 | ENSG00000121454 | hyper |
| chr1.180204001.180204300 | ENSG00000121454 | hyper |
| chr1.180204301.180204600 | ENSG00000121454 | hyper |
| chr1.200010001.200010300 | ENSG00000116833 | hyper |
| chr1.200011201.200011500 | ENSG00000116833 | hyper |
| chr1.214159501.214159800 | ENSG00000230461 | hyper |
| chr1.217307401.217307700 | ENSG00000196482 | hyper |
| chr1.217307701.217308000 | ENSG00000196482 | hyper |
| chr1.217308001.217308300 | ENSG00000196482 | hyper |
| chr1.217309501.217309800 | ENSG00000196482 | hyper |
| chr1.217309801.217310100 | ENSG00000196482 | hyper |
| chr1.217310101.217310400 | ENSG00000196482 | hyper |
| chr1.217311601.217311900 | ENSG00000196482 | hyper |
| chr1.217313101.217313400 | ENSG00000196482 | hyper |
| chr1.217313401.217313700 | ENSG00000196482 | hyper |
| chr1.220959601.220959900 | ENSG00000186205 | hyper |
| chr1.224804401.224804700 | ENSG00000143786 | hyper |
| chr1.224804701.224805000 | ENSG00000143786 | hyper |
| chr1.228652201.228652500 | ENSG00000181201 | hyper |
| chr1.235814101.235814400 | ENSG00000168243 | hyper |
| chr1.239550601.239550900 | ENSG00000133019 | hyper |
| chr1.239550901.239551200 | ENSG00000133019 | hyper |
| chr1.239551201.239551500 | ENSG00000133019 | hyper |
| chr1.242686801.242687100 | ENSG00000180287 | hyper |
| chr2.1746901.1747200 | ENSG00000130508 | hyper |
| chr2.5830801.5831100 | ENSG00000224128 | hyper |
| chr2.5831101.5831400 | ENSG00000224128 | hyper |
| chr2.19555801.19556100 | ENSG00000143867 | hyper |
| chr2.45155101.45155400 | ENSG00000259439 | hyper |
| chr2.45159301.45159600 | ENSG00000259439 | hyper |
| chr2.45160201.45160500 | ENSG00000259439 | hyper |
| chr2.45170101.45170400 | ENSG00000138083 | hyper |
| chr2.45171301.45171600 | ENSG00000138083 | hyper |
| chr2.45228301.45228600 | ENSG00000170577 | hyper |
| chr2.45228601.45228900 | ENSG00000170577 | hyper |
| chr2.45231301.45231600 | ENSG00000170577 | hyper |
| chr2.45231901.45232200 | ENSG00000170577 | hyper |
| chr2.45233401.45233700 | ENSG00000170577 | hyper |
| chr2.50574301.50574600 | ENSG00000179915 | hyper |
| chr2.85107301.85107600 | ENSG00000186854 | hyper |
| chr2.119600401.119600700 | ENSG00000163064 | hyper |
| chr2.119607601.119607900 | ENSG00000163064 | hyper |
| chr2.176933401.176933700 | ENSG00000174279 | hyper |
| chr2.63280801.63281100 | ENSG00000115507 | hyper |
| chr2.80531701.80532000 | ENSG00000066032 | hyper |
| chr2.115920601.115920900 | ENSG00000175497 | hyper |
| chr2.131721901.131722200 | ENSG00000136002 | hyper |
| chr2.177030001.177030300 | ENSG00000128652 | hyper |
| chr2.63279001.63279300 | ENSG00000115507 | hyper |
| chr2.63279901.63280200 | ENSG00000115507 | hyper |
| chr2.63280201.63280500 | ENSG00000115507 | hyper |
| chr2.63280501.63280800 | ENSG00000115507 | hyper |
| chr2.63281101.63281400 | ENSG00000115507 | hyper |
| chr2.63281401.63281700 | ENSG00000115507 | hyper |
| chr2.63285301.63285600 | ENSG00000115507 | hyper |
| chr2.63285601.63285900 | ENSG00000115507 | hyper |
| chr2.71017201.71017500 | ENSG00000183733 | hyper |
| chr2.73147201.73147500 | ENSG00000135638 | hyper |
| chr2.73519501.73519800 | ENSG00000135625 | hyper |
| chr2.80529901.80530200 | ENSG00000066032 | hyper |
| chr2.80530201.80530500 | ENSG00000066032 | hyper |
| chr2.84743401.84743700 | ENSG00000115423 | hyper |
| chr2.85107001.85107300 | ENSG00000186854 | hyper |
| chr2.111876901.111877200 | ENSG00000153094 | hyper |
| chr2.119600701.119601000 | ENSG00000163064 | hyper |
| chr2.119614501.119614800 | ENSG00000163064 | hyper |
| chr2.119614801.119615100 | ENSG00000163064 | hyper |
| chr2.119616301.119616600 | ENSG00000163064 | hyper |
| chr2.119616601.119616900 | ENSG00000163064 | hyper |
| chr2.124782301.124782600 | ENSG00000228400 | hyper |
| chr2.139537201.139537500 | ENSG00000144227 | hyper |
| chr2.149645701.149646000 | ENSG00000231079 | hyper |
| chr2.157176601.157176900 | ENSG00000153234 | hyper |
| chr2.168150001.168150300 | ENSG00000228222 | hyper |
| chr2.172946101.172946400 | ENSG00000172878 | hyper |
| chr2.172952401.172952700 | ENSG00000144355 | hyper |
| chr2.173099701.173100000 | ENSG00000232555 | hyper |
| chr2.173100001.173100300 | ENSG00000232555 | hyper |
| chr2.175191901.175192200 | ENSG00000231453 | hyper |
| chr2.175193401.175193700 | ENSG00000231453 | hyper |
| chr2.175193701.175194000 | ENSG00000231453 | hyper |
| chr2.175205701.175206000 | ENSG00000217236 | hyper |
| chr2.176931601.176931900 | ENSG00000174279 | hyper |
| chr2.176931901.176932200 | ENSG00000174279 | hyper |
| chr2.176933101.176933400 | ENSG00000174279 | hyper |
| chr2.176936401.176936700 | ENSG00000174279 | hyper |
| chr2.176943301.176943600 | ENSG00000174279 | hyper |
| chr2.176946601.176946900 | ENSG00000174279 | hyper |
| chr2.176947201.176947500 | ENSG00000174279 | hyper |
| chr2.176948101.176948400 | ENSG00000174279 | hyper |
| chr2.176964901.176965200 | ENSG00000170178 | hyper |
| chr2.176965201.176965500 | ENSG00000170178 | hyper |
| chr2.176976901.176977200 | ENSG00000128710 | hyper |
| chr2.176981101.176981400 | ENSG00000128710 | hyper |
| chr2.177054301.177054600 | ENSG00000128645 | hyper |
| chr2.177054601.177054900 | ENSG00000128645 | hyper |
| chr2.182322601.182322900 | ENSG00000115232 | hyper |
| chr2.200333701.200334000 | ENSG00000119042 | hyper |
| chr2.200334001.200334300 | ENSG00000119042 | hyper |
| chr3.27770401.27770700 | ENSG00000163508 | hyper |
| chr3.62353801.62354100 | ENSG00000241472 | hyper |
| chr2.223161901.223162200 | ENSG00000135903 | hyper |
| chr2.223162801.223163100 | ENSG00000135903 | hyper |
| chr2.223166401.223166700 | ENSG00000163081 | hyper |
| chr2.223176301.223176600 | ENSG00000267034 | hyper |
| chr2.229046101.229046400 | ENSG00000153820 | hyper |
| chr2.237072601.237072900 | ENSG00000168505 | hyper |
| chr2.237082201.237082500 | ENSG00000233611 | hyper |
| chr3.27765001.27765300 | ENSG00000163508 | hyper |
| chr3.27765301.27765600 | ENSG00000163508 | hyper |
| chr3.62353501.62353800 | ENSG00000241472 | hyper |
| chr3.192126001.192126300 | ENSG00000114279 | hyper |
| chr4.4868101.4868400 | ENSG00000163132 | hyper |
| chr4.13532401.13532700 | ENSG00000109705 | hyper |
| chr4.13532701.13533000 | ENSG00000109705 | hyper |
| chr3.169377901.169378200 | ENSG00000085276 | hyper |
| chr3.170137201.170137500 | ENSG00000013297 | hyper |
| chr3.194409001.194409300 | ENSG00000185112 | hyper |
| chr3.128210701.128211000 | ENSG00000179348 | hyper |
| chr3.129693901.129694200 | ENSG00000170893 | hyper |
| chr3.129694201.129694500 | ENSG00000170893 | hyper |
| chr3.137480101.137480400 | ENSG00000168875 | hyper |
| chr3.138657301.138657600 | ENSG00000244578 | hyper |
| chr3.138657901.138658200 | ENSG00000244578 | hyper |
| chr3.147077401.147077700 | ENSG00000243620 | hyper |
| chr3.147105901.147106200 | ENSG00000174963 | hyper |
| chr3.147109501.147109800 | ENSG00000174963 | hyper |
| chr3.147109801.147110100 | ENSG00000174963 | hyper |
| chr3.147110101.147110400 | ENSG00000174963 | hyper |
| chr3.147114301.147114600 | ENSG00000174963 | hyper |
| chr3.147124201.147124500 | ENSG00000174963 | hyper |
| chr3.157812601.157812900 | ENSG00000168779 | hyper |
| chr3.157821301.157821600 | ENSG00000168779 | hyper |
| chr3.159944401.159944700 | ENSG00000180044 | hyper |
| chr3.170136901.170137200 | ENSG00000013297 | hyper |
| chr3.173302801.173303100 | ENSG00000169760 | hyper |
| chr3.181422001.181422300 | ENSG00000242808 | hyper |
| chr3.181441501.181441800 | ENSG00000242808 | hyper |
| chr3.192126301.192126600 | ENSG00000114279 | hyper |
| chr3.192231901.192232200 | ENSG00000114279 | hyper |
| chr4.4856401.4856700 | ENSG00000273396 | hyper |
| chr4.9178201.9178500 | ENSG00000229924 | hyper |
| chr4.13533001.13533300 | ENSG00000109705 | hyper |
| chr4.20255701.20256000 | ENSG00000145147 | hyper |
| chr4.20256001.20256300 | ENSG00000145147 | hyper |
| chr4.37245601.37245900 | ENSG00000174145 | hyper |
| chr4.37245901.37246200 | ENSG00000174145 | hyper |
| chr4.41749501.41749800 | ENSG00000109132 | hyper |
| chr4.41875501.41875800 | ENSG00000245870 | hyper |
| chr4.42398701.42399000 | ENSG00000178343 | hyper |
| chr4.44449501.44449800 | ENSG00000183783 | hyper |
| chr4.54969901.54970200 | ENSG00000145216 | hyper |
| chr4.85402801.85403100 | ENSG00000163623 | hyper |
| chr5.2743201.2743500 | ENSG00000170561 | hyper |
| chr4.134071801.134072100 | ENSG00000138650 | hyper |
| chr4.174450001.174450300 | ENSG00000164107 | hyper |
| chr4.190938901.190939200 | ENSG00000201145 | hyper |
| chr4.81187501.81187800 | ENSG00000138675 | hyper |
| chr4.85414501.85414800 | ENSG00000163623 | hyper |
| chr4.85414801.85415100 | ENSG00000163623 | hyper |
| chr4.85417801.85418100 | ENSG00000163623 | hyper |
| chr4.85418101.85418400 | ENSG00000163623 | hyper |
| chr4.85418401.85418700 | ENSG00000163623 | hyper |
| chr4.104640901.104641200 | ENSG00000169836 | hyper |
| chr4.107956501.107956800 | ENSG00000155011 | hyper |
| chr4.110223601.110223900 | ENSG00000188517 | hyper |
| chr4.111533101.111533400 | ENSG00000250103 | hyper |
| chr4.111555301.111555600 | ENSG00000164093 | hyper |
| chr4.111562501.111562800 | ENSG00000164093 | hyper |
| chr4.121992301.121992600 | ENSG00000173376 | hyper |
| chr4.122686201.122686500 | ENSG00000164112 | hyper |
| chr4.134069401.134069700 | ENSG00000250241 | hyper |
| chr4.134071501.134071800 | ENSG00000138650 | hyper |
| chr4.134072101.134072400 | ENSG00000138650 | hyper |
| chr4.134072401.134072700 | ENSG00000138650 | hyper |
| chr4.134072701.134073000 | ENSG00000138650 | hyper |
| chr4.134073901.134074200 | ENSG00000138650 | hyper |
| chr4.144621301.144621600 | ENSG00000183090 | hyper |
| chr4.147561601.147561900 | ENSG00000151615 | hyper |
| chr4.158143201.158143500 | ENSG00000120251 | hyper |
| chr4.158143501.158143800 | ENSG00000120251 | hyper |
| chr4.172733701.172734000 | ENSG00000174473 | hyper |
| chr4.172734601.172734900 | ENSG00000174473 | hyper |
| chr4.174422101.174422400 | ENSG00000164107 | hyper |
| chr4.174427801.174428100 | ENSG00000164107 | hyper |
| chr4.174429601.174429900 | ENSG00000164107 | hyper |
| chr4.174430201.174430500 | ENSG00000164107 | hyper |
| chr4.174448501.174448800 | ENSG00000164107 | hyper |
| chr5.2754901.2755200 | ENSG00000186493 | hyper |
| chr5.3104701.3105000 | ENSG00000249808 | hyper |
| chr5.3116701.3117000 | ENSG00000249808 | hyper |
| chr5.3590701.3591000 | ENSG00000170549 | hyper |
| chr5.3599401.3599700 | ENSG00000170549 | hyper |
| chr5.3600601.3600900 | ENSG00000170549 | hyper |
| chr5.3602101.3602400 | ENSG00000170549 | hyper |
| chr5.54518701.54519000 | ENSG00000234602 | hyper |
| chr5.54519001.54519300 | ENSG00000234602 | hyper |
| chr5.122422501.122422800 | ENSG00000223652 | hyper |
| chr5.32712601.32712900 | ENSG00000113389 | hyper |
| chr5.40680901.40681200 | ENSG00000171522 | hyper |
| chr5.42994801.42995100 | ENSG00000271788 | hyper |
| chr5.42995101.42995400 | ENSG00000271788 | hyper |
| chr5.54519301.54519600 | ENSG00000234602 | hyper |
| chr5.57878101.57878400 | ENSG00000152932 | hyper |
| chr5.63257401.63257700 | ENSG00000248285 | hyper |
| chr5.72528901.72529200 | ENSG00000249743 | hyper |
| chr5.72529201.72529500 | ENSG00000249743 | hyper |
| chr5.72596701.72597000 | ENSG00000249743 | hyper |
| chr5.72740101.72740400 | ENSG00000251493 | hyper |
| chr5.72740401.72740700 | ENSG00000251493 | hyper |
| chr5.80256601.80256900 | ENSG00000251450 | hyper |
| chr5.94955701.94956000 | ENSG00000178015 | hyper |
| chr5.95768101.95768400 | ENSG00000251314 | hyper |
| chr5.95768701.95769000 | ENSG00000251314 | hyper |
| chr5.115152001.115152300 | ENSG00000129596 | hyper |
| chr5.115152301.115152600 | ENSG00000129596 | hyper |
| chr5.122423401.122423700 | ENSG00000223652 | hyper |
| chr5.134376301.134376600 | ENSG00000224186 | hyper |
| chr5.134825101.134825400 | ENSG00000249639 | hyper |
| chr5.134825401.134825700 | ENSG00000249639 | hyper |
| chr5.134826001.134826300 | ENSG00000249639 | hyper |
| chr5.140012101.140012400 | ENSG00000170458 | hyper |
| chr5.140012401.140012700 | ENSG00000170458 | hyper |
| chr5.140346601.140346900 | ENSG00000204970 | hyper |
| chr5.154026901.154027200 | ENSG00000221552 | hyper |
| chr5.172672201.172672500 | ENSG00000183072 | hyper |
| chr6.1378501.1378800 | ENSG00000261730 | hyper |
| chr6.10421701.10422000 | ENSG00000228478 | hyper |
| chr6.26721001.26721300 | ENSG00000261584 | hyper |
| chr6.26722501.26722800 | ENSG00000261584 | hyper |
| chr6.26722801.26723100 | ENSG00000261584 | hyper |
| chr6.26745301.26745600 | ENSG00000261584 | hyper |
| chr6.26778601.26778900 | ENSG00000241549 | hyper |
| chr6.26778901.26779200 | ENSG00000241549 | hyper |
| chr6.26779201.26779500 | ENSG00000241549 | hyper |
| chr6.27258301.27258600 | ENSG00000158553 | hyper |
| chr6.27462901.27463200 | ENSG00000270666 | hyper |
| chr6.27533701.27534000 | ENSG00000219738 | hyper |
| chr6.27534001.27534300 | ENSG00000219738 | hyper |
| chr6.27648601.27648900 | ENSG00000216676 | hyper |
| chr6.27648901.27649200 | ENSG00000216676 | hyper |
| chr6.28740901.28741200 | ENSG00000221191 | hyper |
| chr6.39281101.39281400 | ENSG00000124780 | hyper |
| chr6.58147501.58147800 | ENSG00000272541 | hyper |
| chr5.178978501.178978800 | ENSG00000176783 | hyper |
| chr6.28411201.28411500 | ENSG00000187987 | hyper |
| chr5.170742001.170742300 | ENSG00000164438 | hyper |
| chr5.172665301.172665600 | ENSG00000183072 | hyper |
| chr5.174158701.174159000 | ENSG00000120149 | hyper |
| chr5.174159001.174159300 | ENSG00000120149 | hyper |
| chr5.174159301.174159600 | ENSG00000120149 | hyper |
| chr5.174486901.174487200 | ENSG00000204754 | hyper |
| chr5.177666601.177666900 | ENSG00000050767 | hyper |
| chr5.178368001.178368300 | ENSG00000178187 | hyper |
| chr6.5026501.5026800 | ENSG00000272142 | hyper |
| chr6.6004201.6004500 | ENSG00000124785 | hyper |
| chr6.10382101.10382400 | ENSG00000137203 | hyper |
| chr6.26614201.26614500 | ENSG00000271071 | hyper |
| chr6.26614501.26614800 | ENSG00000271071 | hyper |
| chr6.26614801.26615100 | ENSG00000271071 | hyper |
| chr6.26721301.26721600 | ENSG00000261584 | hyper |
| chr6.26723101.26723400 | ENSG00000261584 | hyper |
| chr6.27279901.27280200 | ENSG00000158553 | hyper |
| chr6.27280201.27280500 | ENSG00000158553 | hyper |
| chr6.27463201.27463500 | ENSG00000270666 | hyper |
| chr6.28303801.28304100 | ENSG00000235109 | hyper |
| chr6.28367101.28367400 | ENSG00000158691 | hyper |
| chr6.28367401.28367700 | ENSG00000158691 | hyper |
| chr6.28414801.28415100 | ENSG00000231162 | hyper |
| chr6.28554901.28555200 | ENSG00000232040 | hyper |
| chr6.28602601.28602900 | ENSG00000271440 | hyper |
| chr6.28753801.28754100 | ENSG00000265764 | hyper |
| chr6.28778101.28778400 | ENSG00000265764 | hyper |
| chr6.32977201.32977500 | ENSG00000263756 | hyper |
| chr6.41341501.41341800 | ENSG00000238867 | hyper |
| chr6.50818801.50819100 | ENSG00000008196 | hyper |
| chr6.56716201.56716500 | ENSG00000151914 | hyper |
| chr6.58147201.58147500 | ENSG00000272541 | hyper |
| chr6.58147801.58148100 | ENSG00000272541 | hyper |
| chr6.58148401.58148700 | ENSG00000272541 | hyper |
| chr6.58148701.58149000 | ENSG00000272541 | hyper |
| chr6.62995501.62995800 | ENSG00000112232 | hyper |
| chr6.74024401.74024700 | ENSG00000135314 | hyper |
| chr6.75794701.75795000 | ENSG00000111799 | hyper |
| chr6.78172201.78172500 | ENSG00000135312 | hyper |
| chr6.78172501.78172800 | ENSG00000135312 | hyper |
| chr6.78173101.78173400 | ENSG00000135312 | hyper |
| chr6.85473001.85473300 | ENSG00000112837 | hyper |
| chr6.99291301.99291600 | ENSG00000184486 | hyper |
| chr6.100056001.100056300 | ENSG00000112238 | hyper |
| chr6.100441801.100442100 | ENSG00000152034 | hyper |
| chr6.100912501.100912800 | ENSG00000112246 | hyper |
| chr6.101847001.101847300 | ENSG00000164418 | hyper |
| chr6.106433701.106434000 | ENSG00000200198 | hyper |
| chr6.108440101.108440400 | ENSG00000081087 | hyper |
| chr6.108488401.108488700 | ENSG00000112333 | hyper |
| chr6.108488701.108489000 | ENSG00000112333 | hyper |
| chr6.108489301.108489600 | ENSG00000112333 | hyper |
| chr6.117086401.117086700 | ENSG00000183807 | hyper |
| chr6.117591301.117591600 | ENSG00000170162 | hyper |
| chr6.133562401.133562700 | ENSG00000112319 | hyper |
| chr6.133562701.133563000 | ENSG00000112319 | hyper |
| chr6.134214001.134214300 | ENSG00000118526 | hyper |
| chr6.137810401.137810700 | ENSG00000177468 | hyper |
| chr7.27260101.27260400 | ENSG00000243766 | hyper |
| chr7.35301001.35301300 | ENSG00000226063 | hyper |
| chr7.1959601.1959900 | ENSG00000002822 | hyper |
| chr7.8474701.8475000 | ENSG00000122584 | hyper |
| chr7.19184701.19185000 | ENSG00000229533 | hyper |
| chr6.137808901.137809200 | ENSG00000177468 | hyper |
| chr6.137816701.137817000 | ENSG00000177468 | hyper |
| chr6.151562401.151562700 | ENSG00000131016 | hyper |
| chr6.159654901.159655200 | ENSG00000164694 | hyper |
| chr6.166074601.166074900 | ENSG00000112541 | hyper |
| chr6.166580401.166580700 | ENSG00000164458 | hyper |
| chr6.166582801.166583100 | ENSG00000164458 | hyper |
| chr6.166583101.166583400 | ENSG00000164458 | hyper |
| chr7.1270801.1271100 | ENSG00000164853 | hyper |
| chr7.8475001.8475300 | ENSG00000122584 | hyper |
| chr7.8475301.8475600 | ENSG00000122584 | hyper |
| chr7.8481301.8481600 | ENSG00000122584 | hyper |
| chr7.8482501.8482800 | ENSG00000122584 | hyper |
| chr7.8482801.8483100 | ENSG00000122584 | hyper |
| chr7.15726601.15726900 | ENSG00000106511 | hyper |
| chr7.19146001.19146300 | ENSG00000122691 | hyper |
| chr7.19146301.19146600 | ENSG00000122691 | hyper |
| chr7.19146901.19147200 | ENSG00000122691 | hyper |
| chr7.19147201.19147500 | ENSG00000122691 | hyper |
| chr7.19152001.19152300 | ENSG00000122691 | hyper |
| chr7.19158001.19158300 | ENSG00000122691 | hyper |
| chr7.19158601.19158900 | ENSG00000236536 | hyper |
| chr7.19184401.19184700 | ENSG00000229533 | hyper |
| chr7.19185001.19185300 | ENSG00000229533 | hyper |
| chr7.22589401.22589700 | ENSG00000105889 | hyper |
| chr7.23507401.23507700 | ENSG00000136231 | hyper |
| chr7.24324301.24324600 | ENSG00000122585 | hyper |
| chr7.24324601.24324900 | ENSG00000122585 | hyper |
| chr7.27192301.27192600 | ENSG00000254369 | hyper |
| chr7.27196501.27196800 | ENSG00000122592 | hyper |
| chr7.27204301.27204600 | ENSG00000078399 | hyper |
| chr7.27204601.27204900 | ENSG00000078399 | hyper |
| chr7.27205201.27205500 | ENSG00000078399 | hyper |
| chr7.27205501.27205800 | ENSG00000078399 | hyper |
| chr7.27205801.27206100 | ENSG00000078399 | hyper |
| chr7.27206101.27206400 | ENSG00000078399 | hyper |
| chr7.27225001.27225300 | ENSG00000240990 | hyper |
| chr7.27244501.27244800 | ENSG00000243766 | hyper |
| chr7.27244801.27245100 | ENSG00000243766 | hyper |
| chr7.27252601.27252900 | ENSG00000243766 | hyper |
| chr7.27284701.27285000 | ENSG00000253405 | hyper |
| chr7.27291301.27291600 | ENSG00000106038 | hyper |
| chr7.27291601.27291900 | ENSG00000106038 | hyper |
| chr7.27291901.27292200 | ENSG00000106038 | hyper |
| chr7.30721201.30721500 | ENSG00000106113 | hyper |
| chr7.31092601.31092900 | ENSG00000078549 | hyper |
| chr7.35293201.35293500 | ENSG00000164532 | hyper |
| chr7.35297401.35297700 | ENSG00000226063 | hyper |
| chr7.35301301.35301600 | ENSG00000226063 | hyper |
| chr7.37955701.37956000 | ENSG00000086289 | hyper |
| chr7.52156201.52156500 | ENSG00000233960 | hyper |
| chr7.54609601.54609900 | ENSG00000170419 | hyper |
| chr7.64349101.64349400 | ENSG00000198039 | hyper |
| chr7.64349401.64349700 | ENSG00000198039 | hyper |
| chr7.71800801.71801100 | ENSG00000183166 | hyper |
| chr7.79083601.79083900 | ENSG00000234456 | hyper |
| chr7.88388101.88388400 | ENSG00000182348 | hyper |
| chr7.93203701.93204000 | ENSG00000004948 | hyper |
| chr7.93519301.93519600 | ENSG00000127928 | hyper |
| chr7.93519601.93519900 | ENSG00000127928 | hyper |
| chr7.94284901.94285200 | ENSG00000127990 | hyper |
| chr7.96647401.96647700 | ENSG00000105880 | hyper |
| chr7.96650701.96651000 | ENSG00000105880 | hyper |
| chr7.96651001.96651300 | ENSG00000105880 | hyper |
| chr7.97362301.97362600 | ENSG00000006128 | hyper |
| chr7.97362601.97362900 | ENSG00000006128 | hyper |
| chr7.97362901.97363200 | ENSG00000006128 | hyper |
| chr7.97363201.97363500 | ENSG00000006128 | hyper |
| chr7.107641801.107642100 | ENSG00000091136 | hyper |
| chr7.107642101.107642400 | ENSG00000091136 | hyper |
| chr7.113722801.113723100 | ENSG00000128573 | hyper |
| chr7.113723101.113723400 | ENSG00000128573 | hyper |
| chr8.99951301.99951600 | ENSG00000104375 | hyper |
| chr8.99951601.99951900 | ENSG00000104375 | hyper |
| chr8.99951901.99952200 | ENSG00000104375 | hyper |
| chr7.123173101.123173400 | ENSG00000164675 | hyper |
| chr8.38008201.38008500 | ENSG00000147465 | hyper |
| chr8.55372201.55372500 | ENSG00000164736 | hyper |
| chr8.60032401.60032700 | ENSG00000167912 | hyper |
| chr8.99960601.99960900 | ENSG00000164920 | hyper |
| chr7.117119401.117119700 | ENSG00000001626 | hyper |
| chr7.121956901.121957200 | ENSG00000081803 | hyper |
| chr7.123172801.123173100 | ENSG00000164675 | hyper |
| chr7.136554001.136554300 | ENSG00000234352 | hyper |
| chr7.136554301.136554600 | ENSG00000234352 | hyper |
| chr7.136554601.136554900 | ENSG00000234352 | hyper |
| chr7.136554901.136555200 | ENSG00000234352 | hyper |
| chr7.137532001.137532300 | ENSG00000157680 | hyper |
| chr7.137532301.137532600 | ENSG00000157680 | hyper |
| chr7.155241901.155242200 | ENSG00000236544 | hyper |
| chr7.155242801.155243100 | ENSG00000236544 | hyper |
| chr7.155243701.155244000 | ENSG00000236544 | hyper |
| chr7.155259301.155259600 | ENSG00000164778 | hyper |
| chr7.155259601.155259900 | ENSG00000164778 | hyper |
| chr7.155301601.155301900 | ENSG00000146910 | hyper |
| chr7.156795601.156795900 | ENSG00000130675 | hyper |
| chr7.156797101.156797400 | ENSG00000130675 | hyper |
| chr7.156797401.156797700 | ENSG00000130675 | hyper |
| chr7.156810901.156811200 | ENSG00000243479 | hyper |
| chr7.156811201.156811500 | ENSG00000243479 | hyper |
| chr7.157482001.157482300 | ENSG00000155093 | hyper |
| chr7.157482301.157482600 | ENSG00000155093 | hyper |
| chr8.4849501.4849800 | ENSG00000183117 | hyper |
| chr8.4849801.4850100 | ENSG00000183117 | hyper |
| chr8.21996601.21996900 | ENSG00000168476 | hyper |
| chr8.23563801.23564100 | ENSG00000180053 | hyper |
| chr8.23564101.23564400 | ENSG00000180053 | hyper |
| chr8.23564401.23564700 | ENSG00000253471 | hyper |
| chr8.24858901.24859200 | ENSG00000253832 | hyper |
| chr8.25905001.25905300 | ENSG00000221818 | hyper |
| chr8.33372001.33372300 | ENSG00000129696 | hyper |
| chr8.33372301.33372600 | ENSG00000129696 | hyper |
| chr8.37655701.37656000 | ENSG00000020181 | hyper |
| chr8.55366201.55366500 | ENSG00000164736 | hyper |
| chr8.55367101.55367400 | ENSG00000164736 | hyper |
| chr8.55367401.55367700 | ENSG00000164736 | hyper |
| chr8.57026101.57026400 | ENSG00000172680 | hyper |
| chr8.65283301.65283600 | ENSG00000253554 | hyper |
| chr8.65290801.65291100 | ENSG00000254377 | hyper |
| chr8.65499601.65499900 | ENSG00000172817 | hyper |
| chr8.67873501.67873800 | ENSG00000261787 | hyper |
| chr8.70981801.70982100 | ENSG00000147596 | hyper |
| chr8.70983901.70984200 | ENSG00000147596 | hyper |
| chr8.70984201.70984500 | ENSG00000147596 | hyper |
| chr8.72470401.72470700 | ENSG00000253379 | hyper |
| chr8.72471001.72471300 | ENSG00000253379 | hyper |
| chr8.72754501.72754800 | ENSG00000235531 | hyper |
| chr8.72754801.72755100 | ENSG00000235531 | hyper |
| chr8.72917101.72917400 | ENSG00000235531 | hyper |
| chr8.72917401.72917700 | ENSG00000235531 | hyper |
| chr8.76316701.76317000 | ENSG00000164749 | hyper |
| chr8.76317001.76317300 | ENSG00000164749 | hyper |
| chr8.85094401.85094700 | ENSG00000184672 | hyper |
| chr8.85094701.85095000 | ENSG00000184672 | hyper |
| chr8.93114001.93114300 | ENSG00000079102 | hyper |
| chr8.97167001.97167300 | ENSG00000156466 | hyper |
| chr8.97170001.97170300 | ENSG00000156466 | hyper |
| chr8.97170301.97170600 | ENSG00000156466 | hyper |
| chr8.97170601.97170900 | ENSG00000156466 | hyper |
| chr8.99952201.99952500 | ENSG00000104375 | hyper |
| chr8.99960301.99960600 | ENSG00000164920 | hyper |
| chr8.99960901.99961200 | ENSG00000164920 | hyper |
| chr8.99961201.99961500 | ENSG00000164920 | hyper |
| chr8.99986101.99986400 | ENSG00000229625 | hyper |
| chr9.970801.971100 | ENSG00000137090 | hyper |
| chr9.1045201.1045500 | ENSG00000173253 | hyper |
| chr9.1045801.1046100 | ENSG00000173253 | hyper |
| chr9.41454901.41455200 | ENSG00000237625 | hyper |
| chr9.79629301.79629600 | ENSG00000204612 | hyper |
| chr8.132053701.132054000 | ENSG00000155897 | hyper |
| chr8.109094701.109095000 | ENSG00000147655 | hyper |
| chr8.114444601.114444900 | ENSG00000164796 | hyper |
| chr8.114444901.114445200 | ENSG00000164796 | hyper |
| chr8.114447001.114447300 | ENSG00000164796 | hyper |
| chr8.132053401.132053700 | ENSG00000155897 | hyper |
| chr8.132054001.132054300 | ENSG00000155897 | hyper |
| chr9.117001.117300 | ENSG00000170122 | hyper |
| chr9.117301.117600 | ENSG00000170122 | hyper |
| chr9.117601.117900 | ENSG00000170122 | hyper |
| chr9.117901.118200 | ENSG00000170122 | hyper |
| chr9.843001.843300 | ENSG00000137090 | hyper |
| chr9.843301.843600 | ENSG00000137090 | hyper |
| chr9.973501.973800 | ENSG00000064218 | hyper |
| chr9.1042501.1042800 | ENSG00000173253 | hyper |
| chr9.1045501.1045800 | ENSG00000173253 | hyper |
| chr9.17907001.17907300 | ENSG00000107295 | hyper |
| chr9.19788301.19788600 | ENSG00000155886 | hyper |
| chr9.19788601.19788900 | ENSG00000155886 | hyper |
| chr9.34809601.34809900 | ENSG00000257198 | hyper |
| chr9.36739501.36739800 | ENSG00000165304 | hyper |
| chr9.36739801.36740100 | ENSG00000165304 | hyper |
| chr9.69201001.69201300 | ENSG00000204793 | hyper |
| chr9.79628701.79629000 | ENSG00000204612 | hyper |
| chr9.79629001.79629300 | ENSG00000204612 | hyper |
| chr9.79630501.79630800 | ENSG00000204612 | hyper |
| chr9.79631401.79631700 | ENSG00000204612 | hyper |
| chr9.79636801.79637100 | ENSG00000204612 | hyper |
| chr9.90114001.90114300 | ENSG00000196730 | hyper |
| chr9.96713401.96713700 | ENSG00000131668 | hyper |
| chr9.96715201.96715500 | ENSG00000131668 | hyper |
| chr9.100610701.100611000 | ENSG00000178919 | hyper |
| chr9.100611301.100611600 | ENSG00000178919 | hyper |
| chr9.133537201.133537500 | ENSG00000130711 | hyper |
| chr10.102996001.102996300 | ENSG00000227128 | hyper |
| chr10.102997201.102997500 | ENSG00000227128 | hyper |
| chr9.124414501.124414800 | ENSG00000136848 | hyper |
| chr9.126775201.126775500 | ENSG00000106689 | hyper |
| chr9.126777301.126777600 | ENSG00000106689 | hyper |
| chr9.127212901.127213200 | ENSG00000180264 | hyper |
| chr9.129380401.129380700 | ENSG00000136944 | hyper |
| chr9.129386101.129386400 | ENSG00000136944 | hyper |
| chr10.8076901.8077200 | ENSG00000197308 | hyper |
| chr10.8077201.8077500 | ENSG00000197308 | hyper |
| chr10.21783301.21783600 | ENSG00000204682 | hyper |
| chr10.22765501.22765800 | ENSG00000077327 | hyper |
| chr10.23462101.23462400 | ENSG00000168267 | hyper |
| chr10.23480401.23480700 | ENSG00000168267 | hyper |
| chr10.28035001.28035300 | ENSG00000230500 | hyper |
| chr10.44879101.44879400 | ENSG00000107562 | hyper |
| chr10.50605501.50605800 | ENSG00000165606 | hyper |
| chr10.63212401.63212700 | ENSG00000196932 | hyper |
| chr10.71337601.71337900 | ENSG00000236154 | hyper |
| chr10.94828201.94828500 | ENSG00000187553 | hyper |
| chr10.94833901.94834200 | ENSG00000095596 | hyper |
| chr10.102894901.102895200 | ENSG00000107807 | hyper |
| chr10.102996301.102996600 | ENSG00000227128 | hyper |
| chr10.106400401.106400700 | ENSG00000156395 | hyper |
| chr10.110671801.110672100 | ENSG00000222436 | hyper |
| chr10.118031101.118031400 | ENSG00000151892 | hyper |
| chr10.118031401.118031700 | ENSG00000151892 | hyper |
| chr10.118033801.118034100 | ENSG00000151892 | hyper |
| chr10.118891501.118891800 | ENSG00000148704 | hyper |
| chr10.118892401.118892700 | ENSG00000148704 | hyper |
| chr10.119301301.119301600 | ENSG00000229847 | hyper |
| chr10.119304901.119305200 | ENSG00000170370 | hyper |
| chr10.119305201.119305500 | ENSG00000170370 | hyper |
| chr10.119494201.119494500 | ENSG00000234952 | hyper |
| chr10.119494501.119494800 | ENSG00000234952 | hyper |
| chr11.18813901.18814200 | ENSG00000110786 | hyper |
| chr11.31826401.31826700 | ENSG00000007372 | hyper |
| chr11.69832501.69832800 | ENSG00000202070 | hyper |
| chr10.122709601.122709900 | ENSG00000227307 | hyper |
| chr10.124896601.124896900 | ENSG00000188620 | hyper |
| chr10.124905601.124905900 | ENSG00000188816 | hyper |
| chr10.124908901.124909200 | ENSG00000188816 | hyper |
| chr10.131761201.131761500 | ENSG00000108001 | hyper |
| chr10.131767801.131768100 | ENSG00000108001 | hyper |
| chr11.7041601.7041900 | ENSG00000158077 | hyper |
| chr11.14995501.14995800 | ENSG00000175868 | hyper |
| chr11.22363201.22363500 | ENSG00000091664 | hyper |
| chr11.31825801.31826100 | ENSG00000007372 | hyper |
| chr11.31826101.31826400 | ENSG00000007372 | hyper |
| chr11.31827001.31827300 | ENSG00000007372 | hyper |
| chr11.32454601.32454900 | ENSG00000184937 | hyper |
| chr11.32455801.32456100 | ENSG00000184937 | hyper |
| chr11.32459401.32459700 | ENSG00000183242 | hyper |
| chr11.32459701.32460000 | ENSG00000183242 | hyper |
| chr11.35641801.35642100 | ENSG00000179431 | hyper |
| chr11.43602901.43603200 | ENSG00000149084 | hyper |
| chr11.62693701.62694000 | ENSG00000168539 | hyper |
| chr11.66188701.66189000 | ENSG00000174576 | hyper |
| chr11.69451501.69451800 | ENSG00000110092 | hyper |
| chr11.69451801.69452100 | ENSG00000110092 | hyper |
| chr11.69452101.69452400 | ENSG00000110092 | hyper |
| chr11.69452401.69452700 | ENSG00000110092 | hyper |
| chr11.69517501.69517800 | ENSG00000162344 | hyper |
| chr11.69517801.69518100 | ENSG00000162344 | hyper |
| chr11.69831901.69832200 | ENSG00000202070 | hyper |
| chr11.69832201.69832500 | ENSG00000202070 | hyper |
| chr11.70211401.70211700 | ENSG00000131626 | hyper |
| chr11.91958401.91958700 | ENSG00000242248 | hyper |
| chr11.100999201.100999500 | ENSG00000082175 | hyper |
| chr11.100999501.100999800 | ENSG00000082175 | hyper |
| chr11.101453101.101453400 | ENSG00000137672 | hyper |
| chr11.101453401.101453700 | ENSG00000137672 | hyper |
| chr11.122848201.122848500 | ENSG00000188909 | hyper |
| chr11.123066601.123066900 | ENSG00000254710 | hyper |
| chr12.54424501.54424800 | ENSG00000273049 | hyper |
| chr12.106974901.106975200 | ENSG00000257545 | hyper |
| chr12.115173301.115173600 | ENSG00000257817 | hyper |
| chr12.128752501.128752800 | ENSG00000181234 | hyper |
| chr12.6184501.6184800 | ENSG00000110799 | hyper |
| chr12.14134201.14134500 | ENSG00000273079 | hyper |
| chr12.16500601.16500900 | ENSG00000008394 | hyper |
| chr12.22093801.22094100 | ENSG00000069431 | hyper |
| chr12.22094701.22095000 | ENSG00000069431 | hyper |
| chr12.25056301.25056600 | ENSG00000060982 | hyper |
| chr12.30323101.30323400 | ENSG00000257262 | hyper |
| chr12.43944901.43945200 | ENSG00000173157 | hyper |
| chr12.48397201.48397500 | ENSG00000139219 | hyper |
| chr12.54321301.54321600 | ENSG00000249641 | hyper |
| chr12.54329701.54330000 | ENSG00000249641 | hyper |
| chr12.54338701.54339000 | ENSG00000123364 | hyper |
| chr12.54339001.54339300 | ENSG00000123364 | hyper |
| chr12.54339301.54339600 | ENSG00000123364 | hyper |
| chr12.54345901.54346200 | ENSG00000123407 | hyper |
| chr12.54354601.54354900 | ENSG00000228630 | hyper |
| chr12.54408301.54408600 | ENSG00000273049 | hyper |
| chr12.54408601.54408900 | ENSG00000273049 | hyper |
| chr12.54423301.54423600 | ENSG00000273049 | hyper |
| chr12.54424801.54425100 | ENSG00000273049 | hyper |
| chr12.54441001.54441300 | ENSG00000198353 | hyper |
| chr12.58021801.58022100 | ENSG00000135454 | hyper |
| chr12.81471601.81471900 | ENSG00000111058 | hyper |
| chr12.85673101.85673400 | ENSG00000180318 | hyper |
| chr12.85673401.85673700 | ENSG00000180318 | hyper |
| chr12.85674301.85674600 | ENSG00000180318 | hyper |
| chr12.95941801.95942100 | ENSG00000136014 | hyper |
| chr12.103344301.103344600 | ENSG00000171759 | hyper |
| chr12.106979401.106979700 | ENSG00000257545 | hyper |
| chr12.114838201.114838500 | ENSG00000089225 | hyper |
| chr12.114845701.114846000 | ENSG00000089225 | hyper |
| chr12.114846301.114846600 | ENSG00000255399 | hyper |
| chr12.114846601.114846900 | ENSG00000255399 | hyper |
| chr12.114847501.114847800 | ENSG00000255399 | hyper |
| chr12.114878101.114878400 | ENSG00000255399 | hyper |
| chr12.114878401.114878700 | ENSG00000255399 | hyper |
| chr12.114878701.114879000 | ENSG00000255399 | hyper |
| chr12.115107301.115107600 | ENSG00000135111 | hyper |
| chr12.115109401.115109700 | ENSG00000135111 | hyper |
| chr12.115173601.115173900 | ENSG00000257817 | hyper |
| chr12.128752201.128752500 | ENSG00000181234 | hyper |
| chr12.133484701.133485000 | ENSG00000072609 | hyper |
| chr12.133485001.133485300 | ENSG00000072609 | hyper |
| chr12.133485301.133485600 | ENSG00000072609 | hyper |
| chr13.58203601.58203900 | ENSG00000118946 | hyper |
| chr13.112716601.112716900 | ENSG00000182968 | hyper |
| chr13.78493201.78493500 | ENSG00000136160 | hyper |
| chr14.38724601.38724900 | ENSG00000176435 | hyper |
| chr14.38724901.38725200 | ENSG00000176435 | hyper |
| chr14.42077401.42077700 | ENSG00000165379 | hyper |
| chr13.23500201.23500500 | ENSG00000262198 | hyper |
| chr13.25320301.25320600 | ENSG00000231417 | hyper |
| chr13.25320601.25320900 | ENSG00000231417 | hyper |
| chr13.28492201.28492500 | ENSG00000247381 | hyper |
| chr13.28552801.28553100 | ENSG00000183463 | hyper |
| chr13.28674001.28674300 | ENSG00000122025 | hyper |
| chr13.58203901.58204200 | ENSG00000118946 | hyper |
| chr13.58206001.58206300 | ENSG00000118946 | hyper |
| chr13.78492901.78493200 | ENSG00000136160 | hyper |
| chr13.79170601.79170900 | ENSG00000234377 | hyper |
| chr13.95354701.95355000 | ENSG00000238230 | hyper |
| chr13.100608601.100608900 | ENSG00000139800 | hyper |
| chr13.100620301.100620600 | ENSG00000139800 | hyper |
| chr13.100641301.100641600 | ENSG00000043355 | hyper |
| chr13.100641601.100641900 | ENSG00000043355 | hyper |
| chr13.100641901.100642200 | ENSG00000043355 | hyper |
| chr13.108520201.108520500 | ENSG00000204442 | hyper |
| chr13.108520501.108520800 | ENSG00000204442 | hyper |
| chr13.108520801.108521100 | ENSG00000204442 | hyper |
| chr13.109147501.109147800 | ENSG00000232087 | hyper |
| chr13.109148401.109148700 | ENSG00000232087 | hyper |
| chr13.109148701.109149000 | ENSG00000232087 | hyper |
| chr13.112708501.112708800 | ENSG00000200072 | hyper |
| chr13.112712401.112712700 | ENSG00000200072 | hyper |
| chr14.29234701.29235000 | ENSG00000176165 | hyper |
| chr14.29235001.29235300 | ENSG00000176165 | hyper |
| chr14.29254501.29254800 | ENSG00000186960 | hyper |
| chr14.36979801.36980100 | ENSG00000257520 | hyper |
| chr14.36982201.36982500 | ENSG00000257520 | hyper |
| chr14.36982501.36982800 | ENSG00000257520 | hyper |
| chr14.36983401.36983700 | ENSG00000257520 | hyper |
| chr14.36983701.36984000 | ENSG00000257520 | hyper |
| chr14.36991801.36992100 | ENSG00000253563 | hyper |
| chr14.37116301.37116600 | ENSG00000258661 | hyper |
| chr14.37123501.37123800 | ENSG00000258661 | hyper |
| chr14.37128601.37128900 | ENSG00000198807 | hyper |
| chr14.38724301.38724600 | ENSG00000176435 | hyper |
| chr14.42074401.42074700 | ENSG00000258636 | hyper |
| chr14.52781701.52782000 | ENSG00000125384 | hyper |
| chr15.45996601.45996900 | ENSG00000259200 | hyper |
| chr15.75251401.75251700 | ENSG00000198794 | hyper |
| chr15.79383001.79383300 | ENSG00000058335 | hyper |
| chr14.52534801.52535100 | ENSG00000087303 | hyper |
| chr14.52535101.52535400 | ENSG00000087303 | hyper |
| chr14.52535401.52535700 | ENSG00000087303 | hyper |
| chr14.52536001.52536300 | ENSG00000087303 | hyper |
| chr14.52536301.52536600 | ENSG00000087303 | hyper |
| chr14.52734901.52735200 | ENSG00000168229 | hyper |
| chr14.52735501.52735800 | ENSG00000168229 | hyper |
| chr14.57261901.57262200 | ENSG00000270163 | hyper |
| chr14.57274801.57275100 | ENSG00000165588 | hyper |
| chr14.57275101.57275400 | ENSG00000165588 | hyper |
| chr14.57275401.57275700 | ENSG00000165588 | hyper |
| chr14.57276301.57276600 | ENSG00000165588 | hyper |
| chr14.57278701.57279000 | ENSG00000248550 | hyper |
| chr14.57279001.57279300 | ENSG00000248550 | hyper |
| chr14.60386401.60386700 | ENSG00000261120 | hyper |
| chr14.60975301.60975600 | ENSG00000179008 | hyper |
| chr14.60976201.60976500 | ENSG00000179008 | hyper |
| chr14.60976501.60976800 | ENSG00000179008 | hyper |
| chr14.61104601.61104900 | ENSG00000258952 | hyper |
| chr14.61109101.61109400 | ENSG00000258952 | hyper |
| chr14.61109701.61110000 | ENSG00000126778 | hyper |
| chr14.61110001.61110300 | ENSG00000126778 | hyper |
| chr14.61110601.61110900 | ENSG00000126778 | hyper |
| chr14.95234701.95235000 | ENSG00000133937 | hyper |
| chr14.95237701.95238000 | ENSG00000133937 | hyper |
| chr14.95238001.95238300 | ENSG00000133937 | hyper |
| chr14.99713101.99713400 | ENSG00000127152 | hyper |
| chr15.53075701.53076000 | ENSG00000169856 | hyper |
| chr15.53076601.53076900 | ENSG00000169856 | hyper |
| chr15.53080801.53081100 | ENSG00000169856 | hyper |
| chr15.76632001.76632300 | ENSG00000159556 | hyper |
| chr15.76633201.76633500 | ENSG00000159556 | hyper |
| chr15.79382701.79383000 | ENSG00000058335 | hyper |
| chr15.81410701.81411000 | ENSG00000156206 | hyper |
| chr15.88800601.88800900 | ENSG00000260305 | hyper |
| chr15.89903401.89903700 | ENSG00000255571 | hyper |
| chr15.89949301.89949600 | ENSG00000255571 | hyper |
| chr15.89949601.89949900 | ENSG00000255571 | hyper |
| chr15.89949901.89950200 | ENSG00000255571 | hyper |
| chr15.89952001.89952300 | ENSG00000255571 | hyper |
| chr16.51189001.51189300 | ENSG00000103449 | hyper |
| chr16.54324001.54324300 | ENSG00000177508 | hyper |
| chr17.46796401.46796700 | ENSG00000159182 | hyper |
| chr17.46832101.46832400 | ENSG00000170703 | hyper |
| chr17.48042901.48043200 | ENSG00000199492 | hyper |
| chr15.95388301.95388600 | ENSG00000260521 | hyper |
| chr15.95388601.95388900 | ENSG00000260521 | hyper |
| chr16.51184801.51185100 | ENSG00000103449 | hyper |
| chr16.89268601.89268900 | ENSG00000259803 | hyper |
| chr17.5974201.5974500 | ENSG00000179314 | hyper |
| chr15.96911401.96911700 | ENSG00000259275 | hyper |
| chr15.96959401.96959700 | ENSG00000259542 | hyper |
| chr16.3220501.3220800 | ENSG00000262521 | hyper |
| chr16.12994501.12994800 | ENSG00000237515 | hyper |
| chr16.12994801.12995100 | ENSG00000237515 | hyper |
| chr16.29086201.29086500 | ENSG00000260908 | hyper |
| chr16.49314601.49314900 | ENSG00000102924 | hyper |
| chr16.49314901.49315200 | ENSG00000102924 | hyper |
| chr16.51190201.51190500 | ENSG00000103449 | hyper |
| chr16.54318001.54318300 | ENSG00000177508 | hyper |
| chr16.54322201.54322500 | ENSG00000177508 | hyper |
| chr16.54970501.54970800 | ENSG00000259711 | hyper |
| chr16.54971401.54971700 | ENSG00000259711 | hyper |
| chr16.54971701.54972000 | ENSG00000259711 | hyper |
| chr16.54972301.54972600 | ENSG00000259711 | hyper |
| chr16.55362901.55363200 | ENSG00000259283 | hyper |
| chr16.55363201.55363500 | ENSG00000259283 | hyper |
| chr16.55364701.55365000 | ENSG00000259283 | hyper |
| chr16.55365001.55365300 | ENSG00000259283 | hyper |
| chr16.55365301.55365600 | ENSG00000259283 | hyper |
| chr16.56672101.56672400 | ENSG00000205362 | hyper |
| chr16.86529901.86530200 | ENSG00000268388 | hyper |
| chr17.7976101.7976400 | ENSG00000179477 | hyper |
| chr17.8868601.8868900 | ENSG00000141506 | hyper |
| chr17.8907601.8907900 | ENSG00000065320 | hyper |
| chr17.26554801.26555100 | ENSG00000237575 | hyper |
| chr17.27942301.27942600 | ENSG00000264031 | hyper |
| chr17.35285401.35285700 | ENSG00000255509 | hyper |
| chr17.36103201.36103500 | ENSG00000108753 | hyper |
| chr17.36103501.36103800 | ENSG00000108753 | hyper |
| chr17.36103801.36104100 | ENSG00000108753 | hyper |
| chr17.36104101.36104400 | ENSG00000108753 | hyper |
| chr17.37321501.37321800 | ENSG00000141748 | hyper |
| chr17.43974601.43974900 | ENSG00000186868 | hyper |
| chr17.46673701.46674000 | ENSG00000120093 | hyper |
| chr17.46796101.46796400 | ENSG00000159182 | hyper |
| chr17.46811701.46812000 | ENSG00000242407 | hyper |
| chr17.46824901.46825200 | ENSG00000242407 | hyper |
| chr17.47301901.47302200 | ENSG00000173868 | hyper |
| chr17.48042301.48042600 | ENSG00000199492 | hyper |
| chr17.48042601.48042900 | ENSG00000199492 | hyper |
| chr18.19746901.19747200 | ENSG00000266010 | hyper |
| chr19.2488501.2488800 | ENSG00000099860 | hyper |
| chr17.70113301.70113600 | ENSG00000234899 | hyper |
| chr18.907201.907500 | ENSG00000265671 | hyper |
| chr18.22929301.22929600 | ENSG00000198795 | hyper |
| chr18.44335801.44336100 | ENSG00000101638 | hyper |
| chr18.49868401.49868700 | ENSG00000187323 | hyper |
| chr19.20606101.20606400 | ENSG00000231205 | hyper |
| chr19.20606401.20606700 | ENSG00000231205 | hyper |
| chr19.37287901.37288200 | ENSG00000267254 | hyper |
| chr19.37288201.37288500 | ENSG00000267254 | hyper |
| chr19.38042401.38042700 | ENSG00000267470 | hyper |
| chr17.59534701.59535000 | ENSG00000121075 | hyper |
| chr17.62075101.62075400 | ENSG00000264954 | hyper |
| chr17.70112401.70112700 | ENSG00000234899 | hyper |
| chr17.70112701.70113000 | ENSG00000234899 | hyper |
| chr17.75370201.75370500 | ENSG00000184640 | hyper |
| chr18.905101.905400 | ENSG00000265671 | hyper |
| chr18.906601.906900 | ENSG00000265671 | hyper |
| chr18.906901.907200 | ENSG00000265671 | hyper |
| chr18.10032601.10032900 | ENSG00000263630 | hyper |
| chr18.12307501.12307800 | ENSG00000176014 | hyper |
| chr18.19745701.19746000 | ENSG00000266010 | hyper |
| chr18.19747501.19747800 | ENSG00000266010 | hyper |
| chr18.22929001.22929300 | ENSG00000198795 | hyper |
| chr18.31803001.31803300 | ENSG00000101746 | hyper |
| chr18.44790001.44790300 | ENSG00000215474 | hyper |
| chr18.55105801.55106100 | ENSG00000119547 | hyper |
| chr18.63418501.63418800 | ENSG00000081138 | hyper |
| chr18.63418801.63419100 | ENSG00000081138 | hyper |
| chr18.67068601.67068900 | ENSG00000206052 | hyper |
| chr18.67068901.67069200 | ENSG00000206052 | hyper |
| chr18.74961601.74961900 | ENSG00000166573 | hyper |
| chr18.76734601.76734900 | ENSG00000263146 | hyper |
| chr19.9608701.9609000 | ENSG00000198028 | hyper |
| chr19.12306001.12306300 | ENSG00000234773 | hyper |
| chr19.16479901.16480200 | ENSG00000127527 | hyper |
| chr19.20844001.20844300 | ENSG00000269110 | hyper |
| chr19.21182701.21183000 | ENSG00000268326 | hyper |
| chr19.22715101.22715400 | ENSG00000197360 | hyper |
| chr19.38182801.38183100 | ENSG00000120784 | hyper |
| chr19.38183101.38183400 | ENSG00000120784 | hyper |
| chr20.55500001.55500300 | ENSG00000251772 | hyper |
| chr19.46907401.46907700 | ENSG00000169515 | hyper |
| chr19.46993201.46993500 | ENSG00000230510 | hyper |
| chr19.48918001.48918300 | ENSG00000105464 | hyper |
| chr19.48918301.48918600 | ENSG00000105464 | hyper |
| chr19.58238401.58238700 | ENSG00000269026 | hyper |
| chr21.38082901.38083200 | ENSG00000159263 | hyper |
| chr21.46360201.46360500 | ENSG00000160256 | hyper |
| chr19.44952301.44952600 | ENSG00000267188 | hyper |
| chr19.44952601.44952900 | ENSG00000267188 | hyper |
| chr19.46929901.46930200 | ENSG00000169515 | hyper |
| chr19.52839301.52839600 | ENSG00000269535 | hyper |
| chr19.52839601.52839900 | ENSG00000269535 | hyper |
| chr19.52873201.52873500 | ENSG00000221923 | hyper |
| chr19.53073301.53073600 | ENSG00000167562 | hyper |
| chr19.54401701.54402000 | ENSG00000126583 | hyper |
| chr19.56879701.56880000 | ENSG00000131848 | hyper |
| chr19.56904901.56905200 | ENSG00000018869 | hyper |
| chr19.56989201.56989500 | ENSG00000198046 | hyper |
| chr19.56989501.56989800 | ENSG00000166770 | hyper |
| chr19.58095001.58095300 | ENSG00000171649 | hyper |
| chr19.58220401.58220700 | ENSG00000204519 | hyper |
| chr19.58238701.58239000 | ENSG00000269026 | hyper |
| chr19.58400101.58400400 | ENSG00000204514 | hyper |
| chr19.58520701.58521000 | ENSG00000176593 | hyper |
| chr19.58873201.58873500 | ENSG00000268230 | hyper |
| chr19.58951201.58951500 | ENSG00000131849 | hyper |
| chr19.58951501.58951800 | ENSG00000131849 | hyper |
| chr20.291001.291300 | ENSG00000225377 | hyper |
| chr20.865801.866100 | ENSG00000101280 | hyper |
| chr20.5296201.5296500 | ENSG00000101292 | hyper |
| chr20.5296501.5296800 | ENSG00000101292 | hyper |
| chr20.5296801.5297100 | ENSG00000101292 | hyper |
| chr20.5297101.5297400 | ENSG00000101292 | hyper |
| chr20.9489301.9489600 | ENSG00000225988 | hyper |
| chr20.9495301.9495600 | ENSG00000225988 | hyper |
| chr20.10198501.10198800 | ENSG00000227906 | hyper |
| chr20.21501901.21502200 | ENSG00000125820 | hyper |
| chr20.21681901.21682200 | ENSG00000125813 | hyper |
| chr20.21687301.21687600 | ENSG00000125813 | hyper |
| chr20.21694201.21694500 | ENSG00000125813 | hyper |
| chr20.21694501.21694800 | ENSG00000125813 | hyper |
| chr20.21694801.21695100 | ENSG00000125813 | hyper |
| chr20.22548901.22549200 | ENSG00000259974 | hyper |
| chr20.22549201.22549500 | ENSG00000259974 | hyper |
| chr20.22558201.22558500 | ENSG00000259974 | hyper |
| chr20.22563601.22563900 | ENSG00000125798 | hyper |
| chr20.37356301.37356600 | ENSG00000101438 | hyper |
| chr20.37357501.37357800 | ENSG00000101438 | hyper |
| chr20.45087601.45087900 | ENSG00000215452 | hyper |
| chr20.45087901.45088200 | ENSG00000215452 | hyper |
| chr20.55500901.55501200 | ENSG00000251772 | hyper |
| chr21.22369801.22370100 | ENSG00000154654 | hyper |
| chr21.34398301.34398600 | ENSG00000227757 | hyper |
| chr21.34398601.34398900 | ENSG00000227757 | hyper |
| chr21.34443301.34443600 | ENSG00000227757 | hyper |
| chr21.34444201.34444500 | ENSG00000184221 | hyper |
| chr21.38066701.38067000 | ENSG00000224269 | hyper |
| chr21.38069401.38069700 | ENSG00000224269 | hyper |
| chr21.38069701.38070000 | ENSG00000224269 | hyper |
| chr21.38077201.38077500 | ENSG00000159263 | hyper |
| chr21.38077501.38077800 | ENSG00000159263 | hyper |
| chr22.48963001.48963300 | ENSG00000219438 | hyper |
| chr22.50629801.50630100 | ENSG00000170638 | hyper |
| chr22.51042001.51042300 | ENSG00000008735 | hyper |
| chr1.147736201.147736500 | ENSG00000199879 | hypo |
| chr1.27319201.27319500 | ENSG00000253368 | hypo |
| chr1.50489401.50489700 | ENSG00000186094 | hypo |
| chr1.11097601.11097900 | ENSG00000009724 | hypo |
| chr2.26593501.26593800 | ENSG00000138018 | hypo |
| chr2.39004801.39005100 | ENSG00000152147 | hypo |
| chr1.155826901.155827200 | ENSG00000116580 | hypo |
| chr2.44314201.44314500 | ENSG00000219391 | hypo |
| chr2.96810301.96810600 | ENSG00000158050 | hypo |
| chr2.96970501.96970800 | ENSG00000144028 | hypo |
| chr2.239685301.239685600 | ENSG00000226992 | hypo |
| chr4.181317301.181317600 | ENSG00000251025 | hypo |
| chr4.159644701.159645000 | ENSG00000171497 | hypo |
| chr5.391201.391500 | ENSG00000063438 | hypo |
| chr5.5886901.5887200 | ENSG00000261037 | hypo |
| chr5.34306501.34306800 | ENSG00000215158 | hypo |
| chr5.125937001.125937300 | ENSG00000164902 | hypo |
| chr6.35181601.35181900 | ENSG00000146197 | hypo |
| chr6.114180901.114181200 | ENSG00000155130 | hypo |
| chr5.170745301.170745600 | ENSG00000164438 | hypo |
| chr6.37070101.37070400 | ENSG00000216412 | hypo |
| chr7.6387901.6388200 | ENSG00000178397 | hypo |
| chr7.5553601.5553900 | ENSG00000155034 | hypo |
| chr7.57484501.57484800 | ENSG00000270957 | hypo |
| chr7.130275301.130275600 | ENSG00000239021 | hypo |
| chr8.17770201.17770500 | ENSG00000104760 | hypo |
| chr9.1009201.1009500 | ENSG00000228783 | hypo |
| chr9.132388201.132388500 | ENSG00000148335 | hypo |
| chr9.136890301.136890600 | ENSG00000235106 | hypo |
| chr10.71905201.71905500 | ENSG00000156521 | hypo |
| chr10.1584301.1584600 | ENSG00000185736 | hypo |
| chr11.1404601.1404900 | ENSG00000174672 | hypo |
| chr12.52317301.52317600 | ENSG00000139567 | hypo |
| chr12.122459101.122459400 | ENSG00000110987 | hypo |
| chr12.122687701.122688000 | ENSG00000158113 | hypo |
| chr12.130527001.130527300 | ENSG00000261650 | hypo |
| chr12.133050001.133050300 | ENSG00000269676 | hypo |
| chr14.50540401.50540700 | ENSG00000273065 | hypo |
| chr14.103995001.103995300 | ENSG00000260285 | hypo |
| chr17.17739301.17739600 | ENSG00000072310 | hypo |
| chr16.67562401.67562700 | ENSG00000039523 | hypo |
| chr16.84545101.84545400 | ENSG00000140950 | hypo |
| chr16.89939401.89939700 | ENSG00000141002 | hypo |
| chr17.46184401.46184700 | ENSG00000002919 | hypo |
| chr16.3209101.3209400 | ENSG00000261889 | hypo |
| chr19.17457001.17457300 | ENSG00000130299 | hypo |
| chr19.30363901.30364200 | ENSG00000267433 | hypo |
| chr17.75468301.75468600 | ENSG00000184640 | hypo |
| chr17.81082801.81083100 | ENSG00000262898 | hypo |
| chr19.8408101.8408400 | ENSG00000186994 | hypo |
| chr19.14332201.14332500 | ENSG00000240803 | hypo |
| chr20.60717001.60717300 | ENSG00000101182 | hypo |
| chr21.9438301.9438600 | ENSG00000238411 | hypo |
| chr19.45004801.45005100 | ENSG00000167384 | hypo |
| chr19.50880001.50880300 | ENSG00000131408 | hypo |
| chr20.45439801.45440100 | ENSG00000266136 | hypo |

Genomic Mutation Profile

The present disclosure provides methods, systems, and kits for producing a mutation profile of a subject that has a disease/condition or is suspected of having such disease/condition, wherein the methylation profile may be used to determine whether the subject has the disease/condition or is at risk of having the disease/condition. The samples disclosed herein are subjected to library preparation and next generation deep sequencing (e.g., CAPP-Seq). A plurality of sequencing reads is generated and analyzed. In some embodiments, deep sequencing may be configured to maximize identifying genomic mutations associated with the disease/condition. For example, not meant to be limiting, for head and neck squamous cell carcinoma (HNSCC), a panel of canonical HNSCC driver genes may be included in the selector for CAPP-seq. Further, for lung cancer, a panel of lung cancer drive genes may be included in the selector for CAPP-seq. Moreover, for pancreatic cancer, a panel of pancreatic cancer drive genes may be included in the selector for CAPP-seq. In some embodiments, including genes without known driver effects in a particular cancer type in the selector for CAPP-seq may increase the sensitivity of ctDNA detection.

In some embodiments, the relative measure of ctDNA abundance is calculate from the mean mutant allele fractions (MAFs). In some embodiments, the mean MAF of mutations identified a subject and comprised in his/her mutation profile ranges from at least about 0.01% to at least about 10%. The ctDNA fraction of a sample disclosed herein is about at least 0.01%, 0.02%, 0.03%, 0.04%, 0.05%, 0.06%, 0.07%, 0.08%, 0.09%, 0.1%, 0.15%, 0.2%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, or any percentage in between.

In some embodiments, the generated mutation profile of a subject does not include mutation variants derived from cell-free nucleic acid molecules derived from PBLs. In some embodiments, the mutation profile comprises genetic polymorphisms, such as missense variant, a nonsense variant, a deletion variant, an insertion variant, a duplication variant, an inversion variant, a frameshift variant, or a repeat expansion variant. In some embodiments, the mutation profile may comprise mutation variant derived from a fraction of cell-free nucleic acid molecules of a specific size range.

Fragment Length Profile

In some embodiment, the length of ctDNA fragments is shorter than cell-free nucleic acid molecules derived from a healthy subject. In some embodiments, the length of ctDNA comprising at least one mutation is shorter than the length of cell free nucleic acid molecule containing a corresponding reference allele. In some embodiments, a length of a ctDNA fragment containing at least one DMR is shorter than a cell-free nucleic acid molecule fragment containing the corresponding genomic region.

In some embodiments, the sequencing does not utilize bisulfite sequence because it causes degradation of ctDNA fragments and prevents the preservation of the length distribution of ctDNAs. In some embodiments, the fragment length of ctDNA is at least from 60 to 500 bp, 80 to 300 bp, 90 to 250 bp, 80 to 170 bp, or 100 to 150 bp. In some embodiments, the present disclosure provides an enrichment of the cell free nucleic acid samples based on selecting cell free molecules of a certain size. In some embodiments, the multimodal analysis comprises utilizing the mutation profile described herein and the fragment length profile by selectively including a plurality of nucleic acid molecules in the mutation profile based on their fragment length. In some embodiments, the multimodal analysis comprises utilizing the methylation profile described herein and the fragment length profile by selectively including a plurality of nucleic acid molecules in the methylation profile based on their fragment length. In some embodiments, the multimodal analysis comprises utilizing the mutation profile, methylation profile, and the fragment length profile together by selectively including a plurality of nucleic acid molecules in the mutation profile based on their fragment length and by selectively including a plurality of nucleic acid molecules in the methylation profile based on their fragment length respectively.

Methods and Systems for Detecting Cancer, Determining Tissue of Origin for Tumor, and Providing Prognosis

The present disclosure provides methods and systems for determining whether a subject has or is at risk of having a disease, wherein the methods and systems comprises subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and processing said at least one profile to determine whether said subject has or is at risk of said disease at a sensitivity of at least 80% or at a specificity of at least about 90%, wherein said cell-free nucleic acid sample comprises less than 30 ng/ml of said plurality of nucleic acid molecules. In some embodiments, the sensitivity is at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the specificity is at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

In some embodiments, the methods and systems comprises subjecting a plurality of nucleic acid molecules derived from a cell-free nucleic acid sample obtained from said subject to sequencing to generate at least two profiles of (i) a methylation profile, (ii) a mutation profile, and (iii) a fragment length profile. The methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the sensitivity when using two profiles is increased by at least about 0.5%, 1%, 2%, 3%, 4%, 5%, 6%, 7%, 8%, 9%, 10%, or percentage in between any of the numbers compared to the sensitivity when using one profile. In some embodiments, the sensitivity when using three profiles is increased by at least about 0.5%, 1%, 2%, 3%, 4%, 5%, 6%, 7%, 8%, 9%, 10%, or percentage in between any of the numbers compared to the sensitivity when using two profile.

Further, the methods provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers. In some embodiments, the specificity when using two profiles is increased by at least about 0.5%, 1%, 2%, 3%, 4%, 5%, 6%, 7%, 8%, 9%, 10%, or percentage in between any of the numbers compared to the specificity when using one profile. In some embodiments, the specificity when using three profiles is increased by at least about 0.5%, 1%, 2%, 3%, 4%, 5%, 6%, 7%, 8%, 9%, 10%, or percentage in between any of the numbers compared to the specificity when using two profile.

The present disclosure provides methods and systems for processing a cell-free nucleic acid sample of a subject to determine whether said subject has or is at risk of having a disease, the methods and systems comprise providing said cell-free nucleic acid sample comprising a plurality of nucleic acid molecules; subjecting said plurality of nucleic acid molecules or derivatives thereof to sequencing to generate a plurality of sequencing reads; computer processing said plurality of sequencing reads to identify, for said plurality of nucleic acid molecules, (i) a methylation profile, (ii) a mutation profile, and (iii) a fragment length profile; and using at least said methylation profile, said mutation profile and said fragment length profile to determine whether said subject has or is at risk of having said disease. In some embodiments, the methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. The methods provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

The present disclosure provides methods and systems for determining a tissue origin of a tumor, comprising identifying a plurality of Differentially Methylated Regions (DMRs), wherein the plurality of DMRs is specific for a particular cancer (e.g., breast cancer, colon cancer, prostate cancer, HSNCC) and derived from a fraction of cell-free nucleic acid molecules. In some embodiments, the fraction of the cell-free nucleic acid molecules is derived from ctDNA. In some embodiments, the methods provides a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. The methods provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

The present disclosure describes methods and systems for providing a prognosis to a subject after receiving a treatment for a disease/condition. For example, the treatment comprises a surgical removal of a tumor, a chemotherapy designed for a specific type of cancer, a radio therapy, or an immune therapy (e.g., TCR, CAR, etc.). In some embodiments, the methods or systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and monitoring or detecting minimal residual disease (MRD) based at least based on the at least one profile.

The present disclosure provides methods and systems for determining whether a subject has a disease/condition by assaying a cell-free nucleic acid molecule from at least a portion of a sample from said subject; detecting a methylation level of at least a portion of said cell-free nucleic acid molecule comprised in a differentially methylated region (DMR) listed in Table 5; and comparing, using at least one computer processor, said methylation level detected in (b) to a methylation level of corresponding portion(s) of said cell-free nucleic acid molecules comprised in said DMR listed in Table 5. In some embodiments, the methylation level of at least about six or more, ten or more, fifteen or more, twenty or more, thirty or more, forty or more, fifty or more, sixty or more, seventy or more, eighty or more, ninety or more, or one hundred or more, two hundred or more, three hundred or more, four hundred or more, five hundred or more, six hundred or more, or seven hundred or more DMRs listed in Table 5 is measured and compared to the methylation level of the corresponding DMRs in a healthy subject as discussed herein.

Once a subject is accurately diagnosed and receives a treatment to treat the cancer, such as surgical removal, chemotherapy, radio therapy, etc., it is important to monitor the effectiveness of the treatment and predict the patient's survival rate. Further, it is important to detect minimal residual disease of cancer cells. The present disclosure provides methods and systems for determining whether a subject has a higher survival rate after receiving a treatment for a disease, the methods and systems comprise assaying a cell-free nucleic acid molecule from at least a portion of a sample from said subject; detecting a methylation level of at least a portion of said cell-free nucleic acid molecule comprised in a differentially methylated region (DMR) listed in Table 6; and comparing, using at least one computer processor, said methylation level detected in (b) to a methylation level of corresponding portion(s) of said cell-free nucleic acid molecules comprised in said DMR listed in Table 6. In some embodiments, the DMRs listed in Table 6 represent regions associated with genes ZSCAN31, LINC01391, GATA2-AS1, STK3, and OSR1.

Table 6 ctDNA derived DMR

| | ensemblId - DMR | |
|---|---|---|
| windowPos - DMR genomic region | associated gene ID | DMR |
| chr2.19555801.19556100 | ENSG00000143867 | hyper |
| chr3.128210701.128211000 | ENSG00000179348 | hyper |
| chr3.138657301.138657600 | ENSG00000244578 | hyper |
| chr6.28303801.28304100 | ENSG00000235109 | hyper |
| chr8.99951901.99952200 | ENSG00000104375 | hyper |

In some embodiments, the method further comprises the step of adding a second amount of control DNA to the sample for confirming the immunoprecipitation reaction.

As used herein, the “control” may comprise both positive and negative control, or at least a positive control.

In some embodiments, the method further comprises the step of adding a second amount of control DNA to the sample for confirming the capture of cell-free methylated DNA.

In some embodiments, identifying the presence of DNA from cancer cells further includes identifying the cancer cell tissue of origin.

In some instances, tumor tissue sampling may be challenging or carry significant risks, in which case diagnosing and/or subtyping the cancer without the need for tumor tissue sampling may be desired. For example, lung tumor tissue sampling may require invasive procedures such as mediastinoscopy, thoracotomy, or percutaneous needle biopsy; these procedures may result in a need for hospitalization, chest tube, mechanical ventilation, antibiotics, or other medical interventions. Some individuals may not undergo the invasive procedures needed for tumor tissue sampling either because of medical comorbidities or due to preference. In some instances, the actual procedure for tumor tissue procurement may depend on the suspected cancer subtype. In other instances, cancer subtype may evolve over time within the same individual; serial assessment with invasive tumor tissue sampling procedures is often impractical and not well tolerated by patients. Thus, non-invasive cancer subtyping via blood test may have many advantageous applications in the practice of clinical oncology.

Accordingly, in some embodiments, identifying the cancer cell tissue of origin further includes identifying a cancer subtype. Preferably, the cancer subtype differentiates the cancer based on stage (e.g., early stage lung cancer treated with surgery vs late stage lung cancer treated with chemotherapy), histology (e.g., small cell carcinoma vs adenocarcinoma vs squamous cell carcinoma in lung cancer), gene expression pattern or transcription factor activity (e.g., ER status in breast cancer), copy number aberrations (e.g., HER2 status in breast cancer), specific rearrangements (e.g., FLT3 in AML), specific gene point mutational status (e.g., IDH gene point mutations), and DNA methylation patterns (e.g., MGMT gene promoter methylation in brain cancer).

In some embodiments, comparison in step (f) is carried out genome-wide.

In other embodiments, the comparison in step (f) is restricted from genome-wide to specific regulatory regions, such as, but not limited to, FANTOM5 enhancers, CpG Islands, CpG shores, CpG Shelves, or any combination of the foregoing.

In some embodiments, the methods herein are for use in the detection of the cancer.

In some embodiments, the methods herein are for use in monitoring therapy of the cancer.

Data Analysis Systems and Methods

The methods and systems disclosed herein may comprises algorithms or uses thereof. The one or more algorithms may be used to classify one or more samples from one or more subjects. The one or more algorithms may be applied to data from one or more samples. The data may comprise biomarker expression data. The methods disclosed herein may comprise assigning a classification to one or more samples from one or more subjects. Assigning the classification to the sample may comprise applying an algorithm to the methylation profile, mutation profile, and fragment length profile. In some cases, the at least one profile is inputted to a data analysis system comprising a trained algorithm for classifying the sample as obtained from a subject has a disease or minor injuries.

A data analysis system may be a trained algorithm. The algorithm may comprise a linear classifier. In some instances, the linear classifier comprises one or more of linear discriminant analysis, Fisher's linear discriminant, Naïve Bayes classifier, Logistic regression, Perceptron, Support vector machine, or a combination thereof. The linear classifier may be a support vector machine (SVM) algorithm. The algorithm may comprise a two-way classifier. The two-way classifier may comprise one or more decision tree, random forest, Bayesian network, support vector machine, neural network, or logistic regression algorithms.

The algorithm may comprise one or more linear discriminant analysis (LDA), Basic perceptron, Elastic Net, logistic regression, (Kernel) Support Vector Machines (SVM), Diagonal Linear Discriminant Analysis (DLDA), Golub Classifier, Parzen-based, (kernel) Fisher Discriminant Classifier, k-nearest neighbor, Iterative RELIEF, Classification Tree, Maximum Likelihood Classifier, Random Forest, Nearest Centroid, Prediction Analysis of Microarrays (PAM), k-medians clustering, Fuzzy C-Means Clustering, Gaussian mixture models, graded response (GR), Gradient Boosting Method (GBM), Elastic-net logistic regression, logistic regression, or a combination thereof. The algorithm may comprise a Diagonal Linear Discriminant Analysis (DLDA) algorithm. The algorithm may comprise a Nearest Centroid algorithm. The algorithm may comprise a Random Forest algorithm. In some embodiments, for discrimination of preeclampsia and non-preeclampsia, the performance of logistic regression, random forest, and gradient boosting method (GBM) is superior to that of linear discriminant analysis (LDA), neural network, and support vector machine (SVM).

Kits

The present disclosure provides kits for identifying or monitoring a disease or disorder (e.g., cancer) of a subject. A kit may comprise probes for identifying a quantitative measure (e.g., indicative of a presence, absence, or relative amount) of sequences at each of a panel of cancer-associated genomic loci in a sample of the subject. A quantitative measure (e.g., indicative of a presence, absence, or relative amount) of sequences at each of a panel of cancer-associated genomic loci in the sample may be indicative of the disease or disorder (e.g., cancer) of the subject. The probes may be selective for the sequences at the panel of cancer-associated genomic loci (e.g., DMR listed in Tables 3, 5 and 6) in the sample. A kit may comprise instructions for using the probes to process the sample to generate datasets indicative of a quantitative measure (e.g., indicative of a presence, absence, or relative amount) of sequences at each of the panel of cancer-associated genomic loci in a sample of the subject.

The probes in the kit may be selective for the sequences at the panel of cancer-associated genomic loci in the sample. The probes in the kit may be configured to selectively enrich nucleic acid (e.g., RNA or DNA) molecules corresponding to the panel of cancer-associated genomic loci. The probes in the kit may be nucleic acid primers. The probes in the kit may have sequence complementarity with nucleic acid sequences from one or more of the panel of cancer-associated genomic loci or genomic regions. The panel of cancer-associated genomic loci or microbiome-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct panel of cancer-associated genomic loci or genomic regions.

The instructions in the kit may comprise instructions to assay the sample using the probes that are selective for the sequences at the panel of cancer-associated genomic loci in the cell-free biological sample. These probes may be nucleic acid molecules (e.g., RNA or DNA) having sequence complementarity with nucleic acid sequences (e.g., RNA or DNA) from one or more of the plurality of panel of cancer-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of cancer-associated genomic loci in the sample. A quantitative measure (e.g., indicative of a presence, absence, or relative amount) of sequences at each of a panel of cancer-associated genomic loci in the sample may be indicative of a disease or disorder (e.g., cancer).

The instructions in the kit may comprise instructions to measure and interpret assay readouts, which may be quantified at one or more of the panel of cancer-associated genomic loci to generate the datasets indicative of a quantitative measure (e.g., indicative of a presence, absence, or relative amount) of sequences at each of the panel of cancer-associated genomic loci in the sample. For example, quantification of array hybridization or polymerase chain reaction (PCR) corresponding to the panel of cancer-associated genomic loci may generate the datasets indicative of a quantitative measure (e.g., indicative of apresence, absence, or relative amount) of sequences at each of the panel of cancer-associated genomic loci in the sample. Assay readouts may comprise quantitative PCR (qPCR) values, digital PCR (dPCR) values, digital droplet PCR (ddPCR) values, fluorescence values, etc., or normalized values thereof.

Computer System

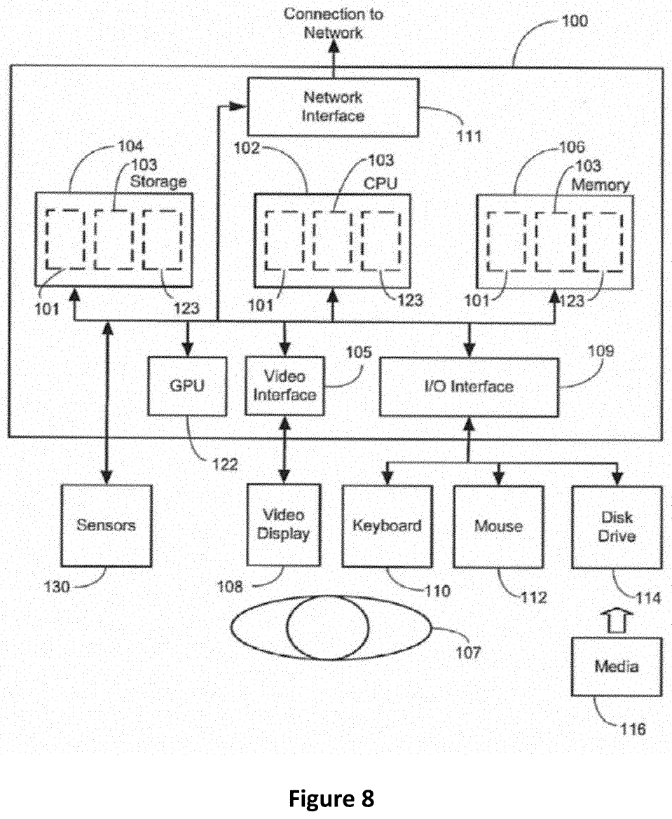

In some embodiments, certain steps are carried out by a computer processor. The present system and method may be practiced in various embodiments. A suitably configured computer device, and associated communications networks, devices, software and firmware may provide a platform for enabling one or more embodiments as described above. By way of example, FIG. 8 shows a generic computer device 100 that may include a central processing unit (“CPU”) 102 connected to a storage unit 104 and to a random access memory 106. The CPU 102 may process an operating system 101, application program 103, and data 123. The operating system 101, application program 103, and data 123 may be stored in storage unit 104 and loaded into memory 106, as may be required. Computer device 100 may further include a graphics processing unit (GPU) 122 which is operatively connected to CPU 102 and to memory 106 to offload intensive image processing calculations from CPU 102 and run these calculations in parallel with CPU 102. An operator 107 may interact with the computer device 100 using a video display 108 connected by a video interface 105, and various input/output devices such as a keyboard 115, mouse 112, and disk drive or solid state drive 114 connected by an I/O interface 109. The mouse 112 may be configured to control movement of a cursor in the video display 108, and to operate various graphical user interface (GUI) controls appearing in the video display 108 with a mouse button. The disk drive or solid state drive 114 may be configured to accept computer readable media 116. The computer device 100 may form part of a network via a network interface 111, allowing the computer device 100 to communicate with other suitably configured data processing systems (not shown). One or more different types of sensors 135 may be used to receive input from various sources.

The present system and method may be practiced on virtually any manner of computer device including a desktop computer, laptop computer, tablet computer or wireless handheld. The present system and method may also be implemented as a computer-readable/useable medium that includes computer program code to enable one or more computer devices to implement each of the various process steps in a method in accordance with the present invention. In case of more than computer devices performing the entire operation, the computer devices are networked to distribute the various steps of the operation. It is understood that the terms computer-readable medium or computer useable medium comprises one or more of any type of physical embodiment of the program code. In particular, the computer-readable/useable medium may comprise program code embodied on one or more portable storage articles of manufacture (e.g. an optical disc, a magnetic disk, a tape, etc.), on one or more data storage portioned of a computing device, such as memory associated with a computer and/or a storage system.

As used herein, “processor” may be any type of processor, such as, for example, any type of general-purpose microprocessor or microcontroller (e.g., an Intel™ x86, PowerPC™, ARM™ processor, or the like), a digital signal processing (DSP) processor, an integrated circuit, a field programmable gate array (FPGA), or any combination thereof.

As used herein “memory” may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), or the like. Portions of memory 102 may be organized using a conventional filesystem, controlled and administered by an operating system governing overall operation of a device.

As used herein, “computer readable storage medium” (also referred to as a machine-readable medium, a processor-readable medium, or a computer usable medium having a computer-readable program code embodied therein) is a medium capable of storing data in a format readable by a computer or machine. The machine-readable medium may be any suitable tangible, non-transitory medium, including magnetic, optical, or electrical storage medium including a diskette, compact disk read only memory (CD-ROM), memory device (volatile or non-volatile), or similar storage mechanism. The computer readable storage medium may contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations may also be stored on the computer readable storage medium. The instructions stored on the computer readable storage medium may be executed by a processor or other suitable processing device, and may interface with circuitry to perform the described tasks.

As used herein, “data structure” a particular way of organizing data in a computer so that it may be used efficiently. Data structures may implement one or more particular abstract data types (ADT), which specify the operations that may be performed on a data structure and the computational complexity of those operations. In comparison, a data structure is a concrete implementation of the specification provided by an ADT.

The advantages of the present invention are further illustrated by the following examples. The examples and their particular details set forth herein are presented for illustration only and should not be construed as a limitation on the claims of the present invention.

### Examples

Materials & Methods

HNSCC and Healthy Donor Peripheral Blood Leukocyte (PBL) and Plasma Acquisition

Patients diagnosed with HNSCC between 2014-2016 were identified from a prospective Anthology of Clinical Outcomes (Wong K. et al. 2010). All studies were approved by the Research Ethics Board at University Health Network. HNSCC patient samples were obtained from the Princess Margaret Cancer Centre's HNC Translational Research program based on the following criteria: 1) presentation of localized disease at diagnosis, 2) collection of blood at diagnosis and at least one timepoint post-treatment, 3) minimum follow-up time of 2 years after diagnosis. All patients received curative-intent treatment consisting of surgery with or without adjuvant radiotherapy. Healthy donors matched by age, gender, and current smoking status were identified from a prospective lung cancer screening program. 5-10 mL of blood was collected in Ethylene-Diamine-Tetraacetic Acid (EDTA) tubes. For HNSCC patients, blood was collected at diagnosis (baseline, BL) as well as three months after primary surgery (3M). Where applicable, additional blood was collected prior to adjuvant radiotherapy (PreRT), mid adjuvant radiotherapy (MidRT), and/or 12 months after primary surgery (12M). Plasma was isolated from blood within 1 hour of collection and stored at −80° C. until further processing. From the same blood collection for HNSCC patients at diagnosis or healthy donors, peripheral blood leukocytes were also isolated.

Cell Culture

The HPV-negative HNSCC cell line, FaDu, was kindly provided by Dr. Bradly Wouters (Princess Margaret Cancer Center) and cultured in DMEM (Gibco) supplemented with 10% fetal bovine serum and 1% penicillin/streptomycin. FaDu cell cultures were incubated in a humidified atmosphere containing 5% CO2 at 37° C. The identity of FaDu cells was confirmed by STR profiling. Cells were subjected to mycoplasma testing (e-MycoTMVALiD Mycoplasma PCR Detection Kit, Intron Bio) prior to use.

Isolation of Cell-Free DNA (cfDNA) and PBL Genomic DNA (gDNA)

cfDNA was isolated from total plasma using the QIAamp Circulating Nucleic Acid Kit (Qiagen) following manufacturer's instructions. Genomic DNA was isolated from PBLs, sheared to 150-200 base-pairs using the Covaris M220 Focused-ultrasonicator, and size-selected by AMPure XP magnetic beads (Beckman Coulter) to remove fragments above 300 base-pairs. Isolated cfDNA and sheared PBL genomic DNA were quantified by Qubit prior to library generation (FIGS. 9A and 9B).

Sequencing Library Preparation

5-10 or 10-20 ng of DNA was used as input for cfMeDIP-seq or CAPP-seq respectively. Input DNA was prepared for library generation using the KAPA HyperPrep Kit (KAPA Biosystems) with some modifications. Library adapters were utilized which incorporate a random 2-bp sequence followed by a constant 1-bp T sequence 5′ adjacent to both strands of input DNA upon ligation. To minimize adapter dimerization during ligation, library adapters were added at a 100:1 adapter:DNA molar ratio (˜0.07 uM per 10 ng of cfDNA) and incubated at 4° C. for 17 hours overnight. After post-ligation cleanup, input DNA was eluted in 40 uL of elution buffer (EB, 10 mM Tris-HCl, pH 8.0-8.5) prior to library generation.

Generation of CAPP-Seq Libraries

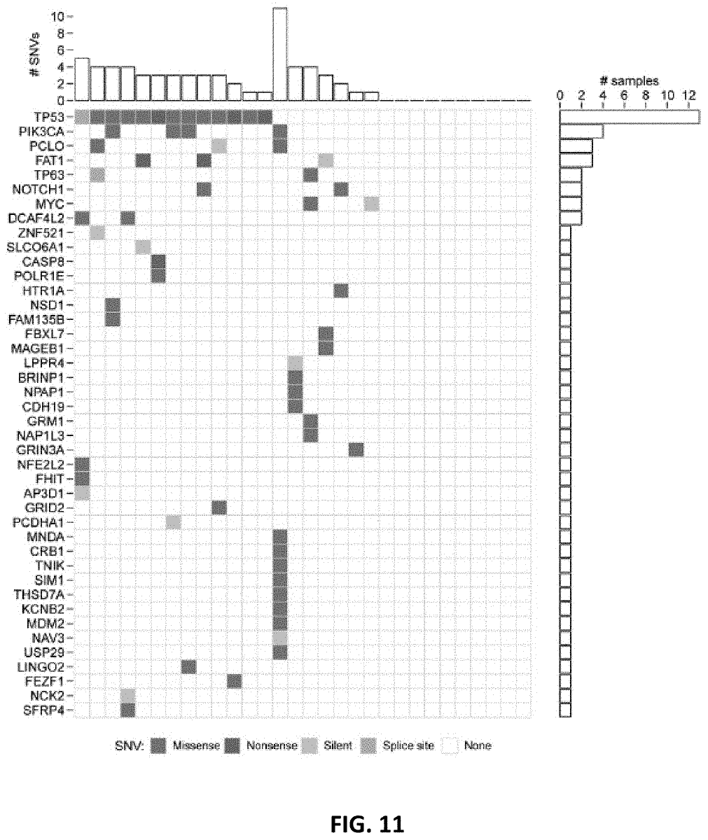

Generation of CAPP-seq libraries were performed as described from Newman et al. 2014 with some modification. Libraries were PCR amplified at 10 cycles and up to 12 indexed amplified libraries were pooled together at 500-1000 ng. After the addition of COT DNA and blocking oligos, pooled libraries underwent SpeedVac treatment to evaporate all liquids and were resuspended in 13 uL resuspension mix (8.5 uL 2× Hybridization buffer, 3.4 uL Hybridization Component A, 1.1 uL nuclease-free water). 4 uL of hybridization probes (i.e. HNSCC selector) was added to the resuspension mix for a total of 17 uL prior to hybridization. After hybridization and PCR amplification/cleanup, libraries were eluted in 30 uL of IDTE pH 8.0 (1×TE solution). Multiplexed libraries were sequenced at 2×75/100/125 paired runs on the Illumina NextSeq/NovaSeq/HiSeq4000 respectively. Design of the HNSCC selector incorporated frequently recurrent genomic alterations in HNSCC from the COSMIC database as well as the E6 and E7 region of the HPV-16 genome (FIG. 11).

Alignment and Quality Control of CAPP-Seq Libraries

The first two base-pairs on each 5′ end of unaligned paired reads, corresponding to the incorporated random molecular barcodes, were extracted and collated to generate a 4-bp molecular identifier (UMI). The third T base-pair spacer was also removed prior to alignment. Paired reads were aligned to the human genome (genome assembly GRCh37/hg19) by BWA-mem, sorted and indexed by SAMtools (v 1.3.1) and recalibrated for base quality score using the Genome Analysis ToolKit (GATK) BaseRecalibrator (v 3.8) according to best practices (reference). Duplicated sequences from BAM files were collapsed based on their UMIs and labeled as Singletons, Single-Strand Consensus Sequences (SSCS) or Duplex Consensus Sequences (DCS) by ConsensusCruncher⁴⁴. Quality control of each library was assessed by various metrics obtained form FastQC (Babraham Bioinformatics), as well as various scripts to obtain capture efficiency (CollectHsMetrics, Picard 2.10.9), depth of coverage (DepthOfCoverage, GATK 3.8), and base-pair position error rate (ides-bgreport.pl, Newman et al. 2016).

Detection of Somatic Nucleotide Variants (SNVs) and Quantification of ctDNA

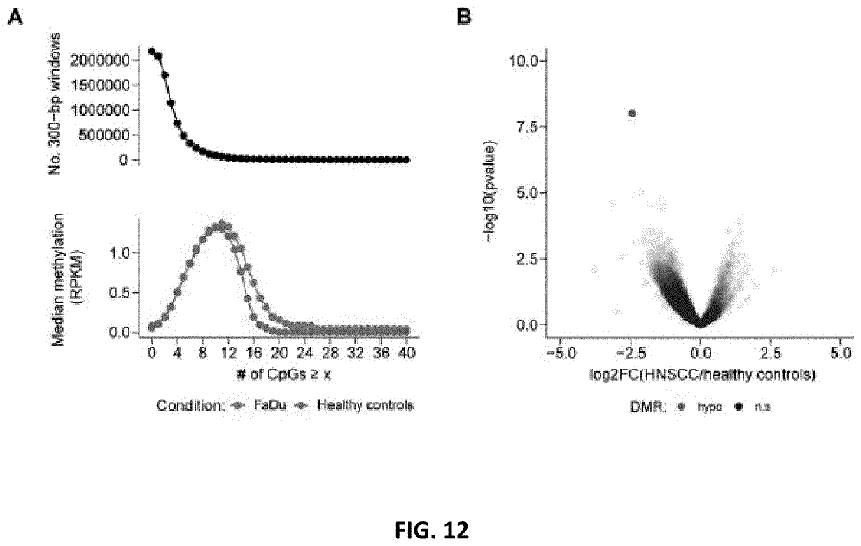

Removal of potential sequencing errors was performed by integrated Digital Error Suppression (iDES) as described by Newman et al. 2016. Background polishing was performed by utilization of our 20 healthy donor cfDNA samples as the training cohort (FIG. 12). To prevent the influence of outliers on downstream analysis, candidate SNVs within the lower 15^{th} or upper 85^{th} percentile of sequencing depth (<=1500×, >=5000×) across HNSCC cfDNA or PBL gDNA samples as well as genes with an average sequencing depth <=500× were excluded from analysis. To account for clonal hematopoiesis, non-germline mutations were defined as having a mutant allele fractions below 10% in plasma. Candidate SNVs in HNSCC cfDNA samples were identified based on the criteria of >=3 supporting reads with duplex support and complete absence in matched PBL gDNA samples. The mutant allele fraction (MAF) of identified SNVs was calculated by the number of reads corresponding to the alternative allele, divided by the sum of reads corresponding to the alternative and reference allele. For each HNSCC cfDNA sample with identifiable SNVs, the mean MAF across SNVs was calculated and used as a measure of ctDNA abundance. In cfDNA samples with only one identifiable SNV, the calculated MAF was used. Many of the detectable cancer-derived mutations may not be homozygous and may not be clonal within the tumor, and for these reasons the mean MAF may be an underestimate of the true ctDNA abundance within cell-free DNA

Generation of cfMeDIP-seq Libraries

The cfMeDIP-seq protocol was performed as described by Shen et al. 2019 with modifications to the library preparation step as described in “Sequencing Library Preparation”. Multiplexed libraries were sequenced at 2×75/100/125 paired runs on the Illumina NextSeq/NovaSeq/HiSeq4000 respectively. For generalizability, cfMeDIP-seq libraries are described as any MeDIP-seq preparation method utilizing 5-10 ng of input DNA regardless of source (i.e. cfDNA, gDNA).

Alignment and Quality Control of cfMeDIP-Seq Libraries

Unaligned paired reads were processed, aligned, sorted and indexed as previously described in Alignment and Quality Control of CAPP-seq Libraries. Duplicated sequences from BAM files were collapsed by SAMtools. Quality control of each library was assessed by various metrics obtained form FastQC (Babraham Bioinformatics), as well as various metrics obtained from the R package MEDIPS (reference) including CpG coverage (MEDIPS.seqCoverage) and enrichment (MEDIPS.CpGenrich).

Selection of Informative Regions in cfMeDIP-Seq Profiles

Fragments generated from paired reads of cfMeDIP-seq libraries were counted within non-overlapping 300 base-pair windows by MEDIPS (MEDIPS.createSet), scaled by Reads Per Kilobase per Million (RPKM), and exported as WIG format (MEDIPS.exportWIG). WIG files from each sample were imported by R and collated as a matrix. Analysis was limited to cfDNA and PBL samples from our 20 healthy donor samples to enable applications within a non-disease context. Informative regions were based on the criteria of CpG density and correlation of RPKM values between cfDNA and matched PBLs. Employing a sliding window based on CpG density (>=n CpGs), a minimum threshold of >=8 CpGs was selected.

Calculation of Absolute Methylation from cfMeDIP-Seq Libraries

Fragments from paired reads of cfMeDIP-seq libraries were counted as previously described in Selection of Informative Regions in cfMeDIP-seq Profiles and scaled to absolute methylation levels by the MeDEStrand R package. To calculate absolute methylation from counts, a logistic regression model was used to estimate bias of DNA pulldown based on CpG density (i.e. CpG density bias) (MeDEStrand.calibrationCurve). Based on the estimated CpG density bias, methylation within each window was corrected for fragments from the positive and negative DNA strand. Windows with corrected fragments were log transformed and scaled to values between 0 and 1 to describe absolute methylation (MeDEStrand.binMethyl). Absolute methylation levels from each cfMeDIP-seq sample was exported as a WIG-like file (i.e. WIG file format without a track-line).

Design of In-Silico PBL Depletion and Evaluation of Performance

To enrich for windows within the disease setting, methylation from PBLs was removed by a process termed “in-silico PBL depletion”. Analysis was limited to PBL samples from our cohort of 20 healthy donor samples to enable applications within a non-cancer specific context. Our strategy for the in-silico PBL depletion was performed as followed:
1. For each informative window as described in Selection of Informative Regions in cfMeDIP-seq Profiles, calculate the median absolute methylation value across healthy donor PBL samples.
2. Define PBL-depleted windows based on the criteria of a median absolute methylation value <0.1.
3. Restrict analysis of cfDNA samples within PBL-depleted windows.

Performance of the in-silico PBL depletion strategy was evaluated by comparing absolute methylation distributions in PBL samples before and after depletion from the healthy donor cohort used as the training set, to the HNSCC cohort used as the validation set.

Differential Methylation Analysis

To enable robust detection of HNSCC-associated differentially methylated regions (DMRs), analysis was limited to HNSCC patients with detectable SNVs in plasma by CAPP-seq (n=20/32). Differential methylation analysis was limited to informative regions after in-silico PBL depletion. A collated matrix of binned fragment counts from HNSCC and healthy donor cfDNA samples, generated as previously described in Selection of Informative Regions in cfMeDIP-seq Profiles, were utilized for identification of DMRs by the DESeq2 R package. Pre-filtering was performed by removal of regions with <10 counts across all cfDNA samples. A single factor defined as condition (HNSCC vs. healthy donor) was used for contrast during differential methylation analysis. Briefly, differential methylation analysis was performed by scaling samples based on size factors and dispersion estimates, followed by fitting of a negative binomial general linear model. For each window, a P-value was calculated between the HNSCC and healthy donor conditions by Wald Test. P-values within regions above the default Cook's distance cut-off were omitted from adjusted P-value calculation (Benjamini-Hochberg). Significant hypermethylated or hypomethylated regions (hyper-/hypo-DMRs) in HNSCC cfDNA samples are defined as windows with an adjusted P-value <0.1.

Enrichment of CpG Features within HNSCC cfDNA Hypermethylated Regions

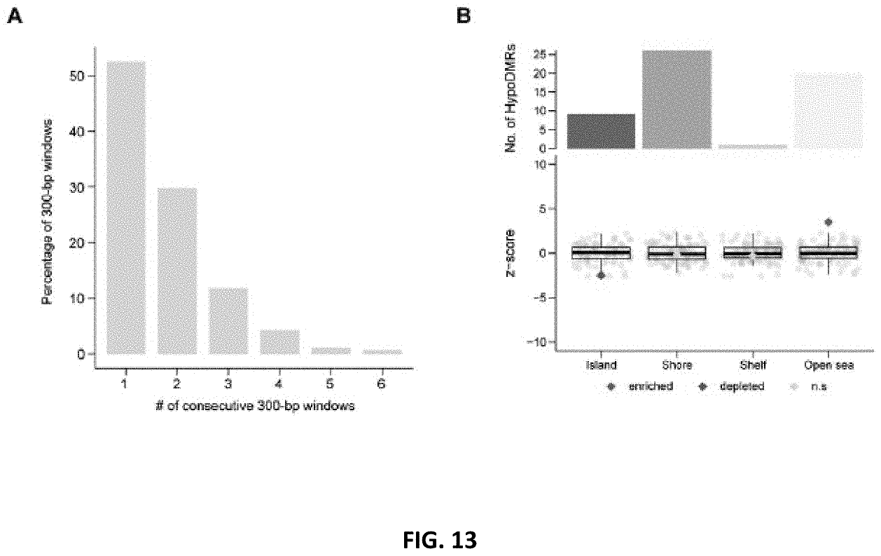

CpG features such as islands, shores, shelves, and open sea (interCGI) are defined as per the AnnotationHub R package (reference) (hg19_cpgs annotation). ID coordinates of each hypermethylated window (i.e. “chr.start.end”) within PBL-depleted regions were labeled with an overlapping CpG feature using an inhouse R package that utilizes the “annotatr” and “GenomicRanges” R packages (FIG. 13).

To determine the probability of enrichment for an observed overlap of features versus a null distribution, 1000 random samplings was performed. For each sampling, an equal number of bins were chosen based on the number hypermethylated windows, while maintaining an identical distribution of CpGs. The observed number of overlaps for each CpG feature across samplings were used to generate their respective null distributions, which were subsequently transformed onto a z-score scale. The observed overlap of hypermethylated regions for each CpG feature were also z-scored transformed, deriving summary statistics from the null distribution. The estimated P-value of the observed overlap from hypermethylated windows was calculated as the number of random samplings with overlap equal or greater/lesser than the observed overlap of the null distribution.

Enrichment of HNSCC cfDNA Hypermethylated Regions with Cancer-Specific Hypermethylated Cytosines from the Tumor Cancer Genome Atlas (TCGA)

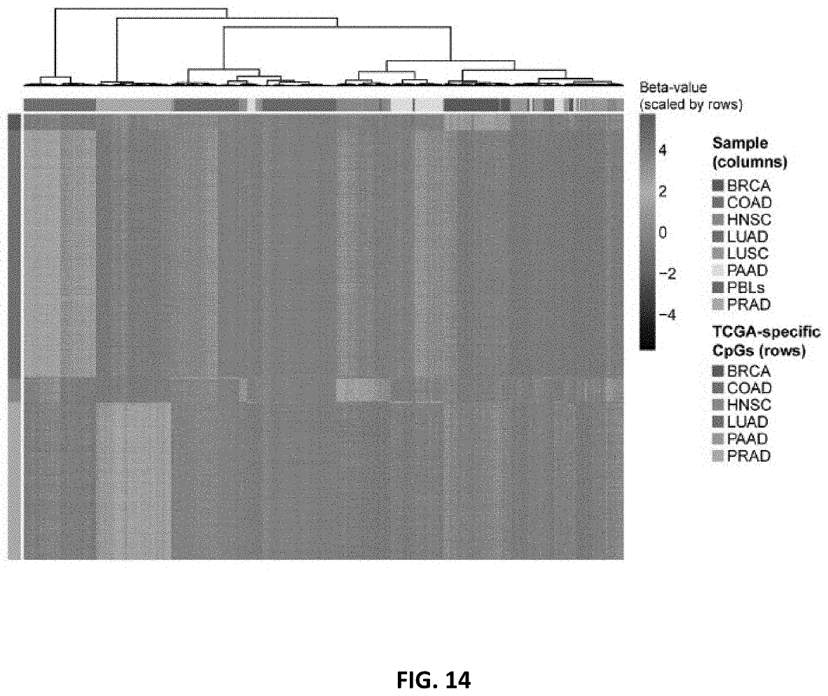

File information from publicly available hm450k profiles of all primary tumors from breast (BRCA), colorectal (COAD), head and neck (HNSC), prostate (PRAD), pancreatic (PAAD), lung adeno (LUAD), and lung squamous (LUSC) were downloaded from the TCGA. Due to the majority of our HNSCC cohort presenting with tumors of the oral cavity, files from the HNSC group were limited to patients with primary site at the “floor of mouth” (n=55). An equal number of hm450k files were randomly selected from each of the remaining cancer types, as well as from a separate database of healthy PBLs (GEO series GSE67393). A manifest of downloaded files is provided in the (FIG. 14).

To generate “tumor-specific” hyper-methylated cytosines, differential methylation analysis by limma was performed for each cancer type, with individual comparisons to each other cancer type as well as PBLs (i.e. contrast). For a given contrast, a linear model is fitted for each probed cytosine incorporating the residual variance and sample beta value, the P-value of observed difference between contrasts is then calculated by the empirical Bayes smoothing. Hypermethylated cytosines with elevated methylation in a given cancer type versus an individual comparison was defined by a log foldchange >=0.25 and an adjusted P-value (Benjamini-Hochberg)<0.01. Hypermethylated cytosines unique to an individual cancer type were designated as “tumor-specific”. For the cases of LUSC, LUAD, and PAAD, either no or very little tumor-specific hypermethylated cytosines were identified (0, 15, 18) and therefore were omitted from subsequent analysis. For comparison with cfMeDIP-seq libraries, base-pair positions from tumor-specific hypermethylated cytosines were overlapped with informative windows after in-silico PBL depletion as described in Design of In-silico PBL Depletion and Evaluation of Performance.

The enrichment of overlap for HNSCC cfDNA hypermethylated regions with tumor-specific regions from TCGA was evaluated by 10,000 random samplings using the same methods described in Enrichment of CpG Features with HNSCC cfDNA Hypermethylated Regions.

Sensitivity and Specificity of ctDNA Detection by cfMeDIP-Seq

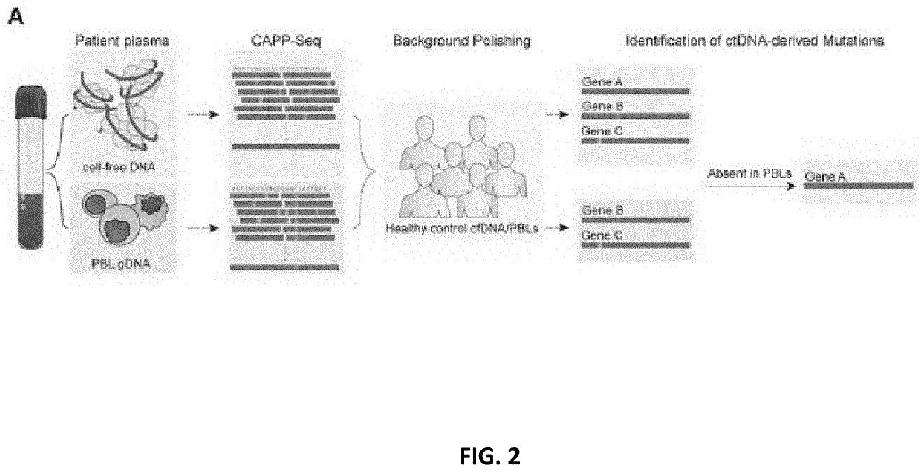

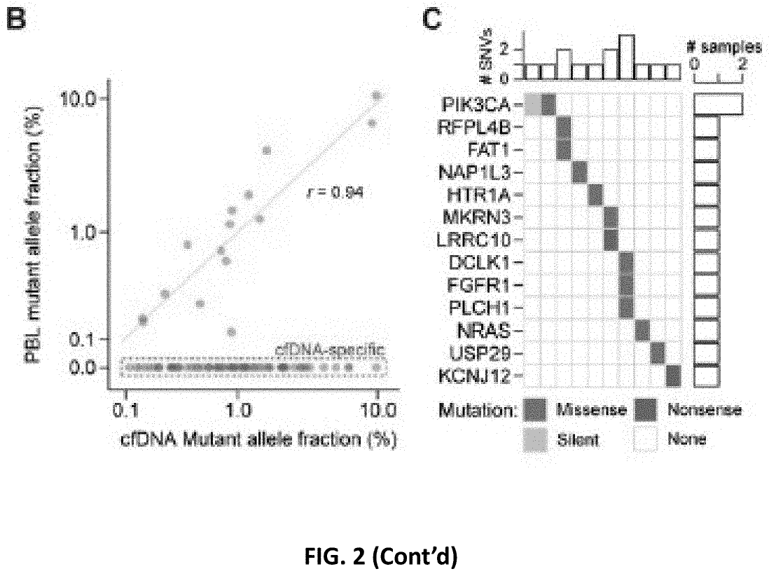

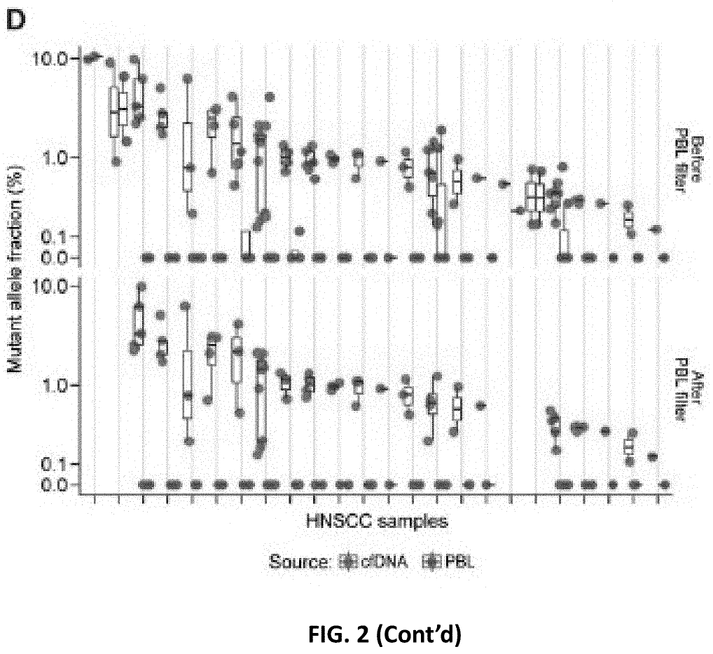

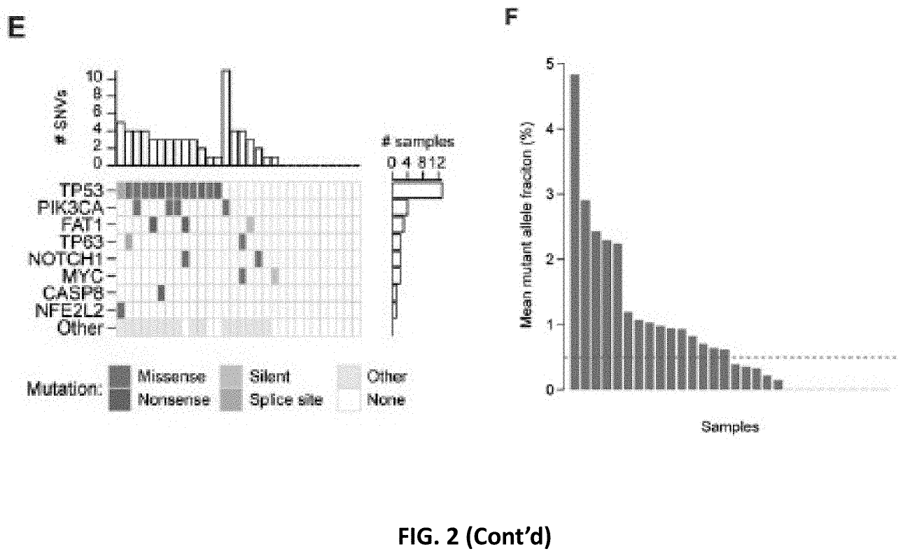

For cfMeDIP-seq libraries from our cohort of 32 HNSCC and 20 healthy donor cfDNA samples, ctDNA detection was defined based on the observation of a mean RPKM value across HNSCC cfDNA hypermethylated regions within an individual HNSCC cfDNA sample greater than the max mean RPKM value across healthy donor cfDNA samples. The sensitivity and specificity of ctDNA detection based on this definition was evaluated by Receiver Operating Characteristic (ROC) curve analysis. To minimize any confounding results due to the potential lack of ctDNA release in a subset of patients, ROC curve analysis was also performed in only 20 of the 32 HNSCC cfDNA samples with detectable ctDNA by CAPP-seq. Cross validation to assess the accuracy of ctDNA detection by DMR analysis was performed. Briefly, CAPP-Seq positive patients and healthy donors were randomly assigned to training (60%, n=24) and validation sets (40%, n=16) while maintaining similar ctDNA abundance (as determined by CAPP-Seq) between both sets. Hyper-DMRs were identified by differential methylation analysis between HNSCC and healthy donor samples within the training set. The sensitivity of ctDNA detection within these hyper-DMRs were assessed as previously described (FIG. 2C) within the validation set to obtain an AUROC value. A total of 50 random samplings were performed.

Fragment Length Analysis of ctDNA Detected by CAPP-Seq and cfMeDIP-Seq

For each HNSCC cfDNA CAPP-seq library, the median fragment length from all supporting paired reads of a specified SNV (i.e. singletons, SCSs, DCSs) as well as for paired reads containing the reference allele was measured. In cases where the median fragment length was reported for patients with >1 SNV, the median value across the median fragment length from each SNV was calculated. For each HNSCC cfDNA cfMeDIP-seq library, the median fragment length from all fragments mapping to the previously determined HNSCC cfDNA hypermethylated regions was calculated. Due to the relative absence of methylation within our cohort of 20 healthy donors, the fragment length of each healthy donor cfMeDIP-seq library was collated prior to any calculations. In both types of libraries, fragment length analysis was limited to cfDNA within the 1^{st} peak (i.e. <220 base-pairs).

Enrichment of fragments (100-150 bp or 100-220 bp) within hyper-DMRs was calculated as followed. A null distribution of expected counts was generated from random 300-bp bins within our previously designed PBL-depleted windows at identical number and CpG density distribution, from a total of 30 samplings. Observed counts for each sample were determined based on read counts across hyper-DMRs. For each sample, enrichment was calculated based on the mean observed count divided by the mean expected count.

Supervised Hierarchal Clustering

Prior to clustering, a pseudocount of 0.1 was added to all RPKM values of cfMeDIP-seq libraries to enable log 2 transformation. Values were scaled by Euclidean transformation and clustered by Ward's method. An arbitrary number of three distinct clusters were selected (k=3), designated as methylation clusters 1-3, and used in subsequent analysis.

Metrics of ctDNA Detection and Quantification on HNSCC Patient Clinical Outcomes

The potential clinical utility of ctDNA detection was evaluated by three metrics: 1) detection of SNVs by CAPP-seq, 2) detection of increased mean RPKM in hypermethylated regions by cfMeDIP-seq. For comparative analysis, patients were stratified based on the following criteria: 1) presence or absence of SNVs, 2) methylation cluster 1 vs. methylation cluster 2+3. Patient characteristics are described in Table 1.

| sampleID | pathology | smoking_status | smoking_pa [symbol] year [symbol] | age_ [symbol] | gender | dx_site | subsite | t_stage | n_stage |
|---|---|---|---|---|---|---|---|---|---|
| 1 | HNSCC | Current | 37 | 76 | Male | Lip & Oral | Tongue | T1 | N0 |
| | | | | | | Cavity | | | |
| 2 | HNSCC | Ex-smoker | 20 | 81 | Male | Paranasal | Maxillary | T3 | N0 |
| | | | | | | sinus | Sinus | | |
| 3 | HNSCC | Current | 15 | 54 | Femal [symbol] | Lip & Oral | Tongue | T2 | N2b |
| | | | | | | Cavity | | | |
| 4 | HNSCC | Ex-smoker | 20 | 63 | Male | Lip & Oral | Retromolar | T4a | N2b |
| | | | | | | Cavity | Trigone | | |
| 5 | HNSCC | Current | 30 | 47 | Male | Lip & Oral | Tongue | T4a | N2b |
| | | | | | | Cavity | | | |
| 6 | HNSCC | Current | 2 | 22 | Male | Lip & Oral | Tongue | T2 | N1 |
| | | | | | | Cavity | | | |
| 7 | HNSCC | Ex-smoker | 40 | 69 | Male | Lip & Oral | Floor of | T4a | N2c |
| | | | | | | Cavity | Mouth | | |
| 8 | HNSCC | Ex-smoker | 10 | 80 | Male | Lip & Oral | Lower | T4a | N2b |
| | | | | | | Cavity | Alveolus & | | |
| 9 | HNSCC | Current | 50 | 62 | Male | Hypopharynx | Post-cricoid | T4a | N2c |
| 10 | HNSCC | Current | 50 | 63 | Femal [symbol] | Lip & Oral | Floor of | T3 | N2c |
| | | | | | | Cavity | Mouth | | |
| 11 | HNSCC | Non-smoker | NA | 68 | Male | Lip & Oral | Lower | T4a | N2b |
| | | | | | | Cavity | Alveolus & | | |
| 12 | HNSCC | Ex-smoker | 15 | 78 | Male | Lip & Oral | Floor of | T1 | N1 |
| | | | | | | Cavity | Mouth | | |
| 13 | HNSCC | Non-smoker | NA | 53 | Male | Lip & Oral | Tongue | T2 | N2b |
| | | | | | | Cavity | | | |
| 14 | HNSCC | Ex-smoker | 5 | 59 | Femal [symbol] | Lip & Oral | Floor of | T1 | N0 |
| | | | | | | Cavity | Mouth | | |
| 15 | HNSCC | Ex-smoker | 25 | 79 | Male | Larynx | Supraglottis | T4a | N1 |
| 16 | HNSCC | Current | 55 | 74 | Male | Lip & Oral | Floor of | T2 | N0 |
| | | | | | | Cavity | Mouth | | |
| 17 | HNSCC | Current | 40 | 64 | Male | Lip & Oral | Lower | T4a | N1 |
| | | | | | | Cavity | Alveolus & | | |
| 18 | HNSCC | Current | 35 | 65 | Femal [symbol] | Lip & Oral | Floor of | T4a | N0 |
| | | | | | | Cavity | Mouth | | |
| 19 | HNSCC | Current | 30 | 52 | Femal [symbol] | Lip & Oral | Tongue | T2 | N2c |
| | | | | | | Cavity | | | |
| 20 | HNSCC | Current | 30 | 46 | Male | Larynx | Glottis | T4a | N2c |
| 21 | HNSCC | Non-smoker | NA | 46 | Male | Larynx | Supraglottis | T4a | N2b |
| 22 | HNSCC | Current | 75 | 74 | Male | Hypopharynx | Pyriform | T4a | N2c |
| 23 | HNSCC | Current | 35 | 56 | Male | Lip & Oral | Tongue | T1 | N2c |
| | | | | | | Cavity | | | |
| 24 | HNSCC | Non-smoker | NA | 33 | Male | Lip & Oral | Tongue | T1 | N0 |
| | | | | | | Cavity | | | |
| 25 | HNSCC | Non-smoker | NA | 60 | Femal [symbol] | Lip & Oral | Tongue | T1 | N1 |
| | | | | | | Cavity | | | |
| 26 | HNSCC | Current | 40 | 65 | Male | Hypopharynx | Pyriform | T3 | N0 |
| 27 | HNSCC | Current | 15 | 49 | Male | Lip & Oral | Floor of | T4a | N0 |
| | | | | | | Cavity | Mouth | | |
| 28 | HNSCC | Current | 30 | 54 | Male | Lip & Oral | Floor of | T4a | N2c |
| | | | | | | Cavity | Mouth | | |
| 29 | HNSCC | Non-smoker | NA | 54 | Male | Lip & Oral | Tongue | T1 | N0 |
| | | | | | | Cavity | | | |
| 30 | HNSCC | Current | 30 | 56 | Male | Lip & Oral | Floor of | T2 | N0 |
| | | | | | | Cavity | Mouth | | |
| 1 | Healthy dono [symbol] | Ex-smoker | 35 | 69 | Male | NA | NA | NA | NA |
| 2 | Healthy dono [symbol] | Current | 84 | 74 | Male | NA | NA | NA | NA |
| 3 | Healthy dono [symbol] | Ex-smoker | 40 | 77 | Male | NA | NA | NA | NA |
| 4 | Healthy dono [symbol] | Non-smoker | NA | 82 | Male | NA | NA | NA | NA |
| 5 | Healthy dono [symbol] | Ex-smoker | 14 | 61 | Male | NA | NA | NA | NA |
| 6 | Healthy dono [symbol] | Current | 55 | 71 | Male | NA | NA | NA | NA |
| 7 | Healthy dono [symbol] | Current | 50 | 65 | Male | NA | NA | NA | NA |
| 8 | Healthy dono [symbol] | Ex-smoker | 30 | 69 | Male | NA | NA | NA | NA |
| 9 | Healthy dono [symbol] | Ex-smoker | 41 | 57 | Femal [symbol] | NA | NA | NA | NA |
| 10 | Healthy dono [symbol] | Current | 10 | 81 | Male | NA | NA | NA | NA |
| 11 | Healthy dono [symbol] | Current | 39 | 64 | Femal [symbol] | NA | NA | NA | NA |
| 12 | Healthy dono [symbol] | Ex-smoker | 30 | 65 | Femal [symbol] | NA | NA | NA | NA |
| 13 | Healthy dono [symbol] | Current | 17.5 | 64 | Male | NA | NA | NA | NA |
| 14 | Healthy dono [symbol] | Ex-smoker | 50 | 77 | Male | NA | NA | NA | NA |
| 15 | Healthy dono [symbol] | Ex-smoker | 10 | 59 | Femal [symbol] | NA | NA | NA | NA |
| 16 | Healthy dono [symbol] | Non-smoker | NA | 64 | Male | NA | NA | NA | NA |
| 17 | Healthy dono [symbol] | Ex-smoker | 20 | 66 | Male | NA | NA | NA | NA |
| 18 | Healthy dono [symbol] | Ex-smoker | 24.75 | 60 | Femal [symbol] | NA | NA | NA | NA |
| 19 | Healthy dono [symbol] | Ex-smoker | 15 | 56 | Male | NA | NA | NA | NA |
| 20 | Healthy dono [symbol] | Non-smoker | NA | 83 | Male | NA | NA | NA | NA |

| sampleID | m_stage | clinical_stage | hpv_status | chemotherapy | treatment | vital_status | cause_of_death | relapse |
|---|---|---|---|---|---|---|---|---|
| 1 | M0 | I | NA | No | Sx only | Alive | NA | No |
| 2 | M0 | III | Negative | No | post-op | Dead | Cancer | Yes |
| 3 | M0 | IVA | NA | Yes | post-op C [symbol] | Alive | NA | No |
| 4 | M0 | IVA | NA | No | post-op | Alive | NA | No |
| 5 | M0 | IVA | Negative | No | post-op | Dead | Cancer | Yes |
| 6 | M0 | III | NA | Yes | post-op C [symbol] | Dead | Cancer | Yes |
| 7 | M0 | IVA | NA | No | post-op | Alive | NA | No |
| 8 | M0 | IVA | NA | No | post-op | Dead | Cancer | Yes |
| 9 | M0 | IVA | Negative | No | post-op | Dead | Cancer | Yes |
| 10 | M0 | IVA | NA | Yes | post-op C [symbol] | Dead | Index Cancer | Yes |
| 11 | M0 | IVA | NA | Yes | post-op C [symbol] | Dead | Cancer | Yes |
| 12 | M0 | III | NA | No | Sx only | Alive | NA | No |
| 13 | M0 | IVA | Negative | Yes | post-op C [symbol] | Alive | NA | No |
| 14 | M0 | I | NA | No | Sx only | Alive | NA | No |
| 15 | M0 | IVA | Negative | No | post-op | Alive | NA | No |
| 16 | M0 | II | NA | No | Sx only | Dead | Unknown | No |
| 17 | M0 | IVA | NA | Yes | post-op C [symbol] | Alive | NA | No |
| 18 | M0 | IVA | NA | No | post-op | Alive | NA | No |
| 19 | M0 | IVA | Negative | Yes | post-op C [symbol] | Alive | NA | No |
| 20 | M0 | IVA | Negative | Yes | post-op C [symbol] | Alive | NA | Yes |
| 21 | M0 | IVA | Negative | No | post-op | Alive | NA | No |
| 22 | M0 | IVA | NA | No | post-op | Dead | Cancer | Yes |
| 23 | M0 | IVA | NA | Yes | post-op C [symbol] | Alive | NA | No |
| 24 | M0 | I | NA | No | Sx only | Alive | NA | No |
| 25 | M0 | III | NA | Yes | post-op C [symbol] | Alive | NA | No |
| 26 | M0 | III | NA | No | post-op | Dead | Unknown | No |
| 27 | M0 | IVA | NA | No | post-op | Alive | NA | No |
| 28 | M0 | IVA | NA | Yes | post-op C [symbol] | Alive | NA | No |
| 29 | M0 | I | Negative | No | post-op | Alive | NA | No |
| 30 | M0 | II | NA | No | post-op | Alive | NA | No |
| 1 | NA | NA | NA | NA | NA | NA | NA | NA |
| 2 | NA | NA | NA | NA | NA | NA | NA | NA |
| 3 | NA | NA | NA | NA | NA | NA | NA | NA |
| 4 | NA | NA | NA | NA | NA | NA | NA | NA |
| 5 | NA | NA | NA | NA | NA | NA | NA | NA |
| 6 | NA | NA | NA | NA | NA | NA | NA | NA |
| 7 | NA | NA | NA | NA | NA | NA | NA | NA |
| 8 | NA | NA | NA | NA | NA | NA | NA | NA |
| 9 | NA | NA | NA | NA | NA | NA | NA | NA |
| 10 | NA | NA | NA | NA | NA | NA | NA | NA |
| 11 | NA | NA | NA | NA | NA | NA | NA | NA |
| 12 | NA | NA | NA | NA | NA | NA | NA | NA |
| 13 | NA | NA | NA | NA | NA | NA | NA | NA |
| 14 | NA | NA | NA | NA | NA | NA | NA | NA |
| 15 | NA | NA | NA | NA | NA | NA | NA | NA |
| 16 | NA | NA | NA | NA | NA | NA | NA | NA |
| 17 | NA | NA | NA | NA | NA | NA | NA | NA |
| 18 | NA | NA | NA | NA | NA | NA | NA | NA |
| 19 | NA | NA | NA | NA | NA | NA | NA | NA |
| 20 | NA | NA | NA | NA | NA | NA | NA | NA |

[symbol]

indicates data missing or illegible when filed

Cross-Validation of ctDNA-Derived Methylation by cfMeDIP-Seq Analysis

To evaluate the robustness of cfMeDIP-seq for identifying ctDNA-derived methylation, Receiver Operating Characteristics (ROC) curve analysis was performed. To minimize confounding results due to low/absent ctDNA, analysis was limited to HNSCC patients with detectable ctDNA by CAPP-seq. Patient and healthy control cfMeDIP-seq profiles were split into a training set (HNSCC: n=12/20; healthy control: n=12/20) and testing set (HNSCC: n=8/20; healthy control: n=8/20). Training and testing sets were balanced for ctDNA abundance as determined by CAPP-Seq analysis. A total of 50 splits were performed with ROC curve analysis performed on each iteration.

Identification of Prognostic Regions in HNSCC by TCGA Analysis

All available HNSCC cases from TCGA with matched legacy hm450k and RNA expression data were selected (n=520). Survival data was obtained from Jianfang et al. With regards to the hm450k data, methylation was summarized to 300-bp regions as described previously by calculating the mean beta-value between probe IDs within a particular region. To identify regions hypermethylated in HNSCC primary tumors compared to adjacent normal tissue, independent Wilcoxon tests were performed for each region. Regions with an adjusted p-value <0.05 (Holms method) as well as a log-fold change >=1 in primary tumors compared to adjacent normal tissue, were selected for subsequent analysis. To identify hypermethylated regions associated with prognosis, multivariate Cox Regression was performed, considering age, gender, and clinical stage, selecting regions with p-values <0.05. Survival analysis was limited to a maximum follow-up time of 5 years post-diagnosis, reflecting what was observed within the HNSCC cfDNA cohort. To further identify prognostic regions associated with changes in gene expression, Spearman's correlation was calculated for hm450k primary tumor profiles for each region, to matched RNA expression profiles for transcripts within a 2-Kb window. Regions with absolute Rho values >0.3 and a false discovery rate <0.05 were selected, resulting in the final identification of 5 prognostic regions associated with ZNF323/ZSCAN31, LINC01395, GATA2-AS1, OSR1, and STK3/MST2 expression. For TCGA patient profiles, the Composite Methylation Score (CMS) was obtained by calculating the sum of beta-values across all 5 prognostic regions. For cfMeDIP-seq profiles, RPKM values across all 943 hyper-DMRs were scaled to a total sum of 1 and the CMS was obtained by calculating the sum of these scaled RPKM values across all 5 prognostic regions.

Longitudinal Monitoring of Post-Treatment Plasma Samples by cfMeDIP-Seq

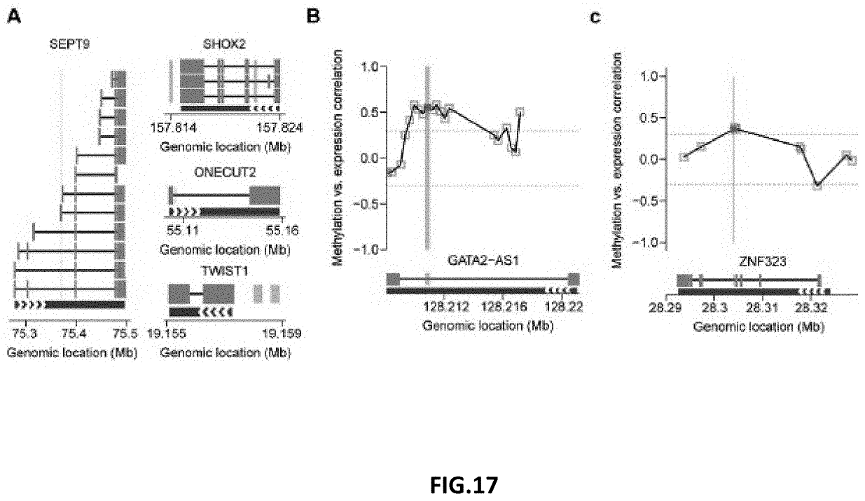

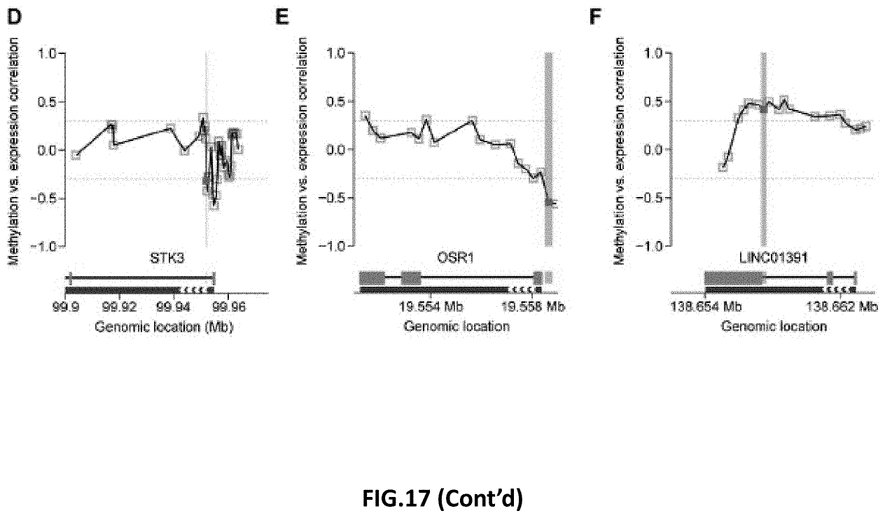

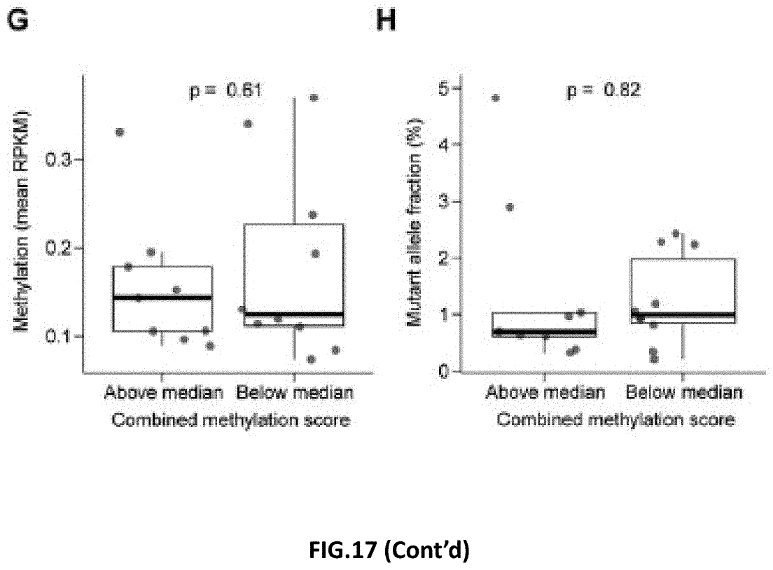

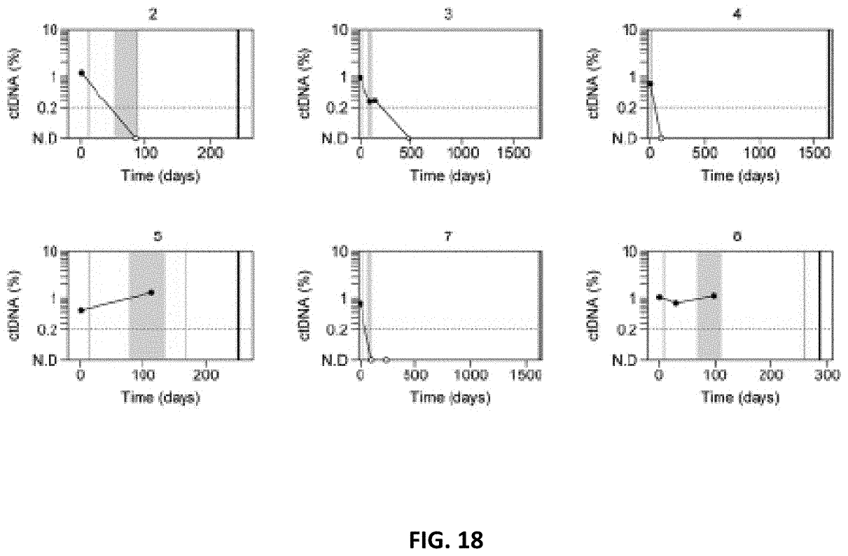

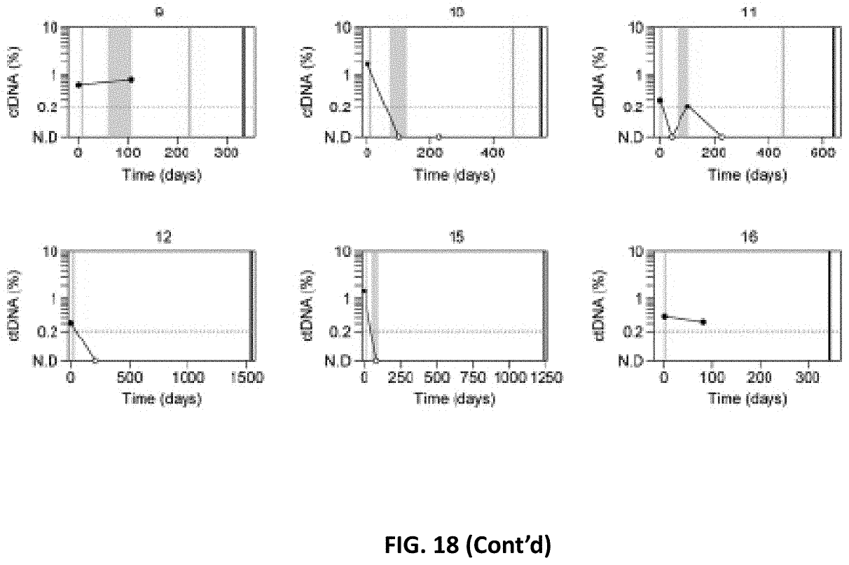

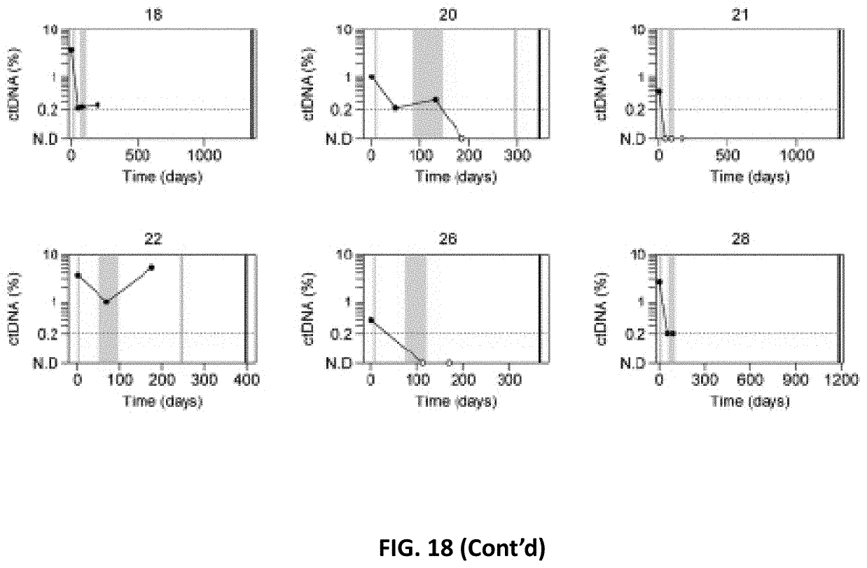

cfMeDIP-seq libraries were successfully generated for 30/32 patients (FIGS. 17A-17D). For the remaining two patients, insufficient material was isolated from plasma and/or did not pass quality metrics. ctDNA quantification of post-treatment cfMeDIP-seq libraries was performed as previously described, calculating the mean RPKM values across identified hypermethylated regions by differential methylation analysis. For ease on interpretation, both pre-treatment and post-treatment cfMeDIP-seq libraries were converted to percent DNA values based on linear regression against mean MAF calculated by matched CAPP-Seq profiles. To achieve high confidence detection of residual disease, a minimum ctDNA fraction of 0.2% was required in post-treatment samples, corresponding to the maximum of mean RPKM values observed across all healthy controls.

Results & Discussion

Multimodal Profiling of Cell-Free DNA in Localized HNSCC

To examine the ability of multimodal profiling to characterize ctDNA in the setting of localized cancer, we recruited 32 HNSCC patients into a prospective observational study in which peripheral blood samples were collected at serial timepoints (FIG. 9A; Table 1). All patients were treated with surgery, with a subset receiving adjuvant radiotherapy (n=14) or chemoradiotherapy (n=11). With a median follow up of 43.2 months, 9/32 patients (28%) developed recurrence (actuarial 2-year recurrence-free survival: 88%).

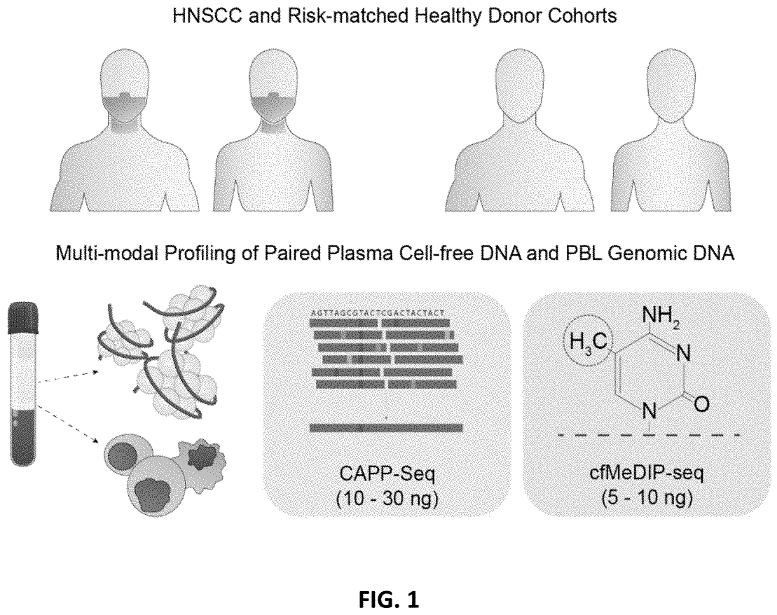

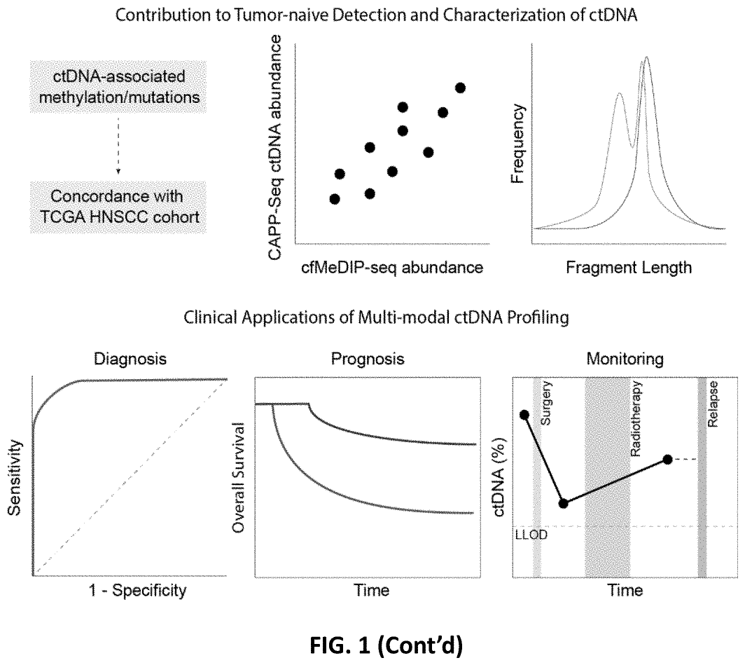

As the majority of patients exhibited a heavy smoking history, which is well-described to alter the genomic/epigenomic landscape of somatic tissue and contribute to premalignant lesions, we also analyzed blood samples from 20 risk-matched healthy donors previously enrolled in a lung cancer screening program³⁴⁻³⁷. Cell-free DNA from plasma as well as genomic DNA (gDNA) from PBLs were co-isolated from blood and subjected to quantification and analysis (Supplementary FIG. 1A). In contrast to other studies that have demonstrated significantly elevated levels of total plasma cell-free DNA in metastatic disease compared to healthy controls³⁸⁻⁴¹, no significant difference was observed between our HNSCC cohort and healthy donors (Supplementary FIG. 1B).

Multimodal profiling of cell-free DNA and PBL gDNA from patients and healthy controls were conducted (FIG. 1). By subjecting the same samples to both mutation and methylome profiling, we were able to evaluate their contributions to tumor-naïve detection and characterization of ctDNA. Mutations and methylation were independently profiled using CAncer Personalized Profiling by deep Sequencing (CAPP-Seq) and cell-free Methylated DNA ImmunoPrecipitation and high-throughput sequencing (cfMeDIP-seq), respectively. In addition, paired-end sequencing was utilized for both methodologies in order to obtain the lengths of sequenced cell-free DNA fragments.

Tumor-Naïve Detection of Mutation-Based ctDNA from Pre-Treatment Plasma

We first evaluated approaches to improve our confidence of mutation-based ctDNA detection without confirmation within matched tumor samples. Recent studies have illustrated that genes frequently targeted for ctDNA detection, such as TP53, can harbor mutations derived from clonally expanded PBLs. Additionally, as ctDNA contains both genetic and epigenetic features of the tumor, we reasoned that orthogonal analysis of both features in patient cell-free DNA may provide increased confidence of ctDNA detection. Therefore, to achieve tumor-naïve detection of low-abundance ctDNA with high confidence, mutations and methylation were independently profiled by CAPP-Seq and cfMeDIP-seq, respectively, for both cfDNA and matched PBLs.

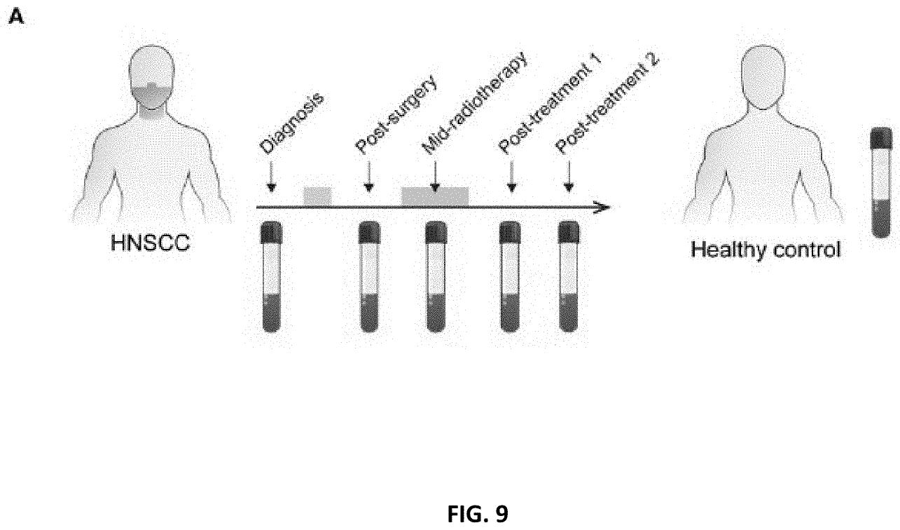

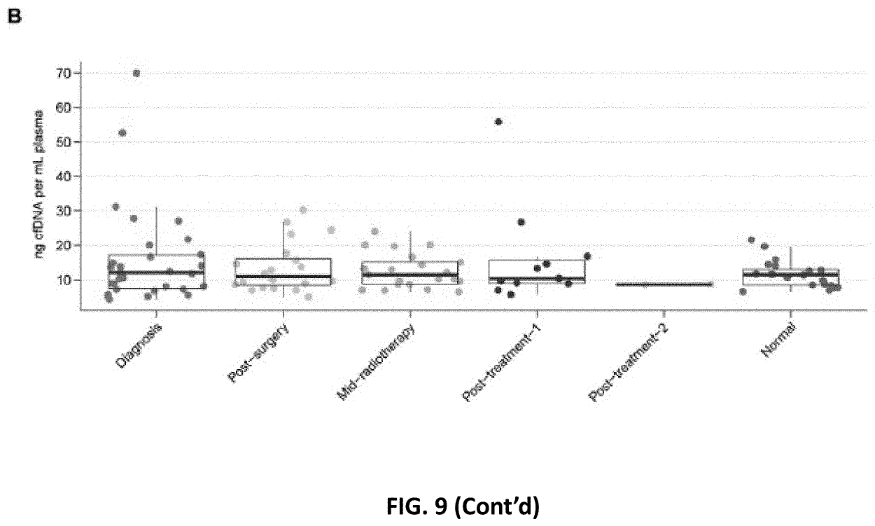

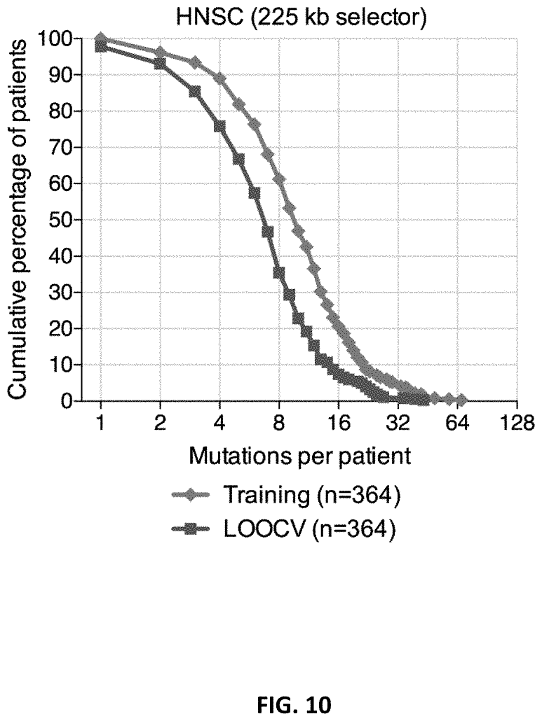

To evaluate the sensitivity of ctDNA detection in HPV-negative THNSCC without prior knowledge from the tumor, we first measured the abundance of mutations in baseline plasma samples (FIG. 2A). CAPP-Seq was conducted with a sequencing panel designed to maximize the number of HNSCC-associated mutations (Table 3 and FIG. 10). We also employed established error suppression methodologies to remove background base substitution errors.

TABLE 3
Targeted genomic regions of HNSCC CAPP-Seq selector

| Chr | Start | End | Length | Exon | Strand | Gene_canonical | Transcript |
|---|---|---|---|---|---|---|---|
| chr1 | 16464346 | 16464592 | 247 | 5 | − | EPHA2 | NM_004431.4 |
| chr1 | 16475056 | 16475543 | 488 | 3 | − | EPHA2 | NM_004431.4 |
| chr1 | 22838527 | 22838627 | 101 | 11 | + | ZBTB40 | NM_014870.3 |
| chr1 | 38227190 | 38227732 | 543 | 3 | − | EPHA10 | NM_001099439.1 |
| chr1 | 57257787 | 57258322 | 536 | 2 | − | C1orf168 | NM_001004303.4 |
| chr1 | 57480678 | 57480885 | 208 | 14 | − | DAB1 | NM_021080.3 |
| chr1 | 74575077 | 74575237 | 161 | 5 | − | LRRIQ3 | NM_001105659.1 |
| chr1 | 89616097 | 89616258 | 162 | 6 | − | GBP7 | NM_207398.2 |
| chr1 | 97547897 | 97547997 | 101 | 22 | − | DPYD | NM_000110.3 |
| chr1 | 97770870 | 97770970 | 101 | 18 | − | DPYD | NM_000110.3 |
| chr1 | 98165041 | 98165141 | 101 | 6 | − | DPYD | NM_000110.3 |
| chr1 | 99771257 | 99772532 | 1276 | 7 | + | LPPR4 | NM_014839.4 |
| chr1 | 103471370 | 103471470 | 101 | 18 | − | COL11A1 | NM_001854.3 |
| chr1 | 103480058 | 103480158 | 101 | 13 | − | COL11A1 | NM_001854.3 |
| chr1 | 115251151 | 115251275 | 125 | 5 | − | NRAS | NM_002524.4 |
| chr1 | 115252189 | 115252349 | 161 | 4 | − | NRAS | NM_002524.4 |
| chr1 | 115256420 | 115256599 | 180 | 3 | − | NRAS | NM_002524.4 |
| chr1 | 115258670 | 115258798 | 129 | 2 | − | NRAS | NM_002524.4 |
| chr1 | 119575732 | 119575894 | 163 | 6 | − | WARS2 | NM_015836.3 |
| chr1 | 149859079 | 149859463 | 385 | 1 | − | HIST2H2AB | NM_175065.2 |
| chr1 | 149905298 | 149905423 | 126 | 10 | − | MTMR11 | NM_001145862.1 |
| chr1 | 154898829 | 154898959 | 131 | 4 | − | PMVK | NM_006556.3 |
| chr1 | 157555965 | 157556231 | 267 | 6 | − | FCRL4 | NM_031282.2 |
| chr1 | 157557066 | 157557332 | 267 | 5 | − | FCRL4 | NM_031282.2 |
| chr1 | 158152697 | 158152797 | 101 | 5 | + | CD1D | NM_001766.3 |
| chr1 | 158326517 | 158326686 | 170 | 6 | + | CD1E | NM_030893.3 |
| chr1 | 158817522 | 158817653 | 132 | 6 | + | MNDA | NM_002432.1 |
| chr1 | 161479695 | 161479795 | 101 | 4 | + | FCGR2A | NM_001136219.1 |
| chr1 | 161514492 | 161514592 | 101 | 4 | − | FCGR3A | NM_000569.7 |
| chr1 | 169390627 | 169391473 | 847 | 3 | − | CCDC181 | NM_021179.2 |
| chr1 | 169578748 | 169578892 | 145 | 8 | − | SELP | NM_003005.3 |
| chr1 | 190067262 | 190068200 | 939 | 8 | − | FAM5C | NM_199051.2 |
| chr1 | 196309568 | 196309668 | 101 | 16 | − | KCNT2 | NM_198503.3 |
| chr1 | 197390164 | 197391037 | 874 | 6 | + | CRB1 | NM_201253.2 |
| chr1 | 198713182 | 198713332 | 151 | 26 | + | PTPRC | NM_002838.4 |
| chr1 | 205038983 | 205039124 | 142 | 18 | + | CNTN2 | NM_005076.4 |
| chr1 | 205779302 | 205779509 | 208 | 2 | − | SLC41A1 | NM_173854.5 |
| chr1 | 207010005 | 207010151 | 147 | 2 | + | IL19 | NM_153758.2 |
| chr1 | 215847663 | 215848873 | 1211 | 63 | − | USH2A | NM_206933.2 |
| chr1 | 216850475 | 216850747 | 273 | 2 | − | ESRRG | NM_001438.3 |
| chr1 | 248028042 | 248028156 | 115 | 3 | + | TRIM58 | NM_015431.3 |
| chr2 | 1459847 | 1460008 | 162 | 7 | + | TPO | NM_000547.5 |
| chr2 | 15415719 | 15415924 | 206 | 44 | − | NBAS | NM_015909.3 |
| chr2 | 27121416 | 27121557 | 142 | 2 | + | DPYSL5 | NM_020134.3 |
| chr2 | 28634819 | 28634952 | 134 | 4 | + | FOSL2 | NM_005253.3 |
| chr2 | 51254660 | 51255257 | 598 | 2 | − | NRXN1 | NM_004801.5 |
| chr2 | 65540892 | 65541048 | 157 | 6 | − | SPRED2 | NM_181784.2 |
| chr2 | 75720459 | 75720730 | 272 | 4 | − | EVA1A | NM_001135032.1 |
| chr2 | 79349113 | 79349251 | 139 | 4 | + | REG1A | NM_002909.4 |
| chr2 | 80136776 | 80136880 | 105 | 7 | + | CTNNA2 | NM_004389.3 |
| chr2 | 80529667 | 80530938 | 1272 | | + | CTNNA2 | NM_004389.3 |
| chr2 | 90249111 | 90249392 | 282 | | + | abParts | |
| chr2 | 99012565 | 99013683 | 1119 | 8 | + | CNGA3 | NM_001298.2 |
| chr2 | 106497875 | 106498430 | 556 | 3 | + | NCK2 | NM_001004720.2 |
| chr2 | 133402677 | 133403123 | 447 | 2 | + | GPR39 | NM_001508.2 |
| chr2 | 138169278 | 138169409 | 132 | 14 | + | THSD7B | NM_001316349.1 |
| chr2 | 141081496 | 141081596 | 101 | 81 | − | LRP1B | NM_018557.2 |
| chr2 | 141359045 | 141359175 | 131 | 42 | − | LRP1B | NM_018557.2 |
| chr2 | 141806555 | 141806673 | 119 | 11 | − | LRP1B | NM_018557.2 |
| chr2 | 160206240 | 160206346 | 107 | 28 | − | BAZ2B | NM_013450.3 |
| chr2 | 164466119 | 164468153 | 2035 | 3 | − | FIGN | NM_018086.3 |
| chr2 | 166003407 | 166003510 | 104 | 12 | − | SCN3A | NM_006922.3 |
| chr2 | 176958040 | 176958203 | 164 | 1 | + | HOXD13 | NM_000523.3 |
| chr2 | 178095512 | 178096736 | 1225 | 5 | − | NFE2L2 | NM_006164.4 |
| chr2 | 178097119 | 178097311 | 193 | 4 | − | NFE2L2 | NM_006164.4 |
| chr2 | 178097972 | 178098072 | 101 | 3 | − | NFE2L2 | NM_006164.4 |
| chr2 | 178098732 | 178098999 | 268 | 2 | − | NFE2L2 | NM_006164.4 |
| chr2 | 178129232 | 178129332 | 101 | 1 | − | NFE2L2 | NM_006164.4 |
| chr2 | 198948735 | 198950751 | 2017 | 2 | + | PLCL1 | NM_006226.3 |
| chr2 | 202122944 | 202123115 | 172 | 1 | + | CASP8 | NM_001080125.1 |
| chr2 | 202131173 | 202131524 | 352 | 2 | + | CASP8 | NM_001080125.1 |
| chr2 | 202134222 | 202134338 | 117 | | + | CASP8 | NM_001080125.1 |
| chr2 | 202136214 | 202136354 | 141 | 3 | + | CASP8 | NM_001080125.1 |
| chr2 | 202137349 | 202137509 | 161 | 4 | + | CASP8 | NM_001080125.1 |
| chr2 | 202137593 | 202137693 | 101 | 5 | + | CASP8 | NM_001080125.1 |
| chr2 | 202139594 | 202139694 | 101 | 6 | + | CASP8 | NM_001080125.1 |
| chr2 | 202140209 | 202140309 | 101 | | + | CASP8 | NM_001080125.1 |
| chr2 | 202141539 | 202141702 | 164 | 7 | + | CASP8 | NM_001080125.1 |
| chr2 | 202142434 | 202142538 | 105 | | + | CASP8 | NM_001080125.1 |
| chr2 | 202149528 | 202150050 | 523 | 8 | + | CASP8 | NM_001080125.1 |
| chr2 | 202151171 | 202151327 | 157 | 9 | + | CASP8 | NM_001080125.1 |
| chr2 | 202152123 | 202152223 | 101 | 9 | + | CASP8 | NM_001080125.1 |
| chr2 | 213878514 | 213878658 | 145 | 7 | − | IKZF2 | NM_016260.2 |
| chr2 | 217124225 | 217124379 | 155 | 4 | − | MARCH4 | NM_020814.2 |
| chr2 | 225338951 | 225339103 | 153 | 16 | − | CUL3 | NM_003590.4 |
| chr2 | 225342906 | 225343072 | 167 | 15 | − | CUL3 | NM_003590.4 |
| chr2 | 225346598 | 225346805 | 208 | 14 | − | CUL3 | NM_003590.4 |
| chr2 | 225360538 | 225360693 | 156 | 13 | − | CUL3 | NM_003590.4 |
| chr2 | 225362459 | 225362576 | 118 | 12 | − | CUL3 | NM_003590.4 |
| chr2 | 225365069 | 225365214 | 146 | 11 | − | CUL3 | NM_003590.4 |
| chr2 | 225367671 | 225367799 | 129 | 10 | − | CUL3 | NM_003590.4 |
| chr2 | 225368358 | 225368549 | 192 | 9 | − | CUL3 | NM_003590.4 |
| chr2 | 225370662 | 225370859 | 198 | 8 | − | CUL3 | NM_003590.4 |
| chr2 | 225371564 | 225371730 | 167 | 7 | − | CUL3 | NM_003590.4 |
| chr2 | 225376060 | 225376309 | 250 | 6 | − | CUL3 | NM_003590.4 |
| chr2 | 225378230 | 225378365 | 136 | 5 | − | CUL3 | NM_003590.4 |
| chr2 | 225379318 | 225379499 | 182 | 4 | − | CUL3 | NM_003590.4 |
| chr2 | 225400234 | 225400368 | 135 | 3 | − | CUL3 | NM_003590.4 |
| chr2 | 225422365 | 225422583 | 219 | 2 | − | CUL3 | NM_003590.4 |
| chr2 | 225427900 | 225428047 | 148 | | − | CUL3 | NM_003590.4 |
| chr2 | 225431632 | 225431732 | 101 | | − | CUL3 | NM_003590.4 |
| chr2 | 225434395 | 225434499 | 105 | | − | CUL3 | NM_003590.4 |
| chr2 | 225449643 | 225449743 | 101 | 1 | − | CUL3 | NM_003590.4 |
| chr2 | 227924129 | 227924320 | 192 | 28 | − | COL4A4 | NM_000092.4 |
| chr3 | 6903212 | 6903557 | 346 | 1 | + | GRM7 | NM_181874.2 |
| chr3 | 12046075 | 12046175 | 101 | 1 | + | SYN2 | NM_133625.4 |
| chr3 | 30691783 | 30691948 | 166 | 4 | + | TGFBR2 | NM_001024847.2 |
| chr3 | 30732941 | 30733073 | 133 | 8 | + | TGFBR2 | NM_001024847.2 |
| chr3 | 49412866 | 49413022 | 157 | 2 | − | RHOA | NM_001664.3 |
| chr3 | 59737949 | 59738049 | 101 | 9 | − | FHIT | NM_001166243.2 |
| chr3 | 59908056 | 59908156 | 101 | 8 | − | FHIT | NM_001166243.2 |
| chr3 | 59997061 | 59997161 | 101 | 7 | − | FHIT | NM_001166243.2 |
| chr3 | 59999732 | 59999878 | 147 | 6 | − | FHIT | NM_001166243.2 |
| chr3 | 60522592 | 60522695 | 104 | 5 | − | FHIT | NM_001166243.2 |
| chr3 | 109028016 | 109028177 | 162 | 4 | − | DPPA2 | NM_138815.3 |
| chr3 | 109049478 | 109049642 | 165 | 5 | − | DPPA4 | NM_018189.3 |
| chr3 | 115395072 | 115395172 | 101 | 3 | + | GAP43 | NM_001130064.1 |
| chr3 | 124418715 | 124418880 | 166 | 56 | + | KALRN | NM_001024660.4 |
| chr3 | 126707543 | 126708597 | 1055 | 1 | + | PLXNA1 | NM_032242.3 |
| chr3 | 129370517 | 129370617 | 101 | 6 | − | TMCC1 | NM_001017395.3 |
| chr3 | 132435600 | 132435753 | 154 | 4 | − | NPHP3 | NM_153240.4 |
| chr3 | 140167411 | 140167511 | 101 | 6 | + | CLSTN2 | NM_022131.2 |
| chr3 | 145912910 | 145913069 | 160 | 8 | − | PLSCR4 | NM_020353.2 |
| chr3 | 147108778 | 147109008 | 231 | 4 | − | ZIC4 | NM_001168378.1 |
| chr3 | 147113699 | 147114241 | 543 | 3 | − | ZIC4 | NM_001168378.1 |
| chr3 | 147127953 | 147128847 | 895 | 1 | + | ZIC1 | NM_003412.3 |
| chr3 | 148458870 | 148459830 | 961 | 3 | + | AGTR1 | NM_004835.4 |
| chr3 | 150908512 | 150908706 | 195 | 13 | + | MED12L | NM_053002.5 |
| chr3 | 155198904 | 155200710 | 1807 | 23 | − | PLCH1 | NM_001130960.1 |
| chr3 | 157146110 | 157146277 | 168 | 5 | − | VEPH1 | NM_001167912.1 |
| chr3 | 164727066 | 164727183 | 118 | 35 | − | SI | NM_001041.3 |
| chr3 | 168838843 | 168839000 | 158 | 7 | − | MECOM | NM_004991.3 |
| chr3 | 169540059 | 169540508 | 450 | 1 | + | LRRIQ4 | NM_001080460.1 |
| chr3 | 170819255 | 170819398 | 144 | 22 | − | TNIK | NM_015028.3 |
| chr3 | 178916613 | 178916965 | 353 | 2 | + | PIK3CA | NM_006218.3 |
| chr3 | 178917477 | 178917687 | 211 | 3 | + | PIK3CA | NM_006218.3 |
| chr3 | 178919077 | 178919328 | 252 | 4 | + | PIK3CA | NM_006218.3 |
| chr3 | 178921331 | 178921577 | 247 | 5 | + | PIK3CA | NM_006218.3 |
| chr3 | 178922283 | 178922383 | 101 | 6 | + | PIK3CA | NM_006218.3 |
| chr3 | 178927382 | 178927488 | 107 | 7 | + | PIK3CA | NM_006218.3 |
| chr3 | 178927973 | 178928126 | 154 | 8 | + | PIK3CA | NM_006218.3 |
| chr3 | 178928218 | 178928353 | 136 | 9 | + | PIK3CA | NM_006218.3 |
| chr3 | 178935997 | 178936122 | 126 | 10 | + | PIK3CA | NM_006218.3 |
| chr3 | 178936974 | 178937074 | 101 | 11 | + | PIK3CA | NM_006218.3 |
| chr3 | 178937358 | 178937523 | 166 | 12 | + | PIK3CA | NM_006218.3 |
| chr3 | 178937736 | 178937840 | 105 | 13 | + | PIK3CA | NM_006218.3 |
| chr3 | 178938773 | 178938945 | 173 | 14 | + | PIK3CA | NM_006218.3 |
| chr3 | 178941868 | 178941975 | 108 | 15 | + | PIK3CA | NM_006218.3 |
| chr3 | 178942487 | 178942609 | 123 | 16 | + | PIK3CA | NM_006218.3 |
| chr3 | 178943739 | 178943839 | 101 | 17 | + | PIK3CA | NM_006218.3 |
| chr3 | 178947059 | 178947230 | 172 | 18 | + | PIK3CA | NM_006218.3 |
| chr3 | 178947791 | 178947909 | 119 | 19 | + | PIK3CA | NM_006218.3 |
| chr3 | 178948012 | 178948164 | 153 | 20 | + | PIK3CA | NM_006218.3 |
| chr3 | 178951881 | 178952152 | 272 | 21 | + | PIK3CA | NM_006218.3 |
| chr3 | 181430148 | 181431102 | 955 | 1 | + | SOX2 | NM_003106.3 |
| chr3 | 189349285 | 189349385 | 101 | 1 | + | TP63 | NM_003722.4 |
| chr3 | 189455528 | 189455657 | 130 | 2 | + | TP63 | NM_003722.4 |
| chr3 | 189456430 | 189456563 | 134 | 3 | + | TP63 | NM_003722.4 |
| chr3 | 189526060 | 189526315 | 256 | 4 | + | TP63 | NM_003722.4 |
| chr3 | 189582019 | 189582207 | 189 | 5 | + | TP63 | NM_003722.4 |
| chr3 | 189584470 | 189584586 | 117 | 6 | + | TP63 | NM_003722.4 |
| chr3 | 189585621 | 189585731 | 111 | 7 | + | TP63 | NM_003722.4 |
| chr3 | 189586368 | 189586505 | 138 | 8 | + | TP63 | NM_003722.4 |
| chr3 | 189587104 | 189587204 | 101 | 9 | + | TP63 | NM_003722.4 |
| chr3 | 189590647 | 189590784 | 138 | 10 | + | TP63 | NM_003722.4 |
| chr3 | 189604182 | 189604340 | 159 | 11 | + | TP63 | NM_003722.4 |
| chr3 | 189607128 | 189607273 | 146 | 12 | + | TP63 | NM_003722.4 |
| chr3 | 189608574 | 189608674 | 101 | 13 | + | TP63 | NM_003722.4 |
| chr3 | 189611994 | 189612291 | 298 | 14 | + | TP63 | NM_003722.4 |
| chr3 | 192516294 | 192517141 | 848 | 2 | − | MB21D2 | NM_178496.3 |
| chr4 | 1808555 | 1810599 | 2045 | 17, 18 | + | FGFR3 | NM_001163213.1 |
| chr4 | 3768690 | 3768968 | 279 | 1 | + | ADRA2C | NM_000683.3 |
| chr4 | 9783961 | 9785062 | 1102 | 1 | + | DRD5 | NM_000798.4 |
| chr4 | 20619060 | 20619186 | 127 | 36 | + | SLIT2 | NM_004787.3 |
| chr4 | 22749400 | 22749650 | 251 | 3 | + | GBA3 | NR_102355.1 |
| chr4 | 41984000 | 41984281 | 282 | 1 | + | DCAF4L1 | NM_001029955.3 |
| chr4 | 44176944 | 44177258 | 315 | 2 | − | KCTD8 | NM_198353.2 |
| chr4 | 57896425 | 57896548 | 124 | 24 | + | POLR2B | NM_000938.2 |
| chr4 | 73012693 | 73013517 | 825 | 4 | + | NPFFR2 | NM_004885.2 |
| chr4 | 94376876 | 94377125 | 250 | 11 | + | GRID2 | NM_001510.3 |
| chr4 | 110914377 | 110914477 | 101 | 19 | + | EGF | NM_001963.5 |
| chr4 | 115891585 | 115891740 | 156 | 4 | − | NDST4 | NM_022569.2 |
| chr4 | 153247160 | 153247380 | 221 | 10 | − | FBXW7 | NM_033632.3 |
| chr4 | 153249384 | 153249527 | 144 | 9 | − | FBXW7 | NM_033632.3 |
| chr4 | 162306901 | 162307560 | 660 | 16 | − | FSTL5 | NM_020116.4 |
| chr4 | 175896742 | 175898747 | 2006 | 2 | + | ADAM29 | NM_001278127.1 |
| chr4 | 187509745 | 187510374 | 630 | 27 | − | FAT1 | NM_005245.3 |
| chr4 | 187516842 | 187516980 | 139 | 26 | − | FAT1 | NM_005245.3 |
| chr4 | 187517693 | 187518325 | 633 | 25 | − | FAT1 | NM_005245.3 |
| chr4 | 187518835 | 187518946 | 112 | 24 | − | FAT1 | NM_005245.3 |
| chr4 | 187519125 | 187519279 | 155 | 23 | − | FAT1 | NM_005245.3 |
| chr4 | 187521051 | 187521514 | 464 | 22 | − | FAT1 | NM_005245.3 |
| chr4 | 187522422 | 187522580 | 159 | 21 | − | FAT1 | NM_005245.3 |
| chr4 | 187524056 | 187524188 | 133 | 20 | − | FAT1 | NM_005245.3 |
| chr4 | 187524329 | 187525131 | 803 | 19 | − | FAT1 | NM_005245.3 |
| chr4 | 187525530 | 187525728 | 199 | 18 | − | FAT1 | NM_005245.3 |
| chr4 | 187527223 | 187527367 | 145 | 17 | − | FAT1 | NM_005245.3 |
| chr4 | 187530336 | 187530474 | 139 | 16 | − | FAT1 | NM_005245.3 |
| chr4 | 187530954 | 187531169 | 216 | 15 | − | FAT1 | NM_005245.3 |
| chr4 | 187532539 | 187532929 | 391 | 14 | − | FAT1 | NM_005245.3 |
| chr4 | 187534262 | 187534496 | 235 | 13 | − | FAT1 | NM_005245.3 |
| chr4 | 187535344 | 187535498 | 155 | 12 | − | FAT1 | NM_005245.3 |
| chr4 | 187538158 | 187538355 | 198 | 11 | − | FAT1 | NM_005245.3 |
| chr4 | 187538861 | 187542929 | 4069 | 10 | − | FAT1 | NM_005245.3 |
| chr4 | 187549307 | 187549518 | 212 | 9 | − | FAT1 | NM_005245.3 |
| chr4 | 187549641 | 187549917 | 277 | 8 | − | FAT1 | NM_005245.3 |
| chr4 | 187554837 | 187554977 | 141 | 7 | − | FAT1 | NM_005245.3 |
| chr4 | 187557178 | 187557389 | 212 | 6 | − | FAT1 | NM_005245.3 |
| chr4 | 187557738 | 187558068 | 331 | 5 | − | FAT1 | NM_005245.3 |
| chr4 | 187560856 | 187560956 | 101 | 4 | − | FAT1 | NM_005245.3 |
| chr4 | 187584452 | 187584767 | 316 | 3 | − | FAT1 | NM_005245.3 |
| chr4 | 187627716 | 187630981 | 3266 | 2 | − | FAT1 | NM_005245.3 |
| chr5 | 1295105 | 1295250 | 146 | 1 | − | TERT | NM_198253.2 |
| chr5 | 11022897 | 11023091 | 195 | 17 | − | CTNND2 | NM_001332.3 |
| chr5 | 11082807 | 11082958 | 152 | 16 | − | CTNND2 | NM_001332.3 |
| chr5 | 11346483 | 11346705 | 223 | 9 | − | CTNND2 | NM_001332.3 |
| chr5 | 11364834 | 11364947 | 114 | 8 | − | CTNND2 | NM_001332.3 |
| chr5 | 11397156 | 11397256 | 101 | 6 | − | CTNND2 | NM_001332.3 |
| chr5 | 13719059 | 13719207 | 149 | 72 | − | DNAH5 | NM_001369.2 |
| chr5 | 15936690 | 15937234 | 545 | 4 | + | FBXL7 | NM_012304.4 |
| chr5 | 23522412 | 23522514 | 103 | 7 | + | PRDM9 | NM_020227.3 |
| chr5 | 26881404 | 26881715 | 312 | 12 | − | CDH9 | NM_016279.3 |
| chr5 | 26885756 | 26885968 | 213 | 11 | − | CDH9 | NM_016279.3 |
| chr5 | 26903761 | 26903931 | 171 | 6 | − | CDH9 | NM_016279.3 |
| chr5 | 41159226 | 41159326 | 101 | 12 | − | C6 | NM_000065.3 |
| chr5 | 45262057 | 45262790 | 734 | 8 | − | HCN1 | NM_021072.3 |
| chr5 | 45353201 | 45353348 | 148 | 5 | − | HCN1 | NM_021072.3 |
| chr5 | 63256300 | 63257482 | 1183 | 1 | − | HTR1A | NM_000524.3 |
| chr5 | 89943366 | 89943472 | 107 | 17 | + | GPR98 | NM_032119.3 |
| chr5 | 90040933 | 90041033 | 101 | 51 | + | GPR98 | NM_032119.3 |
| chr5 | 101834431 | 101834544 | 114 | 1 | − | SLCO6A1 | NM_173488.4 |
| chr5 | 113698588 | 113698688 | 101 | 1 | + | KCNN2 | NM_021614.3 |
| chr5 | 139192995 | 139193155 | 161 | 3 | + | PSD2 | NM_032289.2 |
| chr5 | 140165874 | 140168242 | 2369 | 1 | + | PCDHA1 | NM_031410.2 |
| chr5 | 140201500 | 140203558 | 2059 | | + | PCDHA1 | NM_031411.2 |
| chr5 | 140235927 | 140237983 | 2057 | | + | PCDHA1 | NM_031411.2 |
| chr5 | 140261864 | 140264052 | 2189 | | + | PCDHA12 | NM_018903.3 |
| chr5 | 142513531 | 142513670 | 140 | 19 | + | ARHGAP26 | NM_015071.4 |
| chr5 | 153026493 | 153026601 | 109 | 3 | + | GRIA1 | NM_001258022.1 |
| chr5 | 158139993 | 158140095 | 103 | 13 | − | EBF1 | NM_024007.4 |
| chr5 | 161524717 | 161524860 | 144 | 4 | + | GABRG2 | NM_198903.2 |
| chr5 | 176636699 | 176639007 | 2309 | 5 | + | NSD1 | NM_022455.4 |
| chr5 | 176675181 | 176675325 | 145 | 11 | + | NSD1 | NM_022455.4 |
| chr5 | 176687014 | 176687153 | 140 | 14 | + | NSD1 | NM_022455.4 |
| chr5 | 176709465 | 176709582 | 118 | 19 | + | NSD1 | NM_022455.4 |
| chr6 | 348126 | 348270 | 145 | 6 | + | DUSP22 | NM_020185.4 |
| chr6 | 26027282 | 26027418 | 137 | 1 | − | HIST1H4B | NM_003544.2 |
| chr6 | 26031884 | 26032208 | 325 | 1 | − | HIST1H3B | NM_003537.3 |
| chr6 | 26045648 | 26046020 | 373 | 1 | + | HIST1H3C | NM_003531.2 |
| chr6 | 26056116 | 26056553 | 438 | 1 | − | HIST1H1C | NM_005319.3 |
| chr6 | 26204884 | 26205157 | 274 | 1 | + | HIST1H4E | NM_003545.3 |
| chr6 | 26216704 | 26216848 | 145 | 1 | − | HIST1H2BG | NM_003518.3 |
| chr6 | 26217209 | 26217595 | 387 | 1 | + | HIST1H2AE | NM_021052.2 |
| chr6 | 26225487 | 26225675 | 189 | 1 | + | HIST1H3E | NM_003532.2 |
| chr6 | 26271336 | 26271610 | 275 | 1 | − | HIST1H3G | NM_003534.2 |
| chr6 | 27777864 | 27778028 | 165 | 1 | + | HIST1H3H | NM_003536.2 |
| chr6 | 27805924 | 27806024 | 101 | 1 | − | HIST1H2AK | NM_003510.2 |
| chr6 | 27834626 | 27835200 | 575 | 1 | − | HIST1H1B | NM_005322.2 |
| chr6 | 27839691 | 27840063 | 373 | 1 | − | HIST1H3I | NM_003533.2 |
| chr6 | 28554274 | 28554444 | 171 | 1 | − | SCAND3 | NM_052923.1 |
| chr6 | 29910586 | 29910798 | 213 | 2 | + | HLA-A | NM_002116.7 |
| chr6 | 29911084 | 29911184 | 101 | 3 | + | HLA-A | NM_002116.7 |
| chr6 | 31323135 | 31323361 | 227 | 4 | − | HLA-B | NM_005514.7 |
| chr6 | 31939824 | 31939958 | 135 | 1 | + | STK19 | NM_032454.1 |
| chr6 | 32725549 | 32725660 | 112 | | − | HLA-DQB2 | NM_001198858.1 |
| chr6 | 33282954 | 33284459 | 1506 | 2 | − | ZBTB22 | NM_001145338.1 |
| chr6 | 35773512 | 35773626 | 115 | 1 | + | LHFPL5 | NM_182548.3 |
| chr6 | 46107689 | 46108044 | 356 | 2 | + | ENPP4 | NM_014936.4 |
| chr6 | 52883079 | 52883179 | 101 | 7 | − | ICK | NM_014920.3 |
| chr6 | 55113473 | 55113573 | 101 | 3 | + | HCRTR2 | NM_001526.4 |
| chr6 | 66204658 | 66205296 | 639 | 4 | − | EYS | NM_001142800.1 |
| chr6 | 87725250 | 87726088 | 839 | 2 | + | HTR1E | NM_000865.2 |
| chr6 | 96651055 | 96652047 | 993 | 3 | + | FUT9 | NM_006581.3 |
| chr6 | 100382273 | 100382393 | 121 | 5 | − | MCHR2 | NM_032503.2 |
| chr6 | 100838250 | 100838940 | 691 | 11 | − | SIM1 | NM_005068.2 |
| chr6 | 105606462 | 105606600 | 139 | 4 | − | POPDC3 | NM_022361.4 |
| chr6 | 112671162 | 112671571 | 410 | 3 | + | RFPL4B | NM_001013734.2 |
| chr6 | 116937940 | 116938344 | 405 | 1 | + | RSPH4A | NM_001010892.2 |
| chr6 | 119337959 | 119338094 | 136 | 5 | − | FAM184A | NM_024581.5 |
| chr6 | 119341141 | 119341266 | 126 | 4 | − | FAM184A | NM_024581.5 |
| chr6 | 127796883 | 127797498 | 616 | 6 | − | SOGA3 | NM_001012279.2 |
| chr6 | 130761862 | 130762868 | 1007 | 2 | + | TMEM200A | NM_001258276.1 |
| chr6 | 134210543 | 134210975 | 433 | 1 | + | TCF21 | NM_003206.3 |
| chr6 | 146480484 | 146480608 | 125 | 3 | + | GRM1 | NM_001278065.1 |
| chr6 | 146720035 | 146720767 | 733 | 8 | + | GRM1 | NM_001278065.1 |
| chr6 | 152655142 | 152655243 | 102 | 77 | − | SYNE1 | NM_182961.3 |
| chr6 | 152763220 | 152763323 | 104 | 31 | − | SYNE1 | NM_182961.3 |
| chr6 | 158449876 | 158450036 | 161 | 3 | + | SYNJ2 | NM_003898.3 |
| chr6 | 165715111 | 165715618 | 508 | 2 | − | C6orf118 | NM_144980.3 |
| chr7 | 1586590 | 1586690 | 101 | 9 | − | TMEM184A | NM_001097620.1 |
| chr7 | 8790657 | 8791330 | 674 | 3 | + | NXPH1 | NM_152745.2 |
| chr7 | 11468613 | 11468713 | 101 | 14 | − | THSD7A | NM_015204.2 |
| chr7 | 11630120 | 11630220 | 101 | 4 | − | THSD7A | NM_015204.2 |
| chr7 | 11675890 | 11676535 | 646 | 2 | − | THSD7A | NM_015204.2 |
| chr7 | 21639523 | 21639717 | 195 | 15 | + | DNAH11 | NM_001277115.1 |
| chr7 | 34118610 | 34118795 | 186 | 13 | + | BMPER | NM_133468.4 |
| chr7 | 34125372 | 34125526 | 155 | 14 | + | BMPER | NM_133468.4 |
| chr7 | 36895203 | 36895310 | 108 | 22 | − | ELMO1 | NM_014800.10 |
| chr7 | 37955723 | 37956086 | 364 | 1 | − | SFRP4 | NM_003014.3 |
| chr7 | 37988473 | 37988621 | 149 | 2 | + | EPDR1 | NM_017549.4 |
| chr7 | 38398137 | 38398237 | 101 | | − | TRGV3 | |
| chr7 | 45614686 | 45614786 | 101 | 1 | + | ADCY1 | NM_021116.2 |
| chr7 | 50611605 | 50611705 | 101 | 2 | − | DDC | NM_000790.3 |
| chr7 | 53103444 | 53104199 | 756 | 1 | + | POM121L12 | NM_182595.3 |
| chr7 | 55086964 | 55087064 | 101 | 1 | + | EGFR | NM_005228.4 |
| chr7 | 55209978 | 55210130 | 153 | 2 | + | EGFR | NM_005228.4 |
| chr7 | 55210997 | 55211181 | 185 | 3 | + | EGFR | NM_005228.4 |
| chr7 | 55214298 | 55214433 | 136 | 4 | + | EGFR | NM_005228.4 |
| chr7 | 55218971 | 55219071 | 101 | 5 | + | EGFR | NM_005228.4 |
| chr7 | 55220238 | 55220357 | 120 | 6 | + | EGFR | NM_005228.4 |
| chr7 | 55221703 | 55221845 | 143 | 7 | + | EGFR | NM_005228.4 |
| chr7 | 55223522 | 55223639 | 118 | 8 | + | EGFR | NM_005228.4 |
| chr7 | 55224225 | 55224352 | 128 | 9 | + | EGFR | NM_005228.4 |
| chr7 | 55224438 | 55224538 | 101 | 10 | + | EGFR | NM_005228.4 |
| chr7 | 55225351 | 55225451 | 101 | 11 | + | EGFR | NM_005228.4 |
| chr7 | 55227831 | 55228031 | 201 | 12 | + | EGFR | NM_005228.4 |
| chr7 | 55229191 | 55229324 | 134 | 13 | + | EGFR | NM_005228.4 |
| chr7 | 55231421 | 55231521 | 101 | 14 | + | EGFR | NM_005228.4 |
| chr7 | 55232972 | 55233130 | 159 | 15 | + | EGFR | NM_005228.4 |
| chr7 | 55238837 | 55238937 | 101 | 16 | + | EGFR | NM_005228.4 |
| chr7 | 55240675 | 55240817 | 143 | 17 | + | EGFR | NM_005228.4 |
| chr7 | 55241613 | 55241736 | 124 | 18 | + | EGFR | NM_005228.4 |
| chr7 | 55242414 | 55242514 | 101 | 19 | + | EGFR | NM_005228.4 |
| chr7 | 55248985 | 55249171 | 187 | 20 | + | EGFR | NM_005228.4 |
| chr7 | 55259411 | 55259567 | 157 | 21 | + | EGFR | NM_005228.4 |
| chr7 | 55260446 | 55260546 | 101 | 22 | + | EGFR | NM_005228.4 |
| chr7 | 55266409 | 55266556 | 148 | 23 | + | EGFR | NM_005228.4 |
| chr7 | 55268007 | 55268107 | 101 | 24 | + | EGFR | NM_005228.4 |
| chr7 | 55268880 | 55269048 | 169 | 25 | + | EGFR | NM_005228.4 |
| chr7 | 55269401 | 55269501 | 101 | 26 | + | EGFR | NM_005228.4 |
| chr7 | 55270209 | 55270318 | 110 | 27 | + | EGFR | NM_005228.4 |
| chr7 | 55272948 | 55273310 | 363 | 28 | + | EGFR | NM_005228.4 |
| chr7 | 82581206 | 82586179 | 4974 | 5 | − | PCLO | NM_033026.5 |
| chr7 | 86394555 | 86394750 | 196 | 2 | + | GRM3 | NM_000840.2 |
| chr7 | 86415580 | 86416335 | 756 | 3 | + | GRM3 | NM_000840.2 |
| chr7 | 86493597 | 86493712 | 116 | 6 | + | GRM3 | NM_000840.2 |
| chr7 | 95157167 | 95157582 | 416 | 3 | + | ASB4 | NM_016116.2 |
| chr7 | 98256517 | 98256642 | 126 | 4 | + | NPTX2 | NM_002523.2 |
| chr7 | 104377174 | 104377335 | 162 | 2 | + | LHFPL3 | NM_199000.2 |
| chr7 | 106508168 | 106509929 | 1762 | 2 | + | PIK3CG | NM_002649.3 |
| chr7 | 111368415 | 111368577 | 163 | 52 | − | DOCK4 | NM_014705.3 |
| chr7 | 117351679 | 117351779 | 101 | 23 | − | CTTNBP2 | NM_033427.2 |
| chr7 | 119914693 | 119915730 | 1038 | 1 | + | KCND2 | NM_012281.2 |
| chr7 | 121943706 | 121944308 | 603 | 1 | − | FEZF1 | NM_001024613.3 |
| chr7 | 140453074 | 140453193 | 120 | 15 | − | BRAF | NM_004333.4 |
| chr7 | 142206549 | 142206746 | 198 | | − | TCRBV12S3 | |
| chr7 | 142498863 | 142499043 | 181 | | + | TCRVB | |
| chr7 | 146829380 | 146829579 | 200 | 8 | + | CNTNAP2 | NM_014141.5 |
| chr7 | 154561126 | 154561281 | 156 | 9 | + | DPP6 | NM_130797.3 |
| chr7 | 154862704 | 154863349 | 646 | 1 | + | HTR5A | NM_024012.3 |
| chr8 | 2820009 | 2820155 | 147 | 61 | − | CSMD1 | NM_033225.5 |
| chr8 | 4494929 | 4495029 | 101 | 2 | − | CSMD1 | NM_033225.5 |
| chr8 | 38271145 | 38271322 | 178 | 19 | − | FGFR1 | NM_001174067.1 |
| chr8 | 38271435 | 38271541 | 107 | 18 | − | FGFR1 | NM_001174067.1 |
| chr8 | 38271669 | 38271807 | 139 | 17 | − | FGFR1 | NM_001174067.1 |
| chr8 | 38272062 | 38272162 | 101 | 16 | − | FGFR1 | NM_001174067.1 |
| chr8 | 38272296 | 38272419 | 124 | 15 | − | FGFR1 | NM_001174067.1 |
| chr8 | 38273387 | 38273578 | 192 | 14 | − | FGFR1 | NM_001174067.1 |
| chr8 | 38274823 | 38274934 | 112 | 13 | − | FGFR1 | NM_001174067.1 |
| chr8 | 38275387 | 38275509 | 123 | 12 | − | FGFR1 | NM_001174067.1 |
| chr8 | 38275745 | 38275891 | 147 | 11 | − | FGFR1 | NM_001174067.1 |
| chr8 | 38277050 | 38277253 | 204 | 10 | − | FGFR1 | NM_001174067.1 |
| chr8 | 38279314 | 38279459 | 146 | 9 | − | FGFR1 | NM_001174067.1 |
| chr8 | 38282026 | 38282217 | 192 | 8 | − | FGFR1 | NM_001174067.1 |
| chr8 | 38283639 | 38283763 | 125 | 7 | − | FGFR1 | NM_001174067.1 |
| chr8 | 38285438 | 38285611 | 174 | 6 | − | FGFR1 | NM_001174067.1 |
| chr8 | 38285861 | 38285961 | 101 | 5 | − | FGFR1 | NM_001174067.1 |
| chr8 | 38287199 | 38287466 | 268 | 4 | − | FGFR1 | NM_001174067.1 |
| chr8 | 38314873 | 38315052 | 180 | 3 | − | FGFR1 | NM_001174067.1 |
| chr8 | 41166589 | 41166689 | 101 | 1 | − | SFRP1 | NM_003012.4 |
| chr8 | 55533681 | 55534104 | 424 | 2 | + | RP1 | NM_006269.1 |
| chr8 | 56015331 | 56015802 | 472 | 1 | + | XKR4 | NM_052898.1 |
| chr8 | 73479973 | 73480509 | 537 | 2 | + | KCNB2 | NM_004770.2 |
| chr8 | 73848397 | 73850116 | 1720 | 3 | + | KCNB2 | NM_004770.2 |
| chr8 | 74922235 | 74922364 | 130 | 3 | + | LY96 | NM_015364.4 |
| chr8 | 88885017 | 88886181 | 1165 | 1 | − | DCAF4L2 | NM_152418.3 |
| chr8 | 92972524 | 92972728 | 205 | 11 | − | RUNX1T1 | NM_001198634.1 |
| chr8 | 98289087 | 98290056 | 970 | 1 | − | TSPYL5 | NM_033512.2 |
| chr8 | 107782022 | 107782413 | 392 | 1 | − | ABRA | NM_139166.4 |
| chr8 | 110980373 | 110980810 | 438 | 4 | − | KCNV1 | NM_014379.3 |
| chr8 | 113256622 | 113256777 | 156 | 65 | − | CSMD3 | NM_198123.1 |
| chr8 | 113259248 | 113259360 | 113 | 64 | − | CSMD3 | NM_198123.1 |
| chr8 | 113347557 | 113347703 | 147 | 45 | − | CSMD3 | NM_198123.1 |
| chr8 | 113585729 | 113585886 | 158 | 24 | − | CSMD3 | NM_198123.1 |
| chr8 | 113988121 | 113988225 | 105 | 7 | − | CSMD3 | NM_198123.1 |
| chr8 | 128748804 | 128748904 | 101 | 1 | + | MYC | NM_002467.4 |
| chr8 | 128750493 | 128751265 | 773 | 2 | + | MYC | NM_002467.4 |
| chr8 | 128752641 | 128753204 | 564 | 3 | + | MYC | NM_002467.4 |
| chr8 | 133925337 | 133925510 | 174 | 20 | + | TG | NM_003235.4 |
| chr8 | 139163459 | 139165437 | 1979 | 13 | − | FAM135B | NM_015912.3 |
| chr8 | 139601514 | 139601679 | 166 | 65 | − | COF22A1 | NM_152888.2 |
| chr8 | 140630758 | 140631261 | 504 | 2 | − | KCNK9 | NR_104210.1 |
| chr8 | 144990492 | 144996546 | 6055 | 32 | − | PFEC | NM_201380.3 |
| chr8 | 145770918 | 145771163 | 246 | 5 | − | ARHGAP39 | NM_025251.2 |
| chr9 | 14088266 | 14088366 | 101 | 11 | − | NFIB | NM_001190737.1 |
| chr9 | 14112989 | 14113089 | 101 | 10 | − | NFIB | NM_001190737.1 |
| chr9 | 14116206 | 14116345 | 140 | 9 | − | NFIB | NM_001190737.1 |
| chr9 | 14120438 | 14120623 | 186 | 8 | − | NFIB | NM_001190737.1 |
| chr9 | 14125630 | 14125765 | 136 | 7 | − | NFIB | NM_001190737.1 |
| chr9 | 14146687 | 14146806 | 120 | 6 | − | NFIB | NM_001190737.1 |
| chr9 | 14150143 | 14150264 | 122 | 5 | − | NFIB | NM_001190737.1 |
| chr9 | 14155808 | 14155908 | 101 | 4 | − | NFIB | NM_001190737.1 |
| chr9 | 14179702 | 14179802 | 101 | 3 | − | NFIB | NM_001190737.1 |
| chr9 | 14306987 | 14307519 | 533 | 2 | − | NFIB | NM_001190737.1 |
| chr9 | 14313445 | 14313545 | 101 | 1 | − | NFIB | NM_001190737.1 |
| chr9 | 21968184 | 21968284 | 101 | 3 | − | CDKN2A | NM_058195.3 |
| chr9 | 21970899 | 21971208 | 310 | 2 | − | CDKN2A | NM_058195.3 |
| chr9 | 21974676 | 21974826 | 151 | | − | CDKN2A | NM_058195.3 |
| chr9 | 21994137 | 21994330 | 194 | 1 | − | CDKN2A | NM_058195.3 |
| chr9 | 27949107 | 27950605 | 1499 | 7 | − | LINGO2 | NM_001258282.1 |
| chr9 | 33385564 | 33385664 | 101 | 7 | − | AQP7 | NM_001170.2 |
| chr9 | 37014992 | 37015149 | 158 | 3 | − | PAX5 | NM_016734.2 |
| chr9 | 37486583 | 37486778 | 196 | 2 | + | POLR1E | NM_022490.2 |
| chr9 | 93650796 | 93650909 | 114 | 13 | + | SYK | NM_003177.6 |
| chr9 | 104499571 | 104499920 | 350 | 1 | − | GRIN3A | NM_133445.2 |
| chr9 | 111745475 | 111745575 | 101 | 6 | − | CTNNAL1 | NM_003798.3 |
| chr9 | 112898500 | 112900745 | 2246 | 8 | + | PALM2-AKAP2 | NM_007203.4 |
| chr9 | 113703900 | 113704085 | 186 | 3 | − | LPAR1 | NM_001401.3 |
| chr9 | 119976687 | 119976992 | 306 | 3 | − | ASTN2 | NM_014010.4 |
| chr9 | 121929388 | 121930287 | 900 | 8 | − | DBC1 | NM_014618.2 |
| chr9 | 131348113 | 131348216 | 104 | 19 | + | SPTAN1 | NM_001130438.2 |
| chr9 | 139390522 | 139392010 | 1489 | 34 | − | NOTCH1 | NM_017617.4 |
| chr9 | 139393349 | 139393449 | 101 | 33 | − | NOTCH1 | NM_017617.4 |
| chr9 | 139393563 | 139393711 | 149 | 32 | − | NOTCH1 | NM_017617.4 |
| chr9 | 139395003 | 139395299 | 297 | 31 | − | NOTCH1 | NM_017617.4 |
| chr9 | 139396199 | 139396365 | 167 | 30 | − | NOTCH1 | NM_017617.4 |
| chr9 | 139396446 | 139396546 | 101 | 29 | − | NOTCH1 | NM_017617.4 |
| chr9 | 139396723 | 139396940 | 218 | 28 | − | NOTCH1 | NM_017617.4 |
| chr9 | 139397633 | 139397782 | 150 | 27 | − | NOTCH1 | NM_017617.4 |
| chr9 | 139399124 | 139399556 | 433 | 26 | − | NOTCH1 | NM_017617.4 |
| chr9 | 139399761 | 139400333 | 573 | 25 | − | NOTCH1 | NM_017617.4 |
| chr9 | 139400978 | 139401091 | 114 | 24 | − | NOTCH1 | NM_017617.4 |
| chr9 | 139401167 | 139401425 | 259 | 23 | − | NOTCH1 | NM_017617.4 |
| chr9 | 139401756 | 139401889 | 134 | 22 | − | NOTCH1 | NM_017617.4 |
| chr9 | 139402406 | 139402591 | 186 | 21 | − | NOTCH1 | NM_017617.4 |
| chr9 | 139402683 | 139402837 | 155 | 20 | − | NOTCH1 | NM_017617.4 |
| chr9 | 139403321 | 139403523 | 203 | 19 | − | NOTCH1 | NM_017617.4 |
| chr9 | 139404184 | 139404413 | 230 | 18 | − | NOTCH1 | NM_017617.4 |
| chr9 | 139405104 | 139405257 | 154 | 17 | − | NOTCH1 | NM_017617.4 |
| chr9 | 139405603 | 139405723 | 121 | 16 | − | NOTCH1 | NM_017617.4 |
| chr9 | 139407472 | 139407586 | 115 | 15 | − | NOTCH1 | NM_017617.4 |
| chr9 | 139407843 | 139407989 | 147 | 14 | − | NOTCH1 | NM_017617.4 |
| chr9 | 139408961 | 139409154 | 194 | 13 | − | NOTCH1 | NM_017617.4 |
| chr9 | 139409741 | 139409852 | 112 | 12 | − | NOTCH1 | NM_017617.4 |
| chr9 | 139409934 | 139410168 | 235 | 11 | − | NOTCH1 | NM_017617.4 |
| chr9 | 139410432 | 139410546 | 115 | 10 | − | NOTCH1 | NM_017617.4 |
| chr9 | 139411723 | 139411837 | 115 | 9 | − | NOTCH1 | NM_017617.4 |
| chr9 | 139412203 | 139412389 | 187 | 8 | − | NOTCH1 | NM_017617.4 |
| chr9 | 139412588 | 139412744 | 157 | 7 | − | NOTCH1 | NM_017617.4 |
| chr9 | 139413042 | 139413276 | 235 | 6 | − | NOTCH1 | NM_017617.4 |
| chr9 | 139413894 | 139414017 | 124 | 5 | − | NOTCH1 | NM_017617.4 |
| chr9 | 139417301 | 139417640 | 340 | 4 | − | NOTCH1 | NM_017617.4 |
| chr9 | 139418168 | 139418431 | 264 | 3 | − | NOTCH1 | NM_017617.4 |
| chr9 | 139438465 | 139438565 | 101 | 2 | − | NOTCH1 | NM_017617.4 |
| chr9 | 139440158 | 139440258 | 101 | 1 | − | NOTCH1 | NM_017617.4 |
| chr10 | 7214456 | 7214623 | 168 | 18 | − | SFMBT2 | NM_001018039.1 |
| chr10 | 18276392 | 18276492 | 101 | 7 | + | SLC39A12 | NM_001145195.1 |
| chr10 | 43882405 | 43882523 | 119 | 4 | − | HNRNPF | NM_001098206.1 |
| chr10 | 45941018 | 45941118 | 101 | 14 | + | ALOX5 | NM_000698.4 |
| chr10 | 50819199 | 50820314 | 1116 | 1 | + | SLC18A3 | NM_003055.2 |
| chr10 | 55955479 | 55955595 | 117 | 12 | − | PCDH15 | NM_001142763.1 |
| chr10 | 56138541 | 56138702 | 162 | 5 | − | PCDH15 | NM_001142763.1 |
| chr10 | 62647976 | 62648791 | 816 | 6 | − | RHOBTB1 | NM_014836.4 |
| chr10 | 68686714 | 68688016 | 1303 | | − | CTNNA3 | NM_013266.3 |
| chr10 | 81070746 | 81070846 | 101 | 24 | + | ZMIZ1 | NM_020338.3 |
| chr10 | 84745206 | 84745340 | 135 | 9 | + | NRG3 | NM_001010848.3 |
| chr10 | 87614252 | 87614372 | 121 | 8 | − | GRID1 | NM_017551.2 |
| chr10 | 89624216 | 89624316 | 101 | 1 | + | PTEN | NM_000314.6 |
| chr10 | 89653774 | 89653874 | 101 | 2 | + | PTEN | NM_000314.6 |
| chr10 | 89685242 | 89685342 | 101 | 3 | + | PTEN | NM_000314.6 |
| chr10 | 89690774 | 89690874 | 101 | 4 | + | PTEN | NM_000314.6 |
| chr10 | 89692769 | 89693008 | 240 | 5 | + | PTEN | NM_000314.6 |
| chr10 | 89711874 | 89712016 | 143 | 6 | + | PTEN | NM_000314.6 |
| chr10 | 89717609 | 89717776 | 168 | 7 | + | PTEN | NM_000314.6 |
| chr10 | 89720650 | 89720875 | 226 | 8 | + | PTEN | NM_000314.6 |
| chr10 | 89725043 | 89725229 | 187 | 9 | + | PTEN | NM_000314.6 |
| chr10 | 117884807 | 117885065 | 259 | 6 | − | GFRA1 | NM_005264.5 |
| chr11 | 532635 | 532755 | 121 | 5 | − | HRAS | NM_001130442.2 |
| chr11 | 533296 | 533612 | 317 | 4 | − | HRAS | NM_001130442.2 |
| chr11 | 533766 | 533945 | 180 | 3 | − | HRAS | NM_001130442.2 |
| chr11 | 534211 | 534322 | 112 | 2 | − | HRAS | NM_001130442.2 |
| chr11 | 5529367 | 5530481 | 1115 | 2 | − | UBQLN3 | NM_017481.3 |
| chr11 | 21592314 | 21592463 | 150 | 18 | + | NELL1 | NM_006157.4 |
| chr11 | 66617733 | 66617886 | 154 | 17 | − | PC | NM_022172.2 |
| chr11 | 67209118 | 67209218 | 101 | 4 | − | CORO1B | NM_020441.2 |
| chr11 | 68696699 | 68696799 | 101 | 8 | + | IGHMBP2 | NM_002180.2 |
| chr11 | 69456081 | 69456279 | 199 | 1 | + | CCND1 | NM_053056.2 |
| chr11 | 69457798 | 69458014 | 217 | 2 | + | CCND1 | NM_053056.2 |
| chr11 | 69458599 | 69458759 | 161 | 3 | + | CCND1 | NM_053056.2 |
| chr11 | 69462761 | 69462910 | 150 | 4 | + | CCND1 | NM_053056.2 |
| chr11 | 69465885 | 69466050 | 166 | 5 | + | CCND1 | NM_053056.2 |
| chr11 | 70049565 | 70049851 | 287 | 1 | + | FADD | NM_003824.3 |
| chr11 | 70052238 | 70052579 | 342 | 2 | + | FADD | NM_003824.3 |
| chr11 | 70253397 | 70253497 | 101 | 3 | + | CTTN | NM_001184740.1 |
| chr11 | 70253610 | 70253710 | 101 | 4 | + | CTTN | NM_001184740.1 |
| chr11 | 70255936 | 70256066 | 131 | 5 | + | CTTN | NM_001184740.1 |
| chr11 | 70260647 | 70260758 | 112 | 6 | + | CTTN | NM_001184740.1 |
| chr11 | 70261746 | 70261846 | 101 | 7 | + | CTTN | NM_001184740.1 |
| chr11 | 70263118 | 70263229 | 112 | 8 | + | CTTN | NM_001184740.1 |
| chr11 | 70265851 | 70265962 | 112 | 9 | + | CTTN | NM_001184740.1 |
| chr11 | 70266505 | 70266616 | 112 | 10 | + | CTTN | NM_001184740.1 |
| chr11 | 70269023 | 70269123 | 101 | 11 | + | CTTN | NM_001184740.1 |
| chr11 | 70271422 | 70271522 | 101 | 12 | + | CTTN | NM_001184740.1 |
| chr11 | 70275156 | 70275305 | 150 | 13 | + | CTTN | NM_001184740.1 |
| chr11 | 70277291 | 70277391 | 101 | 14 | + | CTTN | NM_001184740.1 |
| chr11 | 70279206 | 70279384 | 179 | 15 | + | CTTN | NM_001184740.1 |
| chr11 | 70279738 | 70279838 | 101 | 16 | + | CTTN | NM_001184740.1 |
| chr11 | 70281128 | 70281228 | 101 | 17 | + | CTTN | NM_001184740.1 |
| chr11 | 70281571 | 70281850 | 280 | 18 | + | CTTN | NM_001184740.1 |
| chr11 | 70282387 | 70282514 | 128 | 19 | + | CTTN | NM_001184740.1 |
| chr11 | 84027931 | 84028145 | 215 | | − | DLG2 | NM_001142699.1 |
| chr11 | 101981579 | 101981900 | 322 | 1 | + | YAP1 | NM_001130145.2 |
| chr11 | 101984874 | 101985125 | 252 | 2 | + | YAP1 | NM_001130145.2 |
| chr11 | 102033186 | 102033302 | 117 | 3 | + | YAP1 | NM_001130145.2 |
| chr11 | 102056748 | 102056862 | 115 | 4 | + | YAP1 | NM_001130145.2 |
| chr11 | 102076623 | 102076805 | 183 | 5 | + | YAP1 | NM_001130145.2 |
| chr11 | 102080221 | 102080321 | 101 | 6 | + | YAP1 | NM_001130145.2 |
| chr11 | 102094352 | 102094483 | 132 | 7 | + | YAP1 | NM_001130145.2 |
| chr11 | 102098199 | 102098312 | 114 | 8 | + | YAP1 | NM_001130145.2 |
| chr11 | 102100432 | 102100671 | 240 | 9 | + | YAP1 | NM_001130145.2 |
| chr11 | 102738638 | 102738799 | 162 | 5, 6 | − | MMP12 | NM_002426.5 |
| chr11 | 122848357 | 122848580 | 224 | 3 | − | BSX | NM_001098169.1 |
| chr11 | 132016210 | 132016342 | 133 | 2 | + | NTM | NM_001144058.1 |
| chr11 | 132527002 | 132527155 | 154 | 2 | − | OPCML | NM_002545.4 |
| chr12 | 4479555 | 4479838 | 284 | 3 | − | FGF23 | NM_020638.2 |
| chr12 | 7635997 | 7636248 | 252 | 12 | − | CD163 | NM_004244.5 |
| chr12 | 20832976 | 20833115 | 140 | 16 | + | PDE3A | NM_000921.4 |
| chr12 | 24398208 | 24398319 | 112 | | − | SOX5 | NM_152989.4 |
| chr12 | 25360174 | 25360274 | 101 | 6 | − | KRAS | NM_033360.3 |
| chr12 | 25362729 | 25362846 | 118 | 6 | − | KRAS | NM_033360.3 |
| chr12 | 25368375 | 25368495 | 121 | 5 | − | KRAS | NM_033360.3 |
| chr12 | 25378548 | 25378707 | 160 | 4 | − | KRAS | NM_033360.3 |
| chr12 | 41966189 | 41967569 | 1381 | 10 | + | PDZRN4 | NM_001164595.1 |
| chr12 | 48526695 | 48526800 | 106 | 7 | + | PFKM | NM_001166686.1 |
| chr12 | 50367072 | 50367303 | 232 | 1 | + | AQP6 | NM_001652.3 |
| chr12 | 54396247 | 54396387 | 141 | 2 | + | HOXC9 | NM_006897.2 |
| chr12 | 56221127 | 56222202 | 1076 | 2 | − | DNAJC14 | NM_032364.5 |
| chr12 | 63541188 | 63541364 | 177 | 2 | − | AVPR1A | NM_000706.4 |
| chr12 | 69202214 | 69202314 | 101 | 1 | + | MDM2 | NM_002392.5 |
| chr12 | 69202980 | 69203080 | 101 | 2 | + | MDM2 | NM_002392.5 |
| chr12 | 69207321 | 69207421 | 101 | 3 | + | MDM2 | NM_002392.5 |
| chr12 | 69210591 | 69210725 | 135 | 4 | + | MDM2 | NM_002392.5 |
| chr12 | 69214079 | 69214179 | 101 | 5 | + | MDM2 | NM_002392.5 |
| chr12 | 69218126 | 69218226 | 101 | 6 | + | MDM2 | NM_002392.5 |
| chr12 | 69218333 | 69218433 | 101 | 7 | + | MDM2 | NM_002392.5 |
| chr12 | 69222550 | 69222711 | 162 | 8 | + | MDM2 | NM_002392.5 |
| chr12 | 69229608 | 69229764 | 157 | 9 | + | MDM2 | NM_002392.5 |
| chr12 | 69230440 | 69230540 | 101 | 10 | + | MDM2 | NM_002392.5 |
| chr12 | 69233053 | 69233629 | 577 | 11 | + | MDM2 | NM_002392.5 |
| chr12 | 70003992 | 70004471 | 480 | 1 | − | LRRC10 | NM_201550.3 |
| chr12 | 72056923 | 72057344 | 422 | 1 | − | ZFC3H1 | NM_144982.4 |
| chr12 | 78400201 | 78400964 | 764 | 8 | + | NAV3 | NM_014903.5 |
| chr12 | 81471975 | 81472120 | 146 | 1 | + | ACSS3 | NM_024560.3 |
| chr12 | 109972412 | 109972571 | 160 | 28 | + | UBE3B | NM_183415.2 |
| chr12 | 112182634 | 112182734 | 101 | 14 | + | ACAD10 | NM_001136538.1 |
| chr12 | 113515302 | 113515402 | 101 | 2 | + | DTX1 | NM_004416.2 |
| chr12 | 113704009 | 113704109 | 101 | 5 | + | TPCN1 | NM_001143819.2 |
| chr12 | 122812641 | 122812741 | 101 | 17 | − | CLIP1 | NM_001247997.1 |
| chr12 | 130184385 | 130185232 | 848 | 2 | − | TMEM132D | NM_133448.2 |
| chr13 | 36700036 | 36700223 | 188 | 2 | − | DCLK1 | NM_004734.4 |
| chr13 | 67799495 | 67802562 | 3068 | 2 | − | PCDH9 | NM_203487.2 |
| chr13 | 70549770 | 70549923 | 154 | 2 | − | KLHL1 | NM_020866.2 |
| chr13 | 88327662 | 88329861 | 2200 | 2 | + | SLITRK5 | NM_015567.1 |
| chr13 | 108518048 | 108518794 | 747 | 1 | − | FAM155A | NM_001080396.2 |
| chr14 | 23346341 | 23346654 | 314 | 7 | + | LRP10 | NM_014045.4 |
| chr14 | 23444182 | 23444313 | 132 | 5 | − | AJUBA | NM_032876.5 |
| chr14 | 23447552 | 23447654 | 103 | 2 | − | AJUBA | NM_032876.5 |
| chr14 | 23450499 | 23451384 | 886 | 1 | − | AJUBA | NM_032876.5 |
| chr14 | 23887494 | 23887594 | 101 | 30 | − | MYH7 | NM_000257.3 |
| chr14 | 24788947 | 24789094 | 148 | 22 | − | ADCY4 | NM_001198592.1 |
| chr14 | 30046451 | 30046551 | 101 | 18 | − | PRKD1 | NM_002742.2 |
| chr14 | 42360586 | 42361026 | 441 | 4 | + | LRFN5 | NM_152447.4 |
| chr14 | 47530495 | 47530773 | 279 | 7 | − | MDGA2 | NM_001113498.2 |
| chr14 | 52520338 | 52520463 | 126 | 5 | − | NID2 | NM_007361.3 |
| chr14 | 59112195 | 59113679 | 1485 | 4 | + | DACT1 | NM_016651.5 |
| chr14 | 70633590 | 70634947 | 1358 | 2 | − | SLC8A3 | NM_183002.2 |
| chr14 | 80328090 | 80328224 | 135 | 17 | + | NRXN3 | NM_004796.5 |
| chr14 | 95921754 | 95922001 | 248 | 5 | − | SYNE3 | NM_152592.4 |
| chr14 | 102508971 | 102509085 | 115 | 69 | + | DYNC1H1 | NM_001376.4 |
| chr14 | 105246425 | 105246553 | 129 | 3 | − | AKT1 | NM_001014431.1 |
| chr14 | 106054571 | 106054674 | 104 | | − | DKFZp686O16217 | |
| chr15 | 23811018 | 23812439 | 1422 | 1 | + | MKRN3 | NM_005664.3 |
| chr15 | 24921050 | 24924416 | 3367 | 1 | + | NPAP1 | NM_018958.2 |
| chr15 | 26806093 | 26806284 | 192 | 8 | − | GABRB3 | NM_000814.5 |
| chr15 | 28326815 | 28326986 | 172 | 2 | − | OCA2 | NM_000275.2 |
| chr15 | 45007630 | 45007843 | 214 | 2 | + | B2M | NM_004048.2 |
| chr15 | 48500014 | 48500322 | 309 | 2 | + | SLC12A1 | NM_001184832.1 |
| chr15 | 74883637 | 74883737 | 101 | 6 | + | ARID3B | NM_006465.3 |
| chr15 | 75641257 | 75641451 | 195 | 2 | + | NEIL1 | NM_001256552.1 |
| chr15 | 79292108 | 79292224 | 117 | 18 | − | RASGRF1 | NM_002891.4 |
| chr15 | 84581961 | 84582061 | 101 | 16 | + | ADAMTSL3 | NM_207517.2 |
| chr15 | 89424689 | 89424837 | 149 | 3 | − | HAPFN3 | NM_178232.3 |
| chr15 | 91835658 | 91835758 | 101 | 14 | + | SV2B | NM_014848.6 |
| chr15 | 96875607 | 96875744 | 138 | 1 | + | NR2F2 | NM_021005.3 |
| chr16 | 3293433 | 3293684 | 252 | 10 | − | MEFV | NM_000243.2 |
| chr16 | 3452110 | 3452372 | 263 | 1 | + | ZNF174 | NM_003450.2 |
| chr16 | 3788559 | 3788673 | 115 | 26 | − | CREBBP | NM_004380.2 |
| chr16 | 50811735 | 50811852 | 118 | 7 | + | CYLD | NM_001042355.1 |
| chr16 | 56973961 | 56974110 | 150 | 6 | + | HERPUD1 | NM_014685.3 |
| chr16 | 64984669 | 64984857 | 189 | 12 | − | CDH11 | NM_001797.3 |
| chr16 | 65032503 | 65032725 | 223 | 4 | − | CDH11 | NM_001797.3 |
| chr16 | 67650647 | 67650781 | 135 | 5 | + | CTCF | NM_006565.3 |
| chr16 | 72188111 | 72188258 | 148 | 4 | − | PMFBP1 | NM_031293.2 |
| chr16 | 77465304 | 77465454 | 151 | 3 | − | ADAMTS18 | NM_199355.3 |
| chr16 | 84132677 | 84132849 | 173 | 3 | − | MBTPS1 | NM_003791.3 |
| chr16 | 89986155 | 89986384 | 230 | 1 | + | MC1R | NM_002386.3 |
| chr16 | 90161901 | 90162305 | 405 | | + | TUBB4Q | |
| chr17 | 7572918 | 7573018 | 101 | 11 | − | TP53 | NM_001276760.1 |
| chr17 | 7573926 | 7574033 | 108 | 10 | − | TP53 | NM_001276760.1 |
| chr17 | 7576525 | 7576658 | 134 | | − | TP53 | NM_001276760.1 |
| chr17 | 7576839 | 7576939 | 101 | 9 | − | TP53 | NM_001276760.1 |
| chr17 | 7577018 | 7577155 | 138 | 8 | − | TP53 | NM_001276760.1 |
| chr17 | 7577498 | 7577608 | 111 | 7 | − | TP53 | NM_001276760.1 |
| chr17 | 7578176 | 7578289 | 114 | 6 | − | TP53 | NM_001276760.1 |
| chr17 | 7578369 | 7578554 | 186 | 5 | − | TP53 | NM_001276760.1 |
| chr17 | 7579310 | 7579580 | 271 | 4 | − | TP53 | NM_001276760.1 |
| chr17 | 7579660 | 7579760 | 101 | 3 | − | TP53 | NM_001276760.1 |
| chr17 | 7579826 | 7579926 | 101 | 2 | − | TP53 | NM_001276760.1 |
| chr17 | 10303757 | 10304049 | 293 | 27 | − | MYH8 | NM_002472.2 |
| chr17 | 10369589 | 10369733 | 145 | 4 | − | MYH4 | NM_017533.2 |
| chr17 | 21318730 | 21319867 | 1138 | | + | KCNJ12 | NM_001194958.2 |
| chr17 | 26684313 | 26684473 | 161 | 1, 2 | − | POLDIP2 | NM_015584.4 |
| chr17 | 34077206 | 34077306 | 101 | 2 | − | GAS2L2 | NM_139285.3 |
| chr17 | 37855776 | 37855876 | 101 | | + | ERBB2 | NM_001005862.2 |
| chr17 | 37856478 | 37856578 | 101 | 1 | + | ERBB2 | NM_004448.3 |
| chr17 | 37863232 | 37863452 | 221 | 2 | + | ERBB2 | NM_004448.3 |
| chr17 | 37864563 | 37864797 | 235 | 3 | + | ERBB2 | NM_004448.3 |
| chr17 | 37865560 | 37865715 | 156 | 4 | + | ERBB2 | NM_004448.3 |
| chr17 | 37866050 | 37866150 | 101 | 5 | + | ERBB2 | NM_004448.3 |
| chr17 | 37866328 | 37866464 | 137 | 6 | + | ERBB2 | NM_004448.3 |
| chr17 | 37866582 | 37866744 | 163 | 7 | + | ERBB2 | NM_004448.3 |
| chr17 | 37868170 | 37868310 | 141 | 8 | + | ERBB2 | NM_004448.3 |
| chr17 | 37868564 | 37868711 | 148 | 9 | + | ERBB2 | NM_004448.3 |
| chr17 | 37869395 | 37869532 | 138 | | + | ERBB2 | NM_004448.3 |
| chr17 | 37871525 | 37871625 | 101 | 10 | + | ERBB2 | NM_004448.3 |
| chr17 | 37871688 | 37871799 | 112 | 11 | + | ERBB2 | NM_004448.3 |
| chr17 | 37871982 | 37872202 | 221 | 12 | + | ERBB2 | NM_004448.3 |
| chr17 | 37872543 | 37872696 | 154 | 13 | + | ERBB2 | NM_004448.3 |
| chr17 | 37872757 | 37872868 | 112 | 14 | + | ERBB2 | NM_004448.3 |
| chr17 | 37873562 | 37873747 | 186 | 15 | + | ERBB2 | NM_004448.3 |
| chr17 | 37876013 | 37876113 | 101 | 16 | + | ERBB2 | NM_004448.3 |
| chr17 | 37879561 | 37879720 | 160 | 17 | + | ERBB2 | NM_004448.3 |
| chr17 | 37879780 | 37879923 | 144 | 18 | + | ERBB2 | NM_004448.3 |
| chr17 | 37880154 | 37880273 | 120 | 19 | + | ERBB2 | NM_004448.3 |
| chr17 | 37880968 | 37881174 | 207 | 20 | + | ERBB2 | NM_004448.3 |
| chr17 | 37881291 | 37881467 | 177 | 21 | + | ERBB2 | NM_004448.3 |
| chr17 | 37881567 | 37881667 | 101 | 22 | + | ERBB2 | NM_004448.3 |
| chr17 | 37881949 | 37882116 | 168 | 23 | + | ERBB2 | NM_004448.3 |
| chr17 | 37882804 | 37882922 | 119 | 24 | + | ERBB2 | NM_004448.3 |
| chr17 | 37883057 | 37883266 | 210 | 25 | + | ERBB2 | NM_004448.3 |
| chr17 | 37883537 | 37883810 | 274 | 26 | + | ERBB2 | NM_004448.3 |
| chr17 | 37883931 | 37884307 | 377 | 27 | + | ERBB2 | NM_004448.3 |
| chr17 | 42248166 | 42248420 | 255 | 1 | + | ASB16 | NM_080863.4 |
| chr17 | 65026607 | 65026939 | 333 | 4 | + | CACNG4 | NM_014405.3 |
| chr17 | 80788943 | 80790329 | 1387 | | + | TBCD | NM_005993.4 |
| chr18 | 580456 | 580887 | 432 | 1 | + | CETN1 | NM_004066.2 |
| chr18 | 5397092 | 5397423 | 332 | 18 | − | EPB41L3 | NM_012307.3 |
| chr18 | 5415858 | 5416180 | 323 | 13 | − | EPB41L3 | NM_012307.3 |
| chr18 | 13825967 | 13826657 | 691 | 1 | + | MC5R | NM_005913.2 |
| chr18 | 13884632 | 13885468 | 837 | 2 | − | MC2R | NM_000529.2 |
| chr18 | 22804523 | 22807584 | 3062 | 4 | − | ZNF521 | NM_015461.2 |
| chr18 | 63547682 | 63547974 | 293 | 12 | + | CDH7 | NM_033646.2 |
| chr18 | 64172066 | 64172442 | 377 | 12 | − | CDH19 | NM_021153.3 |
| chr18 | 67406200 | 67406339 | 140 | 6 | + | DOK6 | NM_152721.5 |
| chr19 | 2121161 | 2121310 | 150 | 13 | − | AP3D1 | NM_001261826.1 |
| chr19 | 3964691 | 3964914 | 224 | 3 | − | DAPK3 | NM_001348.2 |
| chr19 | 5455842 | 5456254 | 413 | 1 | + | ZNRF4 | NM_181710.3 |
| chr19 | 10597317 | 10597504 | 188 | 6 | − | KEAP1 | NM_203500.1 |
| chr19 | 10599857 | 10600054 | 198 | 5 | − | KEAP1 | NM_203500.1 |
| chr19 | 10600313 | 10600539 | 227 | 4 | − | KEAP1 | NM_203500.1 |
| chr19 | 10602242 | 10602948 | 707 | 3 | − | KEAP1 | NM_203500.1 |
| chr19 | 10610060 | 10610719 | 660 | 2 | − | KEAP1 | NM_203500.1 |
| chr19 | 20728545 | 20728771 | 227 | 4 | − | ZNF737 | NM_001159293.1 |
| chr19 | 36218401 | 36218501 | 101 | 16 | + | KMT2B | NM_014727.2 |
| chr19 | 37440566 | 37440780 | 215 | 7 | + | ZNF568 | NM_198539.3 |
| chr19 | 42260627 | 42260788 | 162 | 2 | + | CEACAM6 | NM_002483.6 |
| chr19 | 49933848 | 49933948 | 101 | 12 | − | SLC17A7 | NM_020309.3 |
| chr19 | 52619657 | 52620045 | 389 | 4 | − | ZNF616 | NM_178523.4 |
| chr19 | 54313207 | 54314440 | 1234 | 3 | − | NLRP12 | NM_001277126.1 |
| chr19 | 54466452 | 54466611 | 160 | 1 | + | CACNG8 | NM_031895.5 |
| chr19 | 54677829 | 54678114 | 286 | 8 | − | MBOAT7 | NM_024298.4 |
| chr19 | 55377999 | 55378186 | 188 | 9 | + | KIR3DL2 | NM_006737.3 |
| chr19 | 57640090 | 57642406 | 2317 | 4 | + | USP29 | NM_020903.2 |
| chr20 | 29625872 | 29625984 | 113 | 4 | + | FRG1B | NR_003579.1 |
| chr20 | 32264537 | 32264785 | 249 | 7 | − | E2F1 | NM_005225.2 |
| chr20 | 32264910 | 32265136 | 227 | 6 | − | E2F1 | NM_005225.2 |
| chr20 | 32265231 | 32265346 | 116 | 5 | − | E2F1 | NM_005225.2 |
| chr20 | 32266006 | 32266159 | 154 | 4 | − | E2F1 | NM_005225.2 |
| chr20 | 32267560 | 32267780 | 221 | 3 | − | E2F1 | NM_005225.2 |
| chr20 | 32268127 | 32268227 | 101 | 2 | − | E2F1 | NM_005225.2 |
| chr20 | 32273809 | 32274070 | 262 | 1 | − | E2F1 | NM_005225.2 |
| chr20 | 33033101 | 33033228 | 128 | 12 | + | ITCH | NM_001257137.2 |
| chr20 | 36850850 | 36850999 | 150 | 10 | − | KIAA1755 | NM_001029864.1 |
| chr20 | 50139651 | 50140541 | 891 | 2 | − | NFATC2 | NM_173091.3 |
| chr20 | 61488772 | 61488905 | 134 | 4 | − | TCFL5 | NM_006602.3 |
| chr21 | 41142929 | 41143079 | 151 | 4 | + | IGSF5 | NM_001080444.1 |
| chr21 | 47545907 | 47546046 | 140 | 26 | + | COL6A2 | NM_001849.3 |
| chr22 | 22127161 | 22127271 | 111 | 7 | − | MAPK1 | NM_002745.4 |
| chr22 | 30722668 | 30722864 | 197 | 1 | − | TBC1D10A | NM_001204240.1 |
| chr22 | 32554979 | 32555104 | 126 | 1 | − | C22orf42 | NM_001010859.1 |
| chr22 | 36708122 | 36708258 | 137 | 14 | − | MYH9 | NM_002473.5 |
| chr22 | 37603210 | 37603433 | 224 | 2 | − | SSTR3 | NM_001051.4 |
| chr22 | 41565506 | 41565620 | 115 | 26 | + | EP300 | NM_001429.3 |
| chr22 | 42070953 | 42071075 | 123 | 3 | − | NHP2L1 | NM_001003796.1 |
| chr22 | 42538728 | 42538881 | 154 | 3 | − | CYP2D7P1 | NR_002570.3 |
| chrX | 12734345 | 12734914 | 570 | 15 | + | FRMPD4 | NM_014728.3 |
| chrX | 30268766 | 30269598 | 833 | 2 | + | MAGEB1 | NM_177404.2 |
| chrX | 32328251 | 32328385 | 135 | 42 | − | DMD | NM_004006.2 |
| chrX | 34148022 | 34150317 | 2296 | 1 | − | FAM47A | NM_203408.3 |
| chrX | 41000545 | 41000684 | 140 | 9 | + | USP9X | NM_001039590.2 |
| chrX | 53560971 | 53561071 | 101 | 83 | − | HUWE1 | NM_031407.6 |
| chrX | 74494188 | 74494382 | 195 | 1 | + | UPRT | NM_145052.3 |
| chrX | 78616825 | 78616976 | 152 | 5 | − | ITM2A | NM_004867.4 |
| chrX | 79281104 | 79281236 | 133 | 4 | + | TBX22 | NM_016954.2 |
| chrX | 92926977 | 92928298 | 1322 | 1 | − | NAP1L3 | NM_004538.5 |
| chrX | 102337167 | 102337278 | 112 | 9 | − | NXF3 | NM_022052.1 |
| chrX | 107976933 | 107979425 | 2493 | 1 | − | IRS4 | NM_003604.2 |
| chrX | 110644216 | 110644425 | 210 | 3 | − | DCX | NM_000555.3 |
| chrX | 114540769 | 114540930 | 162 | 4 | + | LUZP4 | NM_016383.4 |
| chrX | 123514614 | 123515060 | 447 | 32 | − | TENM1 | NM_001163278.1 |
| chrX | 134483098 | 134483239 | 142 | 3 | + | ZNF449 | NM_152695.5 |
| chrX | 142716465 | 142718808 | 2344 | 2 | − | SLITRK4 | NM_001184749.2 |
| chrX | 142795425 | 142795525 | 101 | 2 | − | SPANXN2 | NM_001009615.2 |

Plasma and PBL samples from HNSCC patients at diagnosis and healthy donors by CAPP-Seq, utilizing 10-30 ng of input DNA were profiled. To achieve sensitive detection of ctDNA at low abundance, we applied a CAPP-Seq selector optimized to maximize the number of detected mutations in HNSCC (Table 2 and FIG. 10). We further improved our analytical sensitivity through integrated Digital Error Suppression (iDES), incorporating custom molecular barcodes and removing background base substitution errors as identified within healthy donor plasma samples (Methods).

TABLE 2
Reported yields of cell-free DNA
normalized to total plasma volume

| sampleID | dsDNApermLPlasma | timepoint |
|---|---|---|
| 1 | 10.69473684 | Normal |
| 2 | 19.6137931 | Normal |
| 3 | 11.2 | Normal |
| 4 | 9.76 | Normal |
| 5 | 11.57 | Normal |
| 6 | 7.72 | Normal |
| 7 | 15.83283582 | Normal |
| 1 | 5.09 | Diagnosis |
| 1 | 9.6 | Post-surgery |
| 2 | 12.34 | Diagnosis |
| 2 | 16.65 | Mid-radiotherapy |
| 3 | 5.55 | Diagnosis |
| 3 | 6.443076923 | Mid-radiotherapy |
| 3 | 5.659701493 | Post-treatment-1 |
| 3 | 8.516129032 | Post-treatment-2 |
| 4 | 13.65 | Diagnosis |
| 4 | 13.18 | Mid-radiotherapy |
| 5 | 11.76 | Diagnosis |
| 5 | 8.66 | Mid-radiotherapy |
| 6 | 6.75 | Diagnosis |
| 6 | 9.6 | Mid-radiotherapy |
| 6 | 13.23 | Post-treatment-1 |
| 6 | 8.68 | Post-treatment-2 |
| 7 | 10.28571429 | Diagnosis |
| 7 | 15.08571429 | Mid-radiotherapy |
| 7 | 4.96875 | Post-surgery |
| 7 | 6.941538462 | Post-treatment-1 |
| 8 | 16.68 | Diagnosis |
| 8 | 12.93 | Mid-radiotherapy |
| 8 | 23.21 | Post-surgery |
| 9 | 20.01509434 | Diagnosis |
| 9 | 20.05970149 | Mid-radiotherapy |
| 10 | 12.18 | Diagnosis |
| 10 | 14.32 | Mid-radiotherapy |
| 10 | 8.93 | Post-surgery |
| 11 | 27.04 | Diagnosis |
| 11 | 20.06 | Mid-radiotherapy |
| 11 | 26.68 | Post-surgery |
| 11 | 9.07 | Post-treatment-1 |
| 12 | 7.2 | Diagnosis |
| 12 | 6.93 | Post-surgery |
| 13 | 8.87 | Diagnosis |
| 13 | 7.69 | Post-surgery |
| 14 | 5.73 | Diagnosis |
| 14 | 9.28 | Post-surgery |
| 15 | 17.31940299 | Diagnosis |
| 15 | 19.63636364 | Mid-radiotherapy |
| 16 | 21.75 | Diagnosis |
| 16 | 30.28 | Post-surgery |
| 17 | 14.02 | Diagnosis |
| 17 | 15.65 | Post-surgery |
| 18 | 8.076 | Diagnosis |
| 18 | 8.671 | Mid-radiotherapy |
| 18 | 7.504 | Post-surgery |
| 18 | 10.386 | Post-treatment-1 |
| 19 | 5.16 | Diagnosis |
| 19 | 11.41333333 | Mid-radiotherapy |
| 19 | 17.6 | Post-surgery |
| 20 | 52.58181818 | Diagnosis |
| 20 | 9.523809524 | Mid-radiotherapy |
| 20 | 24.38709677 | Post-surgery |
| 20 | 55.8 | Post-treatment-1 |
| 21 | 8.903225806 | Diagnosis |
| 21 | 10.28571429 | Mid-radiotherapy |
| 21 | 14.55 | Post-surgery |
| 21 | 9.68 | Post-treatment-1 |
| 22 | 69.96 | Diagnosis |
| 22 | 10.25 | Mid-radiotherapy |
| 22 | 26.71 | Post-treatment-1 |
| 23 | 8.023880597 | Diagnosis |
| 23 | 6.889655172 | Mid-radiotherapy |
| 23 | 13.73333333 | Post-surgery |
| 24 | 4.34 | Diagnosis |
| 24 | 11.78 | Post-surgery |
| 25 | 13.76 | Diagnosis |
| 25 | 10 | Post-surgery |
| 26 | 31.16 | Diagnosis |
| 26 | 24 | Mid-radiotherapy |
| 26 | 16.8 | Post-treatment-1 |
| 27 | 7.219047619 | Diagnosis |
| 27 | 6.978461538 | Mid-radiotherapy |
| 27 | 6.95625 | Post-surgery |
| 28 | 27.78 | Diagnosis |
| 28 | 7.1 | Mid-radiotherapy |
| 28 | 8.62 | Post-surgery |
| 29 | 14.86451613 | Diagnosis |
| 29 | 12.16 | Mid-radiotherapy |
| 29 | 8.828571429 | Post-treatment-1 |
| 30 | 10.575 | Diagnosis |
| 30 | 12.75 | Post-surgery |
| 30 | 14.55 | Post-treatment-1 |
| 4 | 14.42033898 | Normal |
| 4 | 8.66 | Normal |
| 4 | 6.92 | Normal |
| 4 | 12.51764706 | Normal |
| 4 | 11.70526316 | Normal |
| 4 | 13.99148936 | Normal |
| 4 | 7.670588235 | Normal |
| 4 | 11.328 | Normal |
| 4 | 8.465454545 | Normal |
| 4 | 8.27 | Normal |
| 4 | 6.498461538 | Normal |
| 4 | 12.72 | Normal |
| 4 | 21.63 | Normal |

After selecting for candidate somatic single nucleotide variants (SNVs) based on plasma profiling and removal of likely germline mutations, we characterized potential false-positives due to clonal hematopoiesis (CH) by comparison with matched PBL profiles. Of the 24 patients with identifiable candidate SNVs, 10 demonstrated identical SNVs within their matched PBL profile with highly correlated mutant allele fractions (MAFs) (R=0.94, p=1.392e⁻⁰⁷, FIG. 2B). With the exception of PIK3CA, genes harboring these SNVs were unique to each patient (FIG. 2C). As genes that are commonly affected by CH, such as DNMT3A, TET2, and ASXL1, were not included within the CAPP-Seq selector, our findings of patient-unique SNVs within matched cfDNA and PBL samples further emphasizes the benefit of this approach over gene level filtering. Plasma samples from 4 patients were strictly positive for SNVs derived from CH (FIG. 2D), suggesting that matched PBL profiling may greatly minimize false-positive detection of ctDNA at low abundance.

After removing candidate SNVs potentially reflective of CH, ctDNA was detected within plasma of 20 patients (median [range]: 3 [1-10] SNVs per patient). To evaluate the plausibility of these SNVs, we compared our results to whole-exome sequencing data from 279 HNSCC tumors published by The Cancer Genome Atlas (TCGA)⁴⁵, observing similarities in frequently mutated genes including TP53 (65% vs. 72%), PIK3CA (20% vs. 21%), FAT1 (15% vs. 23%), and NOTCH1 (10% vs. 19%) (FIG. 2E). Interestingly, two patients presented with single SNVs not found within these genes (GRIN3A and MYC, FIG. 11), demonstrating the added utility of profiling genes with unknown/non-driver effects to increase detection sensitivity OF ctDNA.

Calculating ctDNA abundance based on the mean MAF of SNVs, ctDNA levels ranged from 0.14% to 4.83% (FIG. 2F). This lower limit of detection is similar to that previously described by others utilizing tumor-naïve CAPP-Seq analysis, estimated at ˜0.14%. Including patients with undetectable ctDNA, the median ctDNA abundance across our HNSCC cohort was 0.49%-similar to what has been observed in localized NSCLC by CAPP-Seq.

Tumor-Naive Detection of Methylation-Based ctDNA from Baseline Plasma

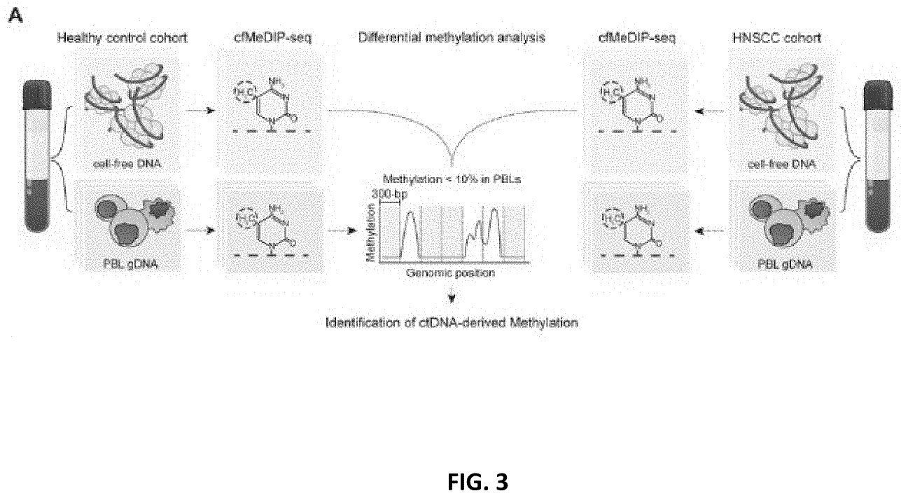

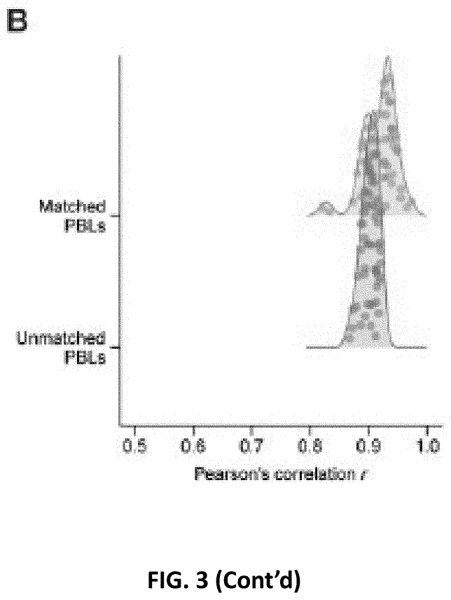

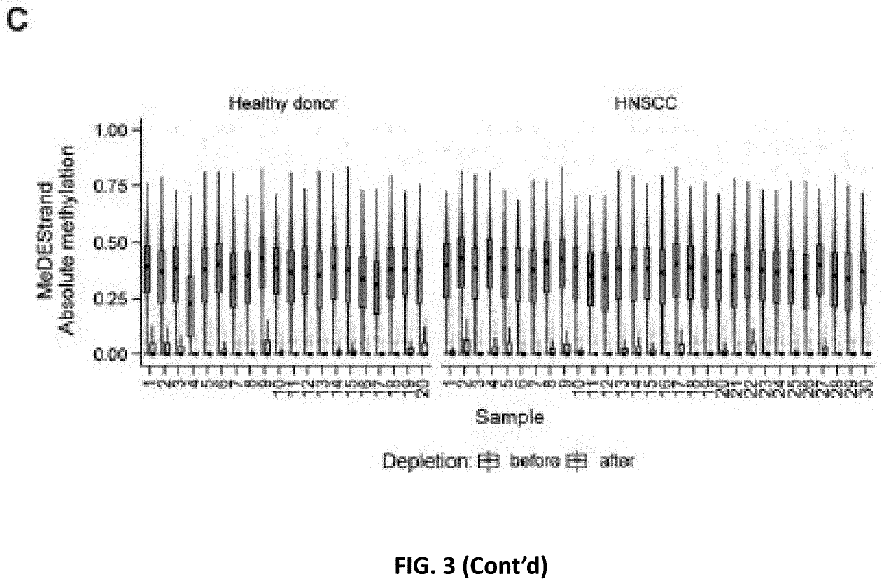

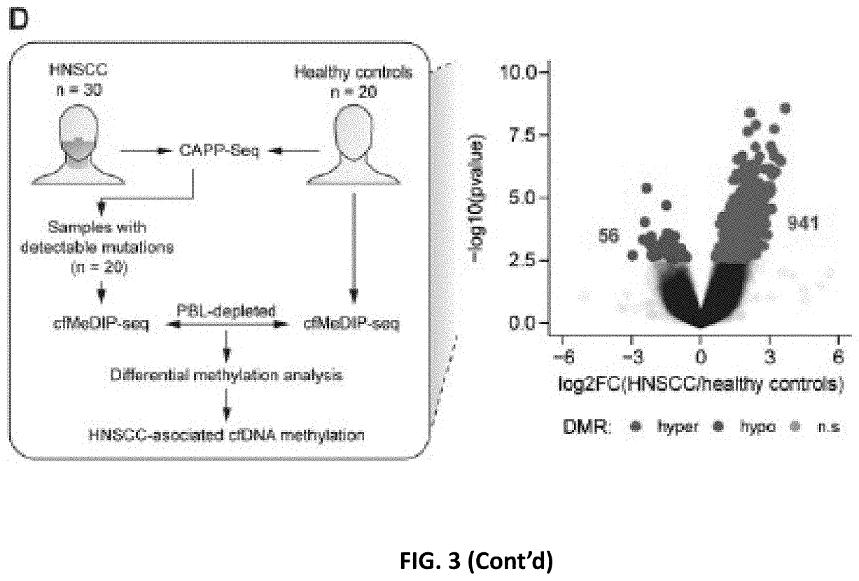

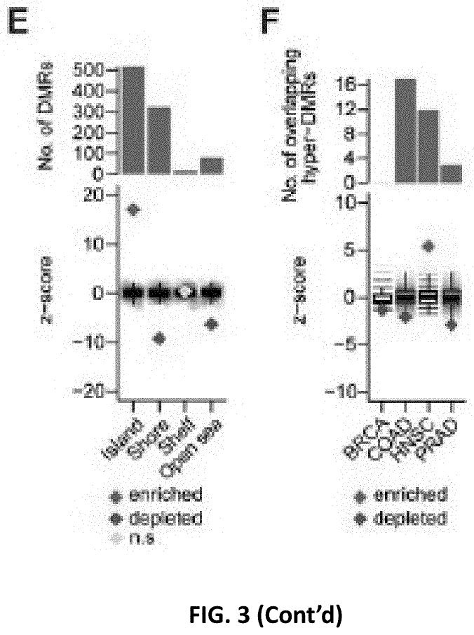

Next, we sought to define ctDNA-associated methylation patterns in the HNSCC and healthy control samples. As the CAPP-Seq results illustrated the impact of false positive mutations arising from PBLs, we reasoned that a reduction of false positive ctDNA-associated methylation may be achieved by removal of PBL-derived DNA methylation signals. Therefore, we used matched PBL MeDIP-seq profiles from the HNSCC and healthy control samples to suppress their contribution to the cell-free DNA methylation signal (FIG. 3A) we evaluated whether matched PBL analysis may also enable methylation-based ctDNA detection (FIG. 3A). Pre-treatment HNSCC and healthy donor plasma as well as PBLs were profiled by cfMeDIP-seq, utilizing 5-10 ng of input DNA. As previously described, methylation abundance was defined within nonoverlapping 300 bp windows across chromosomes 1-22 (n=9,603,454 windows) with read counts normalized to reads per kilobase per million (RPKM) (Methods).

As the anti-5mC antibody utilized for methylation pulldown preferentially binds to DNA fragments at increasing CpG densities, including CpG islands, we first characterized this interaction to identify regions likely to be highly represented within cfMeDIP-seq data. We also applied MeDIP-seq to the HNSCC cell-line FaDu to assess the preferential binding of cancer-derived methylated DNA fragments. Comparing DNA fragment pulldown abundance (median RPKM) across windows with varying numbers of CpGs, we observed increasing enrichment up to ≥8 CpGs for both PBLs and FaDu (FIGS. 12A and 12B). FaDu demonstrated greater enrichment compared to PBLs at ≥8 CpGs per 300 bp window. This result is consistent with the established phenomenon of CpG island hypermethylation in cancer cells including FaDu. Based on these observations, we determined that windows with ≥8 CpGs (n=702,488) may be most informative for ctDNA detection and were therefore utilized for all subsequent analysis.

For patients with localized cancer, the vast majority of plasma cell-free DNA originates from PBLs. Therefore, we sought to exploit PBL MeDIP-seq profiles to bioinformatically suppress this contribution to the cell-free DNA signal. We compared RPKM values for each window within cfMeDIP-seq profiles generated from HNSCC and healthy donor cfDNA, to MeDIP-seq profiles generated from FaDu (1-by-1 comparison), unpaired PBLs (1-by-51 comparison), or paired PBLs (1-by-1 comparison). In accordance with PBLs being the main contributor of plasma cell-free DNA, genome-wide methylation profiles were highly correlated between plasma cell-free DNA and either paired or unpaired PBLs (modal R=0.92 and R=0.91, respectively). The strengths of these correlations likely reflect the known outsize contribution of PBLs to plasma cfDNA. In contrast, correlations were weaker between plasma cell-free DNA and FaDu (modal R=0.78) (FIG. 3B).

To select a threshold of decreased methylation across PBLs while considering preferential pulldown, we scaled and normalized PBL cfMeDIP-seq profiles to absolute methylation levels (0-1) based on logistic regression modelling via the MeDEStrand R package (Methods). We selected 99,997 windows that demonstrated median absolute methylation values <0.1 across healthy donor PBLs. When these windows were applied to left-out HNSCC PBLs we observed similar distributions of absolute methylation to that of the utilized healthy donor PBLs (FIG. 3B), demonstrating generalizability of this approach. Likewise, none of these windows individually showed significantly higher methylation across HNSCC PBLs compared to healthy donor PBLs (FIG. 3C and FIG. 12B), limiting any source of HNSCC-specific PBL methylation that may confound ctDNA detection. In other words, these results confirm that the main source of cfDNA methylation in both control and locoregionally confined HPV-negative HNSCC plasma are derived from PBLs and that bioinformatic removal of PBL-derived methylation may limit signals that confound ctDNA quantification.

Tumor-Naïve Detection of Pre-Treatment Methylation-Based ctDNA

To identify common ctDNA-derived hypermethylated regions within our HNSCC cohort, we performed differential methylation analysis comparing HNSCC patients with detectable ctDNA by CAPP-Seq (n=20) to healthy donors. Utilizing the 99,994 300-bp windows depleted for methylation in PBLs, we identified ctDNA-derived differentially methylated regions (DMRs) by comparing the 20 HNSCC patients with CAPP-Seq-detectable ctDNA to the 20 healthy controls. In total we identified 997 differentially methylated regions (DMRs) (hypermethylated: 941, hypomethylated: 56) across HNSCC samples (FIG. 3C). Approximately half of hypermethylated regions (hyper-DMRs) were found to be immediately adjacent to one another, with blocks of hypermethylation extending up to 1800 base-pairs in length (FIG. 13A). These data suggest the presence of CpG islands within the identified hyper-DMRs. Conversely, no adjacent hypomethylated regions (hypo-DMRs) were observed. Of the 300-bp hyper-DMRs, 47.5% resided in contiguous blocks of hypermethylation signals extending up to 1800 bp in length (FIG. 13A), indicative of CpG islands that typically span 300-3000-bp in length. Indeed, CpG islands were significantly enriched for hyper-DMRs (FIG. 3E). In contrast, CpG islands were significantly depleted for hypo-DMRs (FIG. 13B).

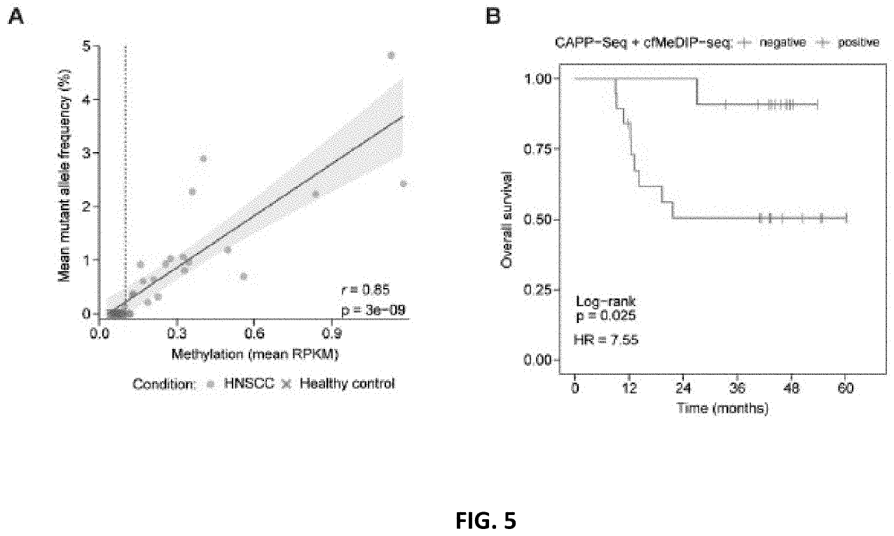

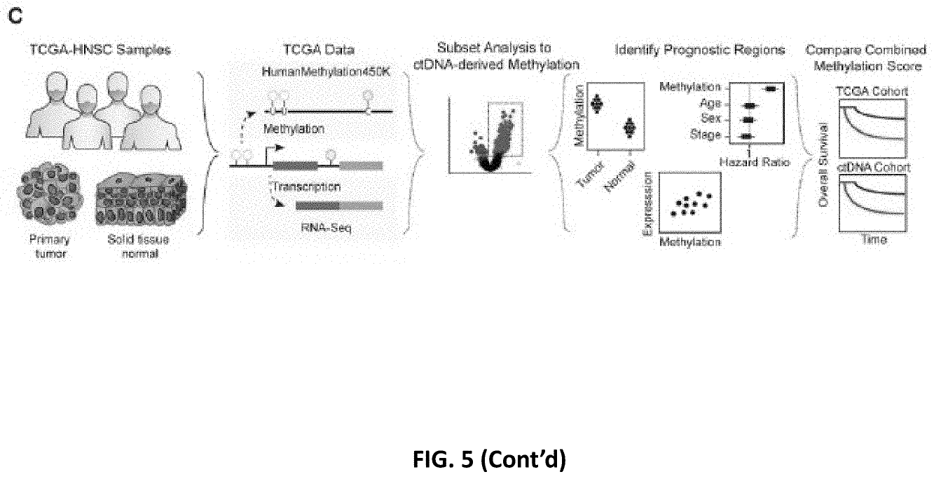

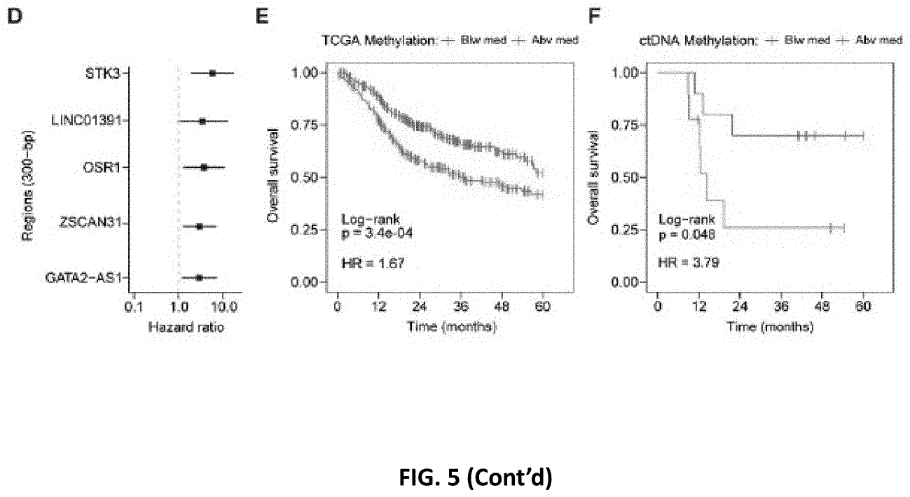

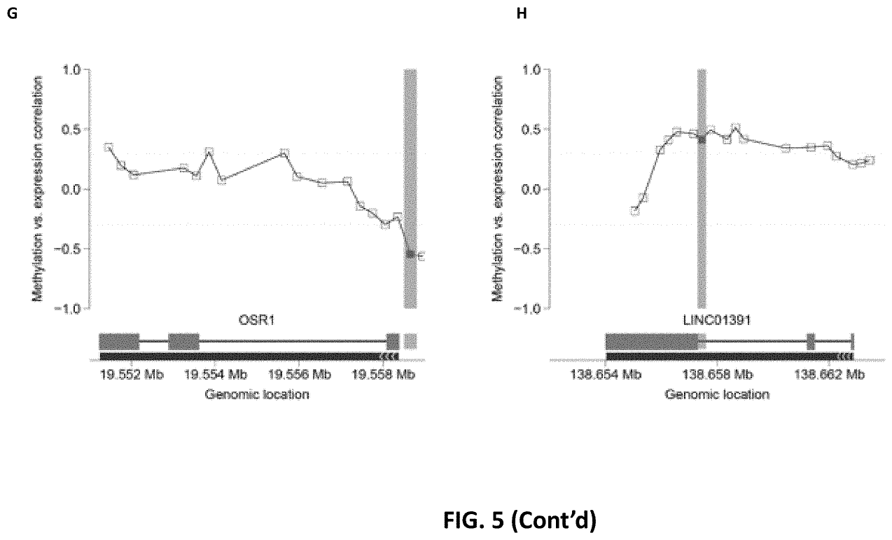

To determine whether these hyper-DMRs were indeed enriched for CpG islands, we next assessed the enrichment of hyper-DMRs for CpG islands, shores, shelves, and open seas by permutation analysis (Methods). As expected, a significant enrichment of CpG islands as well as a significant depletion of shores and open sea was observed within the hyper-DMRs (FIG. 3E). In contrast, the hypo-DMRs were significantly enriched for open sea and depleted for CpG islands (Supplementary FIG. 5B), in accordance with hypomethylation of CpG-sparse regions frequently observed across cancers.

Finally, as methylation of certain regions may distinguish tissue-of-origin as previously described using cfMeDIP-seq, we also investigated whether the hyper-DMRs contained regions specific to HNSCC or other cancers. To identify tumor-specific methylated regions, we utilized HumanMethylation450K (hm450k) data generated from primary tumors provided by TCGA (Methods). Comparing primary tumors from breast invasive carcinoma (BRCA), colon adenocarcinoma (COAD), lung squamous cell carcinoma (LUSC), prostate adenocarcinoma (PRAD), HNSCC, pancreatic adenocarcinoma (PAAD), and PBLs, we identified sufficient hypermethylated CpGs (≥50) specific for BRCA, COAD, PRAD, and HNSCC (Methods) (FIG. 14). As expected, we observed significant enrichment of the plasma-derived DMRs overlapping with HNSC-specific hypermethylated CpGs, as well as a significant depletion of overlap across BRCA-, COAD-, and PRAD-specific hypermethylated CpGs (FIG. 3F), suggesting that the hyper-DMRs contain regions specific to HNSCC origin when compared to various other cancer types.

Mutation-Based and Methylation-Based ctDNA Detection are Highly Concordant

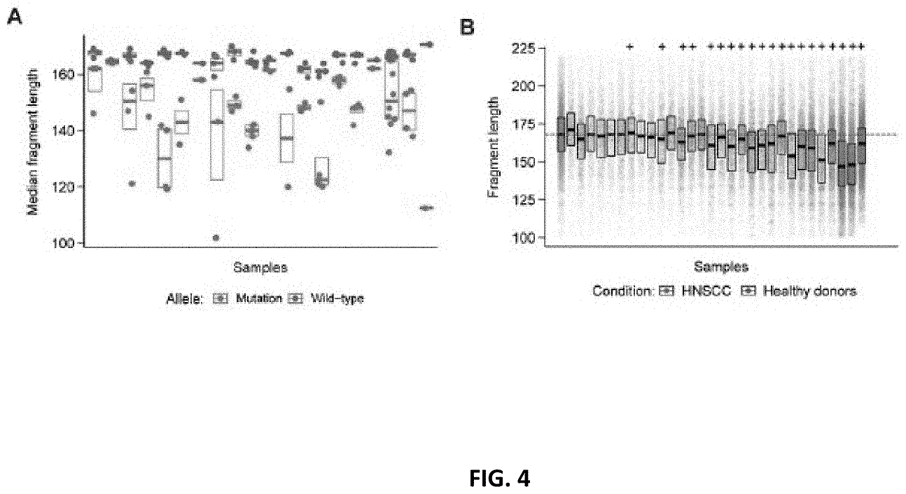

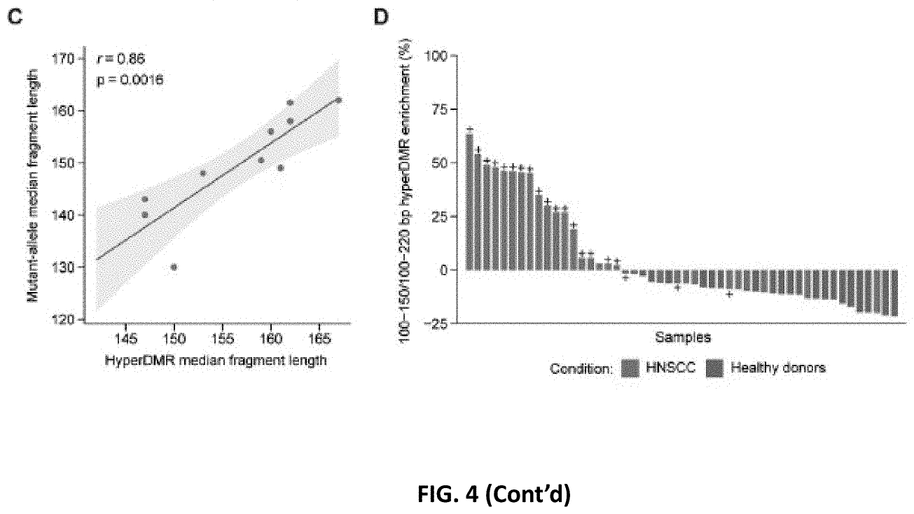

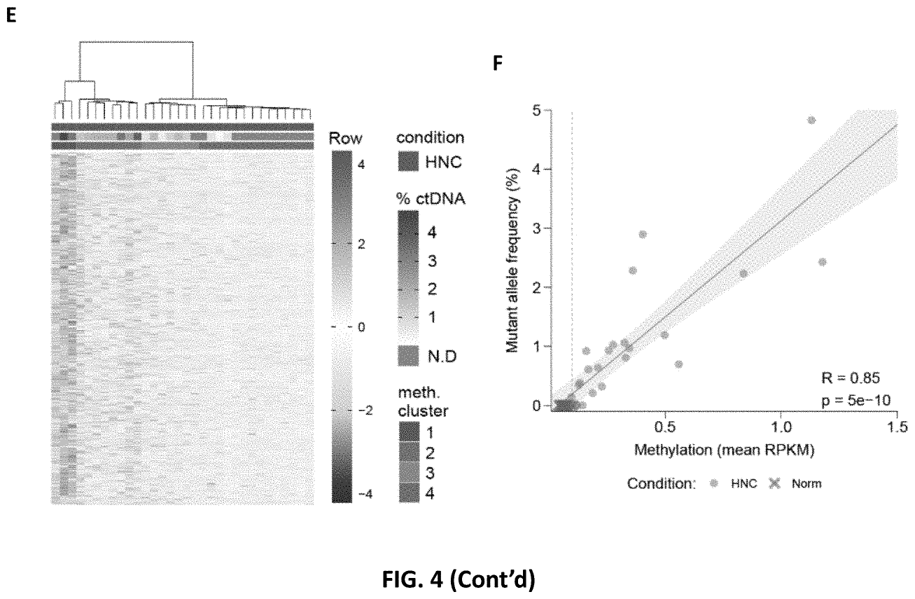

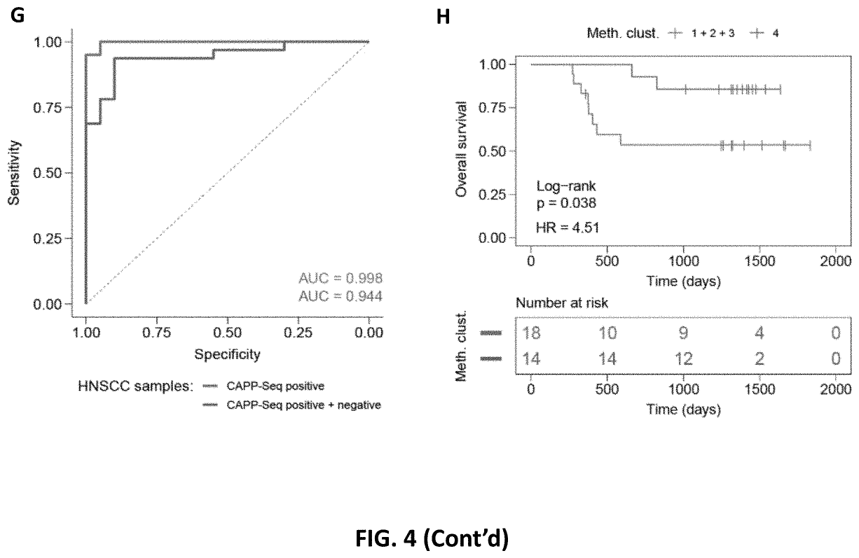

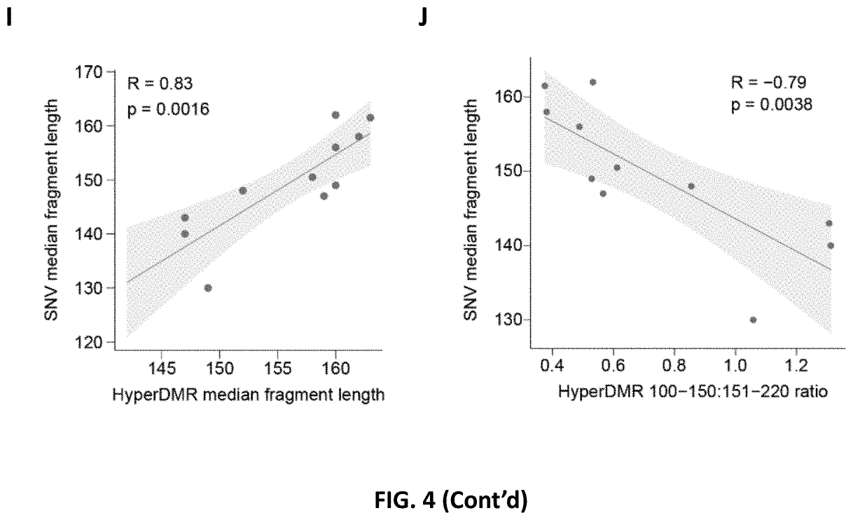

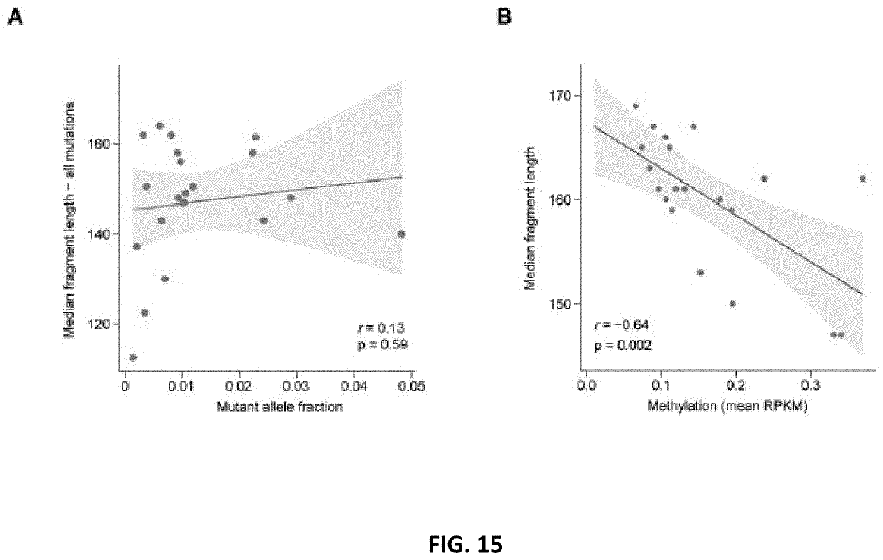

A growing number of studies have described ctDNA to be associated with decreased fragment length compared to healthy sources of plasma cell-free DNA, providing an additional metric for robust tumor-naïve detection. As targeted sequencing has been previously shown to detect ctDNA at reduced fragment length, we first utilized our CAPP-Seq profiles to determine whether we may observe similar trends within HNSCC patients. For each identified SNV per patient (FIG. 2E), we measured the median length of fragments containing the SNV allele as well as the overlapping reference allele. For cases where multiple SNVs were identified within a patient sample, the median value across all SNVs and their reference alleles was used. In accordance with previous findings, we observed a consistent decrease in ctDNA fragment size compared to healthy cell-free DNA across patients (median [range] Δ=−17.5 [1-58] bp) (FIG. 4A). There was no significant association between the mean MAF of these mutations and fragment length (FIG. 15A).

Unlike bisulfite-based DNA methylation approaches, cfMeDIP-seq does not cause DNA degradation and, therefore, preserves the original fragment size distribution. This provides a novel opportunity to map DNA methylation and fragment lengths concomitantly. The distribution of fragment lengths within the previously identified plasma derived hyper-DMRs for each patient was assessed. Due to the nature of these regions having low methylation across our healthy donors, DNA fragments across donors were combined for comparison. Similar to the mutation-based analysis, we observed a reduction in fragment length from 19/20 CAPP-Seq positive patients compared to grouped healthy controls (median [range] Δ=−7 [1-21] bp) (FIG. 4B). This represented a smaller reduction in fragment lengths compared with the mutation-based analysis, possibly due to partial contribution by healthy tissues of cell-free DNA fragments within the hyper-DMRs. Supporting this notion, the samples with the shortest hyper-DMR fragments displayed higher methylated ctDNA abundance (Pearson r=−0.64, p=0.002) (FIG. 15B). When the ratio of small (100-150 bp) versus large (151-220 bp) fragments were used for our hyper-DMRs, an approach previously described to enrich for ctDNA, we observed a similar trend of ctDNA enrichment across the majority of CAPP-Seq positive HNSCC samples (median [range]=28 [−8 to 63]%) (FIG. 4C).

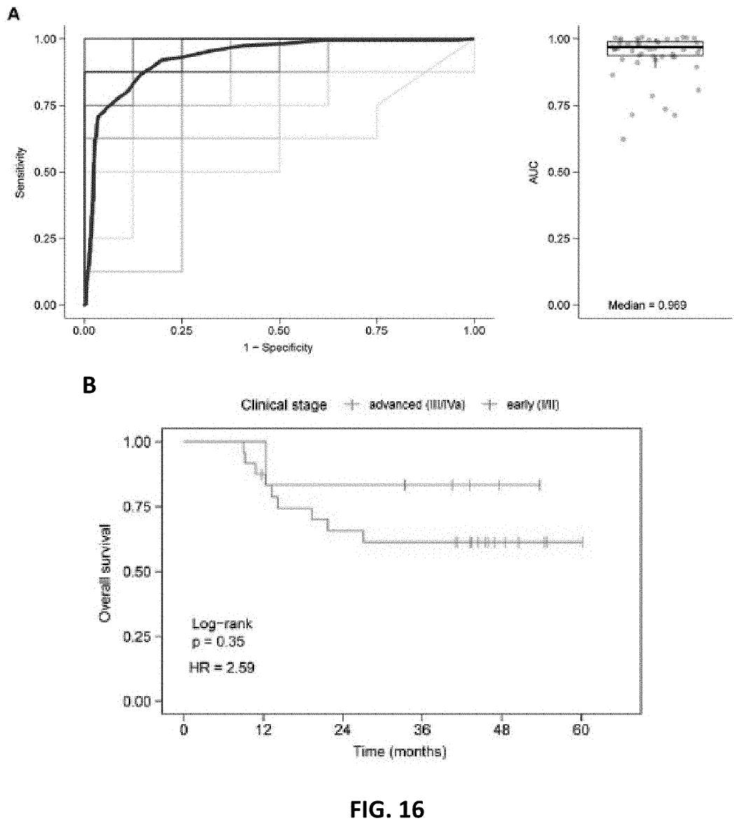

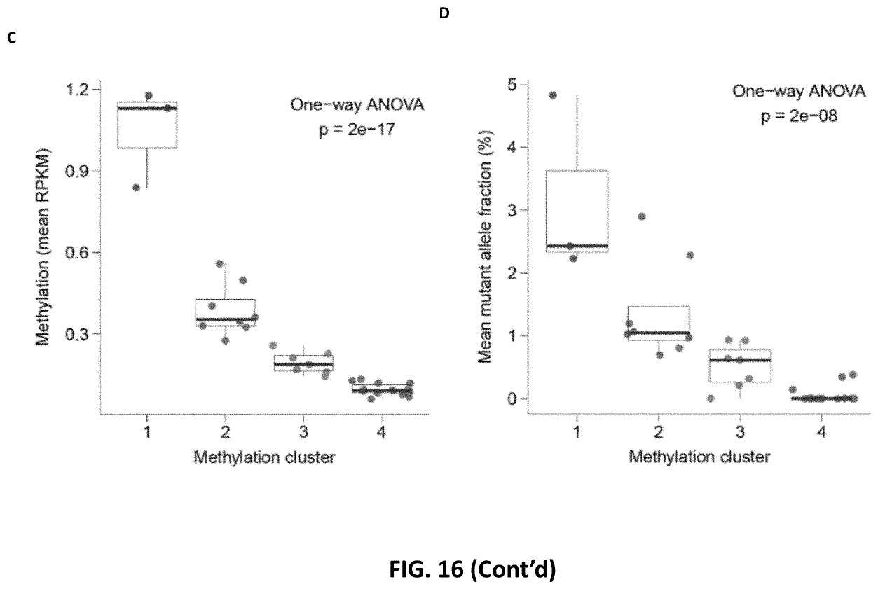

To assess how the plasma cell-free DNA hyper-DMRs identified in our HNSCC cohort may vary across individuals within these small fragments (100-150 bp), we first performed hierarchical clustering. Four dominant clusters emerged utilizing the ConsensusClusterPlus R package, each with distinct levels of methylation across the hyper-DMRs (FIG. 4E and FIG. 16C). Likewise, the three clusters were defined by distinct ctDNA abundance as determined by CAPP-Seq (FIG. 16D), suggesting a potential relationship between mean hyper-DMR methylation and mutation-based ctDNA abundance.

We next investigated whether fragment lengths were concordant between ctDNA molecules Identified by both CAPP-Seq and cfMeDIP-seq, potentially providing an additional layer of validation towards our multimodal approach. To minimize the possibility of background DNA fragments confounding the calculated fragment length of ctDNA within cfMeDIP-seq profiles, we limited analysis to patients above the median methylation levels across hyper-DMRs (n=10 HNSCC patients). Strikingly, ctDNA fragment length was highly concordant between paired CAPP-Seq and cfMeDIP-seq profiles for each patient (Pearson r=0.86, p=0.0016) (FIG. 4C) despite entirely different genomic regions being represented with these two profiling approaches (CAPP-Seq: 43 distinct mutations, cfMeDIP-seq: 941 hyper-DMRs).

To further characterize the relationship between hyper-DMR methylation levels and mutation-based ctDNA abundance, we compared the mean RPKM values across the 941 hyper-DMRs to the mean MAF values determined by CAPP-Seq for each patient. Similar to the trends we observed between methylation clusters, we observed a significant positive correlation (Pearson correlation, R=0.85, p=5e-10) (FIG. 4F). To evaluate the sensitivity of ctDNA detection within these hyper-DMRs by cfMeDIP-seq, we compared mean RPKM values between our HNSCC cohort and healthy donors. For CAPP-Seq positive patients (n=20), ctDNA detection was highly concordant (AUC=0.998) with a marginal decrease in performance upon incorporation of CAPP-Seq negative patients (n=12) (AUC=0.944) (FIG. 4G). Cross validation (n=50 samplings) across CAPP-Seq positive patients and healthy donors resulted in a median AUC value of 0.984 (FIG. 16A), demonstrating the robustness of the approach disclosed herein.

Based on these observations, we evaluated whether we may enrich ctDNA within cfMeDIP-seq profiles by limiting analysis to cell-free DNA fragments of reduced length. We assessed the proportion of cell-free DNA fragments within hyper-DMRs consisting of small (100 to 150 bp) fragments, as similar methods have been described to enrich for ctDNA using non-methylation-based approaches. Indeed, this resulted in ctDNA enrichment across the majority of CAPP-Seq positive HNSCC samples (median [range]=28 [−8 to 63] %) but not for any of the healthy controls (FIG. 4D). Thus, in silico size selection of cell-free DNA fragments enriches for ctDNA within cfMeDIP-seq libraries and may contribute to tumor-naive multimodal ctDNA analysis.

In patients with localized non-metastatic cancer, detection of ctDNA by CAPP-Seq at diagnosis has previously been described to be associated with poor prognosis. Likewise, ctDNA levels as assessed by methylation of SHOX2 and SEPT9 are associated with poor prognosis in HNSCC. Therefore, we asked whether detection or quantification of ctDNA by CAPP-Seq and cfMeDIP-seq at diagnosis would be associated with clinical outcomes within our HNSCC cohort. Indeed, detection of ctDNA by CAPP-Seq (i.e. CAPP-Seq positive vs. CAPP-Seq negative) (hazard ratio [HR]=7.6, log-rank p=0.026; Supplementary FIG. 8D) as well as increased methylation within our previously identified hyper-DMRs (i.e., methylation cluster 1+2+3 vs. methylation cluster 4) (HR=4.51, p=0.038; FIG. 4G), was correlated with shorter survival times. Consistent with this finding, mean RPKM across the hyper-DMRs correlated with cancer stage (Supplementary FIG. 8E).

We next compared the median fragment length of ctDNA identified by either mutation- or methylation-based profiling. To minimize the possibility of background DNA fragments confounding the calculated fragment length of ctDNA within cfMeDIP-seq profiles, we selected patients with high ctDNA abundance as defined by hierarchical clustering (i.e. methylation clusters 1 and 2, FIG. 4D, Supplemental FIG. 8A-B). With this approach, ctDNA fragment length was highly concordant between paired CAPP-Seq and cfMeDIP-seq profiles for each patient (R=0.83, p=0.0016) (FIG. 4H) despite entirely different genomic regions being represented with these two profiling approaches. In addition, similar to our analysis with fragments of all lengths, we observed the same relationship between small fragment ratio and ctDNA fragment length by CAPP-Seq (R=−0.79, p=0.0038) (FIG. 4I).

These results suggest that the similar decrease in fragment length observed from ctDNA detected by CAPP-Seq and cfMeDIP-seq may be a result of inherent properties of the tumor, rather than by genomic region, and that utilization of shorter fragment lengths may contribute to more specific identification of ctDNA.

Application of Multimodal ctDNA Detection for Prognostication

To evaluate the potential clinical applications of tumor-naive multimodal ctDNA analysis, we compared ctDNA with clinical outcomes in the HNSCC cohort. Fragment-length informed cfMeDIP-seq profiles were strongly associated with MAFs in matched CAPP-Seq profiles (Pearson r=0.85, p=3×10-9), suggesting that methylation intensity within the 941 hyper-DMRs is indeed reflective of ctDNA abundance (FIG. 5C). Importantly, cross-validation analysis confirmed the robustness of these hyper-DMRs for detecting ctDNA (FIG. 16C). Patients with ctDNA detected in baseline plasma by both mutation- and methylation-based methods (n=19) were significantly more likely to have advanced disease (i.e., stage III-IVA) (n=18/19) when compared to patients with no detectable ctDNA (n=8/13) (Fisher's exact test p=0.028) and displayed dramatically worse overall survival (hazard ratio [HR]=7.55, 95% confidence interval [CI]=[0.95 to 59.94], log-rank p=0.025) (FIG. 5G). In comparison, stage alone was unable to predict patients with worse overall survival (HR=2.59, 95% CI=[0.32 to 20.46], log-rank p=0.35) (FIG. 16D), further demonstrating the potential clinical utility of multimodal ctDNA profiling.

Due to the known effects of DNA methylation on gene expression and resultant functional activity of cancer drivers, we reasoned that ctDNA methylation patterns at particular loci might have prognostic significance independent of ctDNA abundance. To evaluate whether our previously identified hyper-DMRs contain specific regions associated with prognosis independent of ctDNA abundance, we interrogated DNA methylation, RNA expression, and clinical outcome data provided by the TCGA for all available HNSCC patients (n=520) (FIG. 5C). First, we calculated mean β-values across all CpGs contained within distinct 300-bp windows from TCGA hm450k methylation array data. Limiting analysis to probed hm450k regions overlapping with our plasma-derived hyper-DMRs (n=764/941), we identified 483 hypermethylated regions in primary tumors (n=520) compared to adjacent normal tissue (n=50) (Wilcoxon test, FDR<0.05, log 2FC>1). We observed that several of these hypermethylated regions overlapped or were located near CpGs within genes that are profiled by commercially available methylation-based ctDNA diagnostic tests, including SEPT9 and SHOX2 which have been previously assessed in HNSCC, as well as TWIST1 and ONECUT2 (FIG. 17A). These results provide further evidence supporting the potential clinical relevance of our plasma derived hyper-DMRs.

To further probe the potential clinical utility of these hypermethylated regions held in common by our HNSCC cohort and TCGA HNSC hm450k profiles, we performed univariate Cox proportional-hazards regression across all TCGA HNSCC patients with available hm450k profiles and disease-specific survival (DSS) outcomes (n=493/520). We identified 33 regions that were significantly associated with DSS (p<0.05). To further select prognostic regions likely to have a functional role in tumorigenesis, we compared the methylation levels of each region (n=33) to the expression of surrounding gene transcripts within 2 kb. Next, we used the TCGA HNSCC cohort to identify a subset of the 483 DMRs that were associated with (1) prognosis in multivariable Cox regression and (2) expression of neighboring gene transcripts. Five regions were identified to satisfy both criteria, with increased methylation of each region resulting in higher expression of ZNF323/ZSCAN31, LINC01391, and GATA2-AS1 (FIG. 5G, FIG. 17A-17C, as well as lower expression of STK3/MST2 and OSR1, respectively (FIG. 5H) (FIG. 5D). The regions associated with decreased and increased expression as a result of methylation were found to reside within the promoter or 1^{st} exon/intron and gene body, respectively. We constructed a composite methylation score (CMS) from these 5 regions (Table 6) and stratified the TCGA HNSCC cohort according to this score (FIG. 5E). A higher CMS was significantly associated with inferior survival outcomes (HR=1.67, 95% CI=[1.25, 2.21], log-rank p=3.4×10⁻⁴).

Finally, we evaluated whether the CMS may also provide similar prognostic information when applied to ctDNA. To enrich for ctDNA, analysis of cfMeDIP-seq libraries were limited to fragments between 100-150 bp in length as described above (FIG. 4E). To account for the relative contribution of ctDNA methylation levels provided by the 5 putative prognostic markers, we normalized the cfMeDIP-seq RPKM values from these regions to the entire 941 hyper-DMRs. This produced a similar trend with higher CMS being marginally associated with worse survival (log-rank p=0.1; HR=3.06) (FIG. 5F) suggesting that increased methylation of these putative prognostic regions identified from TCGA may also be informative within cfMeDIP-seq profiles. Moreover, these results highlight how plasma cell-free DNA methylome profiling may be leveraged in combination with existing multi-omic cancer databases for biomarker discovery.

Disease Surveillance after Definitive Treatment by cfMeDIP-Seq

As cfMeDIP-seq achieved sensitive and quantitative ctDNA detection in HNSCC patients, we reasoned that as with CAPP-seq, cfMeDIP-seq may also be capable of monitoring therapy-related changes in ctDNA abundance. To quantify percent ctDNA within posttreatment cfMeDIP-seq profiles, we applied a linear transformation of mean RPKM across the previously identified plasma-derived hyper-DMRs (n=941), limiting fragment size between 100 to 150 bp to further enrich ctDNA. We calculated the detection threshold of 0.2% ctDNA based on the maximum of mean RPKM values observed across all healthy controls. For CAPP-Seq positive HNSCC patients with one or more available post-treatment samples (n=20), cfMeDIP-seq was performed utilizing 10 ng of input cfDNA.

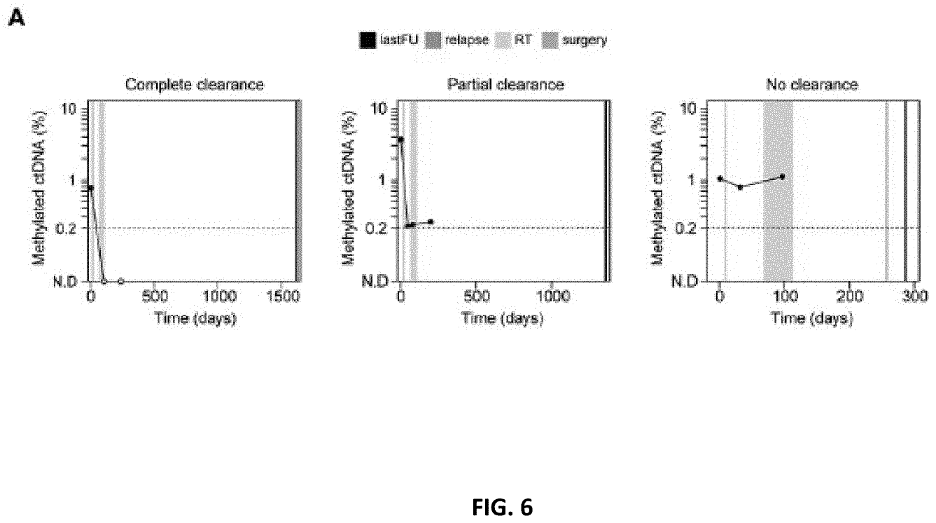

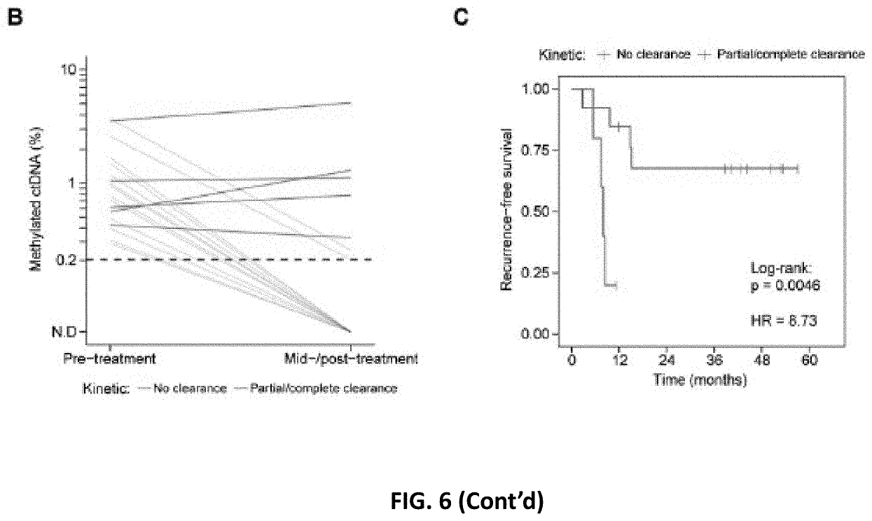

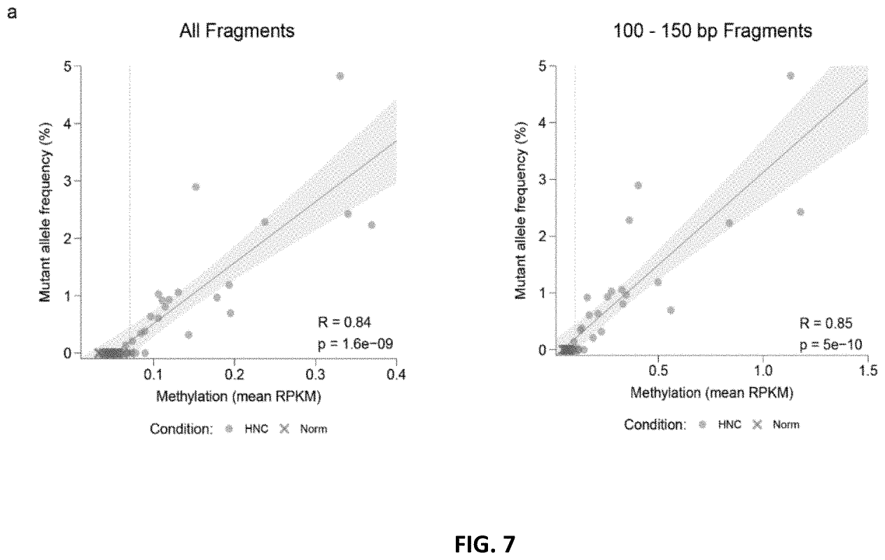

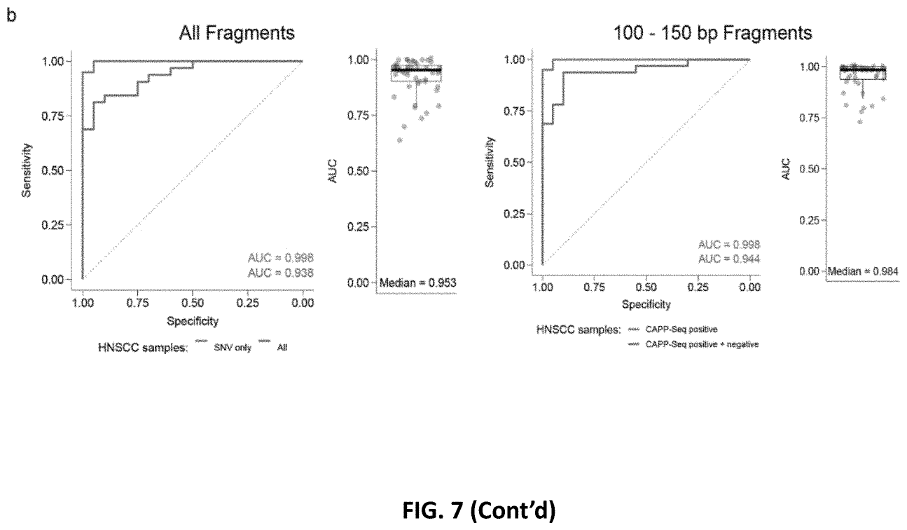

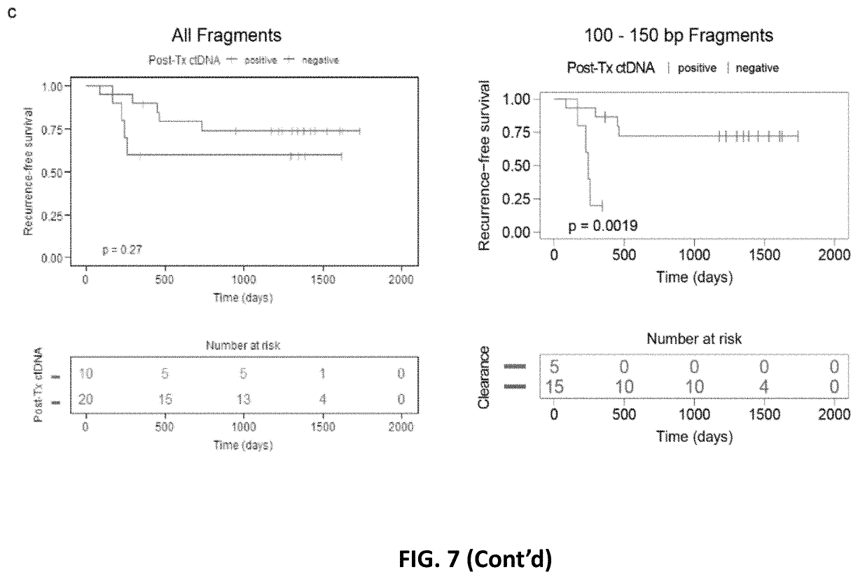

Measuring changes in ctDNA abundance throughout treatment, we observed a variety of kinetics indicative of complete clearance (CC), partial clearance (PC; greater than 90% reduction), or no clearance (NC) (FIG. 6A, Supplementary FIG. 10). Among 18 eligible patients, 5 (28%) demonstrated No Clearance (FIG. 6B). No Clearance patients were more likely to experience disease recurrence compared with those with Complete or Partial Clearance (HR=8.73, 95% CI=[1.5, 50.92], log-rank p=0.0046) (FIG. 6C). Interestingly, all patients with ctDNA abundance greater at last sample collection compared to at diagnosis, demonstrated disease recurrence. In addition, the only patient who did not have documented disease recurrence within this group was lost to follow-up but died within a year after treatment from unknown cause. For the 13 patients with undetectable post-treatment ctDNA by cfMeDIP-seq, 9 remained disease-free with a median of 44.4 months of follow up (min=12.2, max=58.7). Among the other 4 patients, one had persistent disease within regional lymph nodes, and the others experienced relapse 3.5 to 7.7 months (median 7.4 months) after last collection. Of note, these relapses among the patients with undetectable post-treatment ctDNA were considerably more delayed compared to the 4 relapses among the patients with detectable post-treatment ctDNA (median [range]: 3.0 [1.7 to 5.2] months) after last collection. Taken together, these results demonstrate that plasma cell-free DNA methylome profiling by cfMeDIP-seq may be used to assess response to definitive treatment and identify patients at high risk of rapid recurrence.

### Discussion

Broad implementation of ctDNA in clinical settings may be accelerated by methods that can be applied across patients and in the absence of tumor material. In the work described, we evaluated the capabilities of multimodal genome-wide cell-free DNA profiling techniques for tumor-naïve detection of ctDNA within an exploratory cohort of low-ctDNA HNSCC patients. We show that incorporation of matched PBLs improves ctDNA detection using both mutations (i.e., CAPP-Seq) as well as DNA methylation (i.e., cfMeDIP-seq). Furthermore, by utilizing CAPP-Seq to stratify patients with detectable and non-detectable ctDNA, we achieved robust identification of ctDNA-derived methylation patterns. We showed for the first time that biophysical properties of plasma cell-free DNA reflective of tumor origin (i.e., reduced fragment length) are conserved across molecular aberrations and detection platforms. Tumor-naïve ctDNA detection and quantification find multiple clinical uses, and the prognostic association of ctDNA abundance and methylation patterns are investigated.

Tumor-naive ctDNA detection currently encounters several limitations due to low ctDNA abundance. Recent studies have profiled paired PBLs and/or healthy control plasma to identify mutations derived from clonal hematopoiesis, a main contributor to false positive detection of ctDNA; however, the incorporation of orthogonal metrics may further improve accuracy and clinical applicability. Here, we evaluated the capabilities of multimodal genome-wide cell-free DNA profiling techniques for tumor-naive ctDNA detection within a cohort of HNSCC patients with low ctDNA abundance. We demonstrated a high degree of concordance between ctDNA metrics (abundance and fragment lengths) detected by mutation-based and methylation-based profiling methods. Moreover, we showed that tumor-naive multimodal ctDNA profiling may provide value by identifying putative prognostic biomarkers independent of ctDNA abundance, as well as by monitoring ctDNA abundance in serial samples.

Tumor-naïve detection of ctDNA has numerous practical advantages in both research and clinical settings. Recent studies have utilized matched tumor profiling for validation of identified ctDNA-derived regions at low abundance in early stage disease to improve sensitivity. However, one limitation of these approaches is the number of informative regions lost due to sampling heterogeneity of the tumor, which may be further exacerbated when applied to post-treatment ctDNA derived from previously unsampled sub-clones. Additionally, the clinical benefit of these tumor-informed detection methods is limited to cancers readily accessible by biopsy, circumventing one of the main strengths of non-invasive liquid biopsies. By utilizing a tumor-naïve multimodal profiling strategy, we achieved similar results in early stage cancers without the disadvantages of tumor-informed methods.

This is the first work to utilize mutation and methylation profiling for comprehensive detection of ctDNA from a cohort of localized cancer patients. Extending this multimodal profiling approach to other cancer types and disease settings will be important to the continued development of liquid biopsies. Additionally, while numerous ctDNA studies in HNSCC have been described utilizing detection methods based on mutation, methylation, or HPV profiling, here we described the first application of genome-wide mutation/methylation profiling methods identifying previously known targets (i.e. TP53 mutations or SEPT9/SHOX2 methylation) in addition to less-/non-investigated targets.

Tumor-naive detection of ctDNA has numerous practical advantages in both research and clinical settings. Although tumor mutational profiling may identify patient-specific markers for ctDNA detection at low abundance, such personalized approaches rely on high purity tumor samples from cancer types with sufficient mutational load. Mutational profiling for personalized assay design may be costly and time consuming, and it rarely accounts for genomic heterogeneity within primary tumors or across metastatic clones. Additionally, ctDNA detection methods that depend on access to tumor tissue diminish a key advantage of non-invasive liquid biopsies. By integrating independent cell-free DNA properties, we achieved sensitive ctDNA detection in early stage cancers without the disadvantages of tumor-informed methods.

In our analysis, we selected patients with detectable ctDNA by CAPP-Seq in order to identify ctDNA-derived methylation patterns using cfMeDIP-seq. This approach provided additional validation of the tumor-derived nature of plasma cell-free DNA in our cohort. The ctDNA methylation patterns were able to quantify ctDNA abundance in a similar manner to ctDNA mutations. In addition, methylation patterns revealed the tumor-of-origin and identified putative prognostic and dynamic biomarkers. The combination of CAPP-Seq and cfMeDIP-seq enabled an in-depth molecular characterization of low-abundance ctDNA. Mutation-based ctDNA quantification contributed to the discovery of HNSCC-specific hyper-DMRs in plasma, some of which were confirmed to be prognostic even after adjusting for ctDNA abundance. Thus, simultaneous profiling of mutations and methylation may complement one another by revealing quantitative, tissue-specific, and prognostic ctDNA biomarkers. Moreover, methylome profiling may prove particularly useful in cancer types with few recurrent or clonal mutations.

Similar to previous studies, we also observed a decreased in ctDNA fragment length compared to healthy donor cell-free DNA using both mutation- and methylation-based approaches. Unlike healthy cell-free DNA, which is consistently at ˜166-167 bp on average, the length of ctDNA between patients may be highly variable. Factors that influence ctDNA fragment length may include position-dependant fragmentation⁴⁹, metastatic vs. non-metastatic disease⁷³, as well as dysregulated kinetics of various intra/extracellular DNases responsible for healthy cell-free DNA fragmentation⁷⁴. Interestingly, we observed high concordance between fragment lengths of ctDNA identified by CAPP-Seq and cfMeDIP-seq for eligible patients despite both techniques probing different regions and tumor-derived aberrations. These compelling data provide further evidence regarding the relevance and reproducibility of plasma cell-free DNA fragmentation in cancer patients.

We observed that detectable ctDNA by CAPP-Seq or elevated ctDNA abundance by cfMeDIP-seq, was associated with poor prognosis within our HNSCC cohort. These results are in accordance with previous HNSCC ctDNA studies, where detection of ctDNA by methylation⁵⁶, as well as increased abundance by copy number aberrations⁷⁵ or HPV detection⁷⁶, identified high-risk patients. There was an imperfect association with tumor stage, suggesting that other unmeasured features of tumor biology may contribute to ctDNA abundance.

To our knowledge, no study has previously identified prognostic regions in HNSCC cell-free DNA independent of ctDNA detection/abundance, perhaps in part due to limitation of commonly used ctDNA detection methods. We demonstrated that cell-free DNA methylome profiles may serve as a discovery tool, which in conjunction with TCGA data, identified novel prognostic methylation biomarkers in HNSCC. A composite methylation score comprised of 5 DMRs demonstrated consistent prognostic associations across methylation detection platforms (hm450k and cfMeDIP-seq) and biospecimen types (tumor tissue and plasma cell-free DNA). Although future larger cohorts are needed to validate our findings, this study indicates that genome-wide identification of methylated regions by cfMeDIP-seq may enable discovery of novel prognostic biomarkers.

The performance of cfMeDIP-seq was evaluated in connection with disease prognosis. By applying a stringent threshold greater than ˜0.2% ctDNA post-treatment as detectable disease, we were able to predict disease recurrence for 4 out of 9 patients. For the remaining 5 patients that relapsed (n=4) or had persistent disease (n=1), who failed to have detectable ctDNA post-treatment, we observed typically longer times to recurrence suggesting that the fraction of ctDNA at those timepoints may have been below cfMeDIP-seq's lower limit of detection. In subsequent studies utilizing cfMeDIP-seq for tumor-naïve disease surveillance, more frequent plasma collection post-treatment may help address these limitations.

As we have demonstrated the potential clinical utility of multimodal profiling within localized disease and HNSCC, these methods contribute to future biomarker discovery and ultimately clinal utility for patients with a variety of cancer types. This study makes multiple notable contributions. It is the first to combine analyses of cell-free DNA mutations, methylation, and fragment lengths. Moreover, we methodically profiled plasma samples and paired PBLs from both HNSCC patients and risk-matched healthy controls. These analyses have revealed key insights regarding the optimal handling of multimodal profiling for ctDNA detection and characterization. For instance, our unique approaches to removing the contributing methylation signals from leukocytes and using fragment length characteristics to enrich for tumor-derived methylation will prove useful for future studies.

In conclusion, we demonstrate that tumor-naïve CAPP-Seq profiling of ctDNA enables high-confidence identification of ctDNA-derived methylation by cfMeDIP-seq. Utilizing the strength of epigenetic profiling by cfMeDIP-seq, we further show that these ctDNA-derived methylated regions demonstrate potential as markers of tumor-of-origin, prognosis, and treatment response. The incorporation of several approaches that we have described for improved sensitivity of ctDNA detection by cfMeDIP-seq in HNSCC, such as PBL-depleted windows and restriction of analysis to short fragments, may also be applied to various other localized cancers for clinical benefit. The disclosed framework are widely applicable to other clinical settings where tumor tissue availability may be limited.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

1-54. (canceled)

55. A method for processing a cell-free nucleic acid sample of a subject to determine whether said subject has or is at risk of having a disease, comprising:
(a) providing said cell-free nucleic acid sample comprising a plurality of nucleic acid molecules;
(b) subjecting said plurality of nucleic acid molecules or derivatives thereof to sequencing to generate a plurality of sequencing reads;
(c) computer processing said plurality of sequencing reads to identify, for said plurality of nucleic acid molecules, (i) a methylation profile, (ii) a mutation profile, and (iii) a fragment length profile; and
(d) using at least said methylation profile, said mutation profile and said fragment length profile to determine whether said subject has or is at risk of having said disease.

56. The method of claim 55, wherein the disease comprises a cancer.

57. The method of claim 56, wherein the cancer is selected from the group consisting of the cancer is selected from the group consisting of adrenal cancer, anal cancer, bile duct cancer, bladder cancer, bone cancer, brain/cns tumors, breast cancer, castleman disease, cervical cancer, colon/rectum cancer, endometrial cancer, esophagus cancer, ewing family of tumors, eye cancer, gallbladder cancer, gastrointestinal carcinoid tumors, gastrointestinal stromal tumor (gist), gestational trophoblastic disease, hodgkin disease, kaposi sarcoma, kidney cancer, laryngeal and hypopharyngeal cancer, leukemia (acute lymphocytic, acute myeloid, chronic lymphocytic, chronic myeloid, chronic myelomonocytic), liver cancer, lung cancer (non-small cell, small cell, lung carcinoid tumor), lymphoma, lymphoma of the skin, malignant mesothelioma, multiple myeloma, myelodysplastic syndrome, nasal cavity and paranasal sinus cancer, nasopharyngeal cancer, neuroblastoma, non-hodgkin lymphoma, oral cavity and oropharyngeal cancer, osteosarcoma, ovarian cancer, penile cancer, pituitary tumors, prostate cancer, retinoblastoma, rhabdomyosarcoma, salivary gland cancer, sarcoma—adult soft tissue cancer, skin cancer (basal and squamous cell, melanoma, merkel cell), small intestine cancer, stomach cancer, testicular cancer, thymus cancer, thyroid cancer, uterine sarcoma, vaginal cancer, vulvar cancer, waldenstrom macroglobulinemia, wilms tumor, squamous cell carcinoma, and head and neck squamous cell carcinoma.

58. The method of claim 57, wherein the cancer is squamous cell carcinoma.

59. The method of claim 58, wherein the cancer is head and neck squamous cell carcinoma.

60. The method of claim 55, wherein said plurality of cell-free nucleic acid molecules comprises circulating tumor nucleic acid molecules.

61. The method of claim 60, wherein the circulating tumor nucleic acid molecules comprises circulating tumor DNA.

62. The method of claim 60, wherein the circulating tumor nucleic acid molecules comprises circulating tumor RNA.

63. The method of claim 55, wherein said methylation profile comprises a plurality of Differentially Methylated Regions (DMRs).

64. The method of claim 63, wherein said plurality of DMRs is ctDNA derived.

65. The method of claim 63, wherein said plurality of DMRs derived from peripheral blood leukocytes is removed from said methylation profile.

66. The method of claim 63, wherein said plurality of DMRs comprises at least about 56 genomic regions with hypo-methylation levels compared to corresponding genomic regions from a normal healthy subject.

67. The method of claim 63, wherein said plurality of DMRs comprises at least about 941 genomic regions with hyper-methylation levels compared to corresponding genomic regions from a normal healthy subject.

68. The method of claim 63, wherein a DMR of the plurality of DMRs comprises a size of at least about 300 bp.

69. The method of claim 68, wherein said DMR comprises a size of at least about 100 bp to at least about 200 bp.

70. The method of claim 68, wherein said DMR comprises a size of at least about 100 bp to at least about 150 bp.

71. The method of claim 63, wherein a DMR of the plurality of DMRs comprises at least 8 CpG genomic islands.

72. The method of claim 66, wherein said normal healthy subject comprises a same set of risk factors as said subject.

73. The method of claim 55, wherein said mutation profile comprises a missense variant, a nonsense variant, a deletion variant, an insertion variant, a duplication variant, an inversion variant, a frameshift variant, or a repeat expansion variant.

74. The method of claim 55, wherein any variant that is present in a genomic DNA sample obtained from a plurality of peripheral blood leukocytes, wherein said plurality of peripheral blood leukocytes is obtained from said subject, is removed from said mutation profile.

75. The method of claim 55, wherein any variant that is derived from clonal hematopoiesis is removed from said mutation profile.

76. The method of claim 75, wherein said mutation profile does not comprise a variant of gene DNMT3A, TET2, or ASXL1.

77. The method of claim 75, wherein said mutation profile does not comprise a canonical cancer driver gene.

78. The method of claim 75, wherein said mutation profile comprises a non-canonical cancer driver gene, where said non-canonical cancer driver gene is GRIN3A or MYC.

79. The method of claim 55, wherein said fragment length profile comprises selecting cell free nucleic acid molecules based on a range of fragment length of about at least 80 bp to 170 bp.

80. The method of claim 55, wherein said fragment length profile comprises selecting cell free nucleic acid molecules based on a range of fragment length of about at least 100 bp to 150 bp.

81. The method of claim 60, wherein said circulating tumor nucleic acid molecules are enriched.

82. The method of claim 55, further comprising mixing said cell free nucleic acid sample with a filler DNA molecules to yield a DNA mixture.

83. The method of claim 82, wherein said filler DNA molecules comprise a length of about 50 bp to 800 bp.

84. The method of claim 82, wherein said filler DNA molecules comprise a length of about 100 bp to 600 bp.

85. The method of claim 82, wherein said filler DNA molecules comprises at least about 5% methylated filler DNA molecules.

86. The method of claim 82, wherein said filler DNA molecules comprises at least about 20% methylated filler DNA.

87. The method of claim 82, wherein said filler DNA molecules comprises at least about 30% methylated filler DNA.

88. The method of claim 82, wherein said filler DNA molecules comprises at least about 50% methylated filler DNA.

89. The method of claim 55, further comprising incubating said DNA mixture with a binder that is configured to bind methylated nucleotides to generate an enriched sample.

90. The method of claim 89, wherein said binder comprises a protein comprising a methyl-CpG-binding domain.

91. The method of claim 90, wherein said protein is a MBD2 protein.

92. The method of claim 89, wherein said binder comprises an antibody.

93. The method of claim 92, wherein said antibody is a 5-MeC antibody.

94. The method of claim 9, wherein said antibody is a 5-hydroxymethyl cytosine antibody.

95. The method of claim 55, wherein said determining comprises sequencing said plurality of sequencing reads, and wherein said sequencing does not comprise bisulfite sequencing.

96. The method of claim 55, wherein said cell-free nucleic acid sample comprises a blood sample.

97. The method of claim 96, wherein said blood sample comprises a plasma sample.

98. The method of claim 55, further comprising detecting an origin of cancer tissue.

99. The method of claim 55, further comprising generating a report comprising a prognosis of said subject's survival rate.

100. The method of claim 55, further comprising providing a treatment to said subject.

101. The method of claim 100, subsequent to treatment of said disease, further comprising providing a second report indicating whether said treatment is effective.

102. A method for determining whether a subject has or is at risk of having a condition, comprising:
(a) assaying a cell-free nucleic acid molecule from at least a portion of a sample from said subject;
(b) detecting a methylation level of at least a portion of said cell-free nucleic acid molecule comprised in a differentially methylated region (DMR) listed in Table 5; and
(c) comparing, using at least one computer processor, said methylation level detected in (b) to a methylation level of corresponding portion(s) of said cell-free nucleic acid molecules comprised in said DMR listed in Table 5.

103. The method of claim 102, wherein said cell-free nucleic acid molecule comprises ctDNA.

104. The method of claim 102, wherein said assaying comprises performing a sequence analysis, and wherein said sequence analysis comprises cell-free methylated DNA immunoprecipitation (cfMeDIP) sequencing.

105. The method of claim 102, wherein said detecting comprises measuring a methylation level of at least a portion of said nucleic acid molecule comprised in: six or more, ten or more, fifteen or more, twenty or more, thirty or more, forty or more, fifty or more, sixty or more, seventy or more, eighty or more, ninety or more, or one hundred or more DMRs listed in Table 5.

106. A method for determining whether a subject has a higher survival rate after receiving a treatment for a disease, comprising:
(a) assaying a cell-free nucleic acid molecule from at least a portion of a sample from said subject;
(b) detecting a methylation level of at least a portion of said cell-free nucleic acid molecule comprised in a differentially methylated region (DMR) listed in Table 6; and
(c) processing, using at least one computer processor, said methylation level detected in (b) to a methylation level of corresponding portion(s) of said cell-free nucleic acid molecules comprised in said DMR listed in Table 6.

107. The method of claim 106, wherein said cell-free nucleic acid molecule comprises ctDNA.

108. The method of claim 106, wherein said detecting comprises providing a composite methylation score (CMS).

109. The method of claim 107, wherein said CMS comprises a sum of beta-values of DMRs listed in Table 6.

110. The method of claim 107, wherein a higher CMS indicates an inferior survival for said subject.

111. The method of claim 107, wherein said CMS is not dependent on an abundance of ctDNA.

112. The method of claim 106, wherein said disease is squamous cell carcinoma.

113. The method of claim 112, wherein the squamous cell carcinoma is head and neck squamous cell carcinoma.

114. The method of claim 106, further comprising selecting cell free nucleic acid molecules based on a range of fragment length of about at least 80 bp to 170 bp.

115-116. (canceled)
